(19) United States
(12) Patent Application Publication
(10) Pub. No.: US 2009/0191217 A1
(43) Pub. Date: Jul. 30, 2009

(54) Anti-IL-1R1 Single Domain Antibodies And Therapeutic Uses

(76) Inventors: Ruud M. de Wildt, Cambridge (GB); Philip D. Drew, Cambridge (GB); Ian M. Tomlinson, Great Shelford (GB); Mary Fitzgerald, Essex (GB); Craig Fox, Essex (GB); Steve Holmes, Great Chishill (GB)

Correspondence Address: MCDERMOTT WILL & EMERY LLP, 28 STATE STREET, BOSTON, MA, 02109-1775, US

(21) Appl. No.: 11/791,781

(22) PCT Filed: Dec. 1, 2005
(86) PCT No.: PCT/GB2005/004601

§ 371 Date: Aug. 23, 2007

Related U.S. Application Data
(60) Provisional application No. 60/632,361, filed on Dec. 2, 2004.

(30) Foreign Application Priority Data
Oct. 24, 2005 (GB) 0521621.3

(51) Int. Cl. A61K 39/395; A61M 5/178; A61K 9/48; A61K 9/20; A61M 37/00; A61M 25/00; A61M 15/00

(52) U.S. Cl. 424/158.1; 604/187; 424/451; 424/464; 604/289; 604/523; 128/200.23

(57) ABSTRACT

Disclosed is the use of an antagonist of Interleukin 1 receptor type 1 (IL-1R1) for the manufacture of a medicament treating, preventing or suppressing lung inflammation or a respiratory disease. In some embodiments of the described invention, the medicament is for local administration to pulmonary tissue. Also disclosed are methods for treating lung inflammation or a respiratory disease.

### RELATED APPLICATIONS

This application is a continuation-in-part of International Application No. PCT/GB2005/002163, which designated the United States and was filed on May 31, 2005, which claims the benefit of U.S. Provisional Application No. 60/632,361, filed on Dec. 2, 2004; and this application claims priority under 35 U.S.C. § 119 or 365 to United Kingdom, Application No. 0521621.3, filed Oct. 24, 2005. The entire teachings of the above applications are incorporated herein by reference.

### BACKGROUND OF THE INVENTION

The in vivo use of many agents with therapeutic or diagnostic potential is not possible. Larger agents that have in vivo serum half-lives that are sufficiently long to allow for therapeutic or diagnostic efficacy often are unable to penetrate tissues or organs to produces a desired therapeutic or diagnostic effect at a desired location. Smaller agents are able to enter tissues and organs, but frequently have short in vivo serum half-lives, and are rapidly cleared from the systemic circulation. For example, the in vivo serum half-life of dAb monomers is about 30 minutes. (See, Examples 9 and 13 of WO 2004/081026 A2.) Similarly, the in vivo serum half-life of antigen-binding fragments of antibodies, particularly Fv fragments, is also short and makes them unsuitable for many in vivo therapeutic and diagnostic applications. (Peters et al., Science 286(5439):434 (1999).) Further, altering or modifying such agents to increase the in vivo serum half-life can reduce the activity of the agent.

Certain agents that bind Interleukin 1 Receptor Type 1 (IL-1R1) and neutralize its activity have proven to be effective therapeutic agents for certain inflammatory conditions, such as moderately to severely active rheumatoid arthritis. However, other agents that bind IL-1R1, such as the anti-IL-1R1 antibody AMG 108 (Amgen) have failed to meet primary endpoints in clinical studies. No agents that bind and antagonize IL-1R1 have been demonstrated to be effective in treating lung inflammation or respiratory diseases, such as chronic obstructive pulmonary disease (COPD).

A need exists for improved agents that antagonize IL-1R1 and method for administering such agents to treat lung inflammation and lung disease.

### SUMMARY OF THE INVENTION

The invention relates to use of an antagonist of Interleukin-1 Receptor Type 1 (IL-1R1) for the manufacture of a medicament for treating a respiratory disease, and to method of treating a respiratory disease that comprise administering an antagonists of IL-1R1. The respiratory disease can be, for example, selected from the group consisting of lung inflammation, chronic obstructive pulmonary disease, asthma, pneumonia, hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease, influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X, pulmonary hypertension, pulmonary nocardiosis, pulmonary tuberculosis, pulmonary veno-occlusive disease, rheumatoid lung disease, sarcoidosis, and Wegener's granulomatosis.

The medicament can be for systemic or local administration. In some embodiments, the medicament is for intraperoneal or subcutaneous administration. In other embodiments, the medicament is for local administration to pulmonary tissue, for example, the medicament can be for inhalation or intranasal administration.

In some embodiments the medicament further comprises antagonist of Tumor Necrosis Factor Receptor 1 (TNFR1, p55), or is for administration together with an antagonist of Tumor Necrosis Factor Receptor 1 (TNFR1, p55).

In one aspect, the method relates to use of an antagonist of IL-1R1 for manufacture of a medicament for treating a respiratory. In some embodiments, the antagonist of IL-1R1 comprises a polypeptide domain that has binding specificity for Interleukin-1 Receptor Type 1 (IL-1R1) and inhibits binding of a ligand selected from the group consisting of Interleukin-1α (IL-1α) and Interleukin-1β (IL-1β) to IL-1R1. For example, the polypeptide domain that has binding specificity for IL-1R1 can be selected from the group consisting of an antibody or antigen-binding fragment thereof, Interleukin-1 receptor antagonist (IL-1ra) or a functional variant of IL-1ra.

Preferably, the polypeptide domain that has binding specificity for IL-1R1 inhibits binding of said ligand to IL-1R1 with an IC50 that is ≦1 μM. In some embodiments, the polypeptide domain that has binding specificity for IL-1R1 inhibits IL-1α- or IL-1β-induced release of Interleukin-8 by MRC-5 cells (ATCC Accession No. CCL-171) in an in vitro assay with a ND50 that is ≦1 μM, or preferably ≦1 nM. In other embodiments, the polypeptide domain that has binding specificity for IL-1R1 inhibits IL-1α- or IL-1β-induced release of Interleukin-6 in a whole blood assay with a ND50 that is ≦1 μM.

In particular embodiments, the polypeptide domain that has binding specificity for IL-1R1 is an antigen-binding fragment of an antibody, and said antigen-binding fragment is an immunoglobulin single variable domain. Preferably, one or more of the framework regions (FR) in said immunoglobulin single variable domain comprise (a) the amino acid sequence of a human framework region, (b) at least 8 contiguous amino acids of the amino acid sequence of a human framework region, or (c) an amino acid sequence encoded by a human germline antibody gene segment, wherein said framework regions are as defined by Kabat. The amino acid sequences of one or more framework regions in said immunoglobulin single variable domain can be the same as the amino acid sequence of a corresponding framework region encoded by a human germline antibody gene segment, or the amino acid sequences of one or more of said framework regions can collectively comprise up to 5 amino acid differences relative to the corresponding framework regions encoded by a human germline antibody gene segment. In some embodiments, the amino acid sequences of FR1, FR2, FR3 and FR4 in the immunoglobulin single variable domain are the same as the amino acid sequences of corresponding framework regions encoded by a human germline antibody gene segment, or the amino acid sequences of FR1, FR2, FR3 and FR4 collectively contain up to 10 amino acid differences relative to the corresponding framework regions encoded by a human germline antibody gene segment. In particular embodiments, the immunoglobulin single variable domain comprises FR1, FR2 and FR3 regions, and the amino acid sequence of said FR1, FR2 and FR3 are the same as the amino acid sequences of corresponding framework regions encoded by a human germline antibody gene segment. In more particular embodiments, the human germline antibody gene segment comprises is DPK9 and JK1.

The antagonist of IL-1R1 can comprise an immunoglobulin single variable domain that competes for binding to IL-1R1 with a dAb selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO: 129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

In particular embodiments, the antagonist of IL-1R1 comprises an immunoglobulin single variable domain immunoglobulin single variable domain comprises an amino acid sequence that has at least about 90% amino acid sequence identity with an amino acid sequence selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

Preferably, the antagonist of IL-1R1 comprises a polypeptide domain that has binding specificity for IL-1R1 binds human IL-1R1 with an affinity (KD) of about 300 nM to about 5 pM, as determined by surface plasmon resonance.

In some embodiments, the antagonist of IL-1R1 further comprises a half-life extending moiety. The half-life extending moiety can be, for example, a polyalkylene glycol moiety, serum albumin or a fragment thereof, transferrin receptor or a transferrin-binding portion thereof, or an antibody or antibody fragment comprising a binding site for a polypeptide that enhances half-life in vivo. In some embodiments, the half-life extending moiety is a polyethylene glycol moiety.

In other embodiments, the half-life extending moiety is an antibody or antibody fragment comprising a binding site for serum albumin or neonatal Fc receptor. For example, the half-life extending moiety can be an immunoglobulin single variable domain that binds serum albumin or neonatal Fc receptor.

In particular embodiments, the half-life extending moiety is an immunoglobulin single variable domain that competes with a dAb selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7h-8 (SEQ ID NO:746), DOM7r-13 (SEQ ID NO:747), DOM7r-14 (SEQ ID NO:748), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), DOM7r-33 (SEQ ID NO:767), Sequence A (SEQ ID NO:768), Sequence B (SEQ ID NO:769), Sequence C (SEQ ID NO:770), Sequence D (SEQ ID NO:771), Sequence E (SEQ ID NO:772), Sequence F (SEQ ID NO:773), Sequence G (SEQ ID NO:774), Sequence H (SEQ ID NO:775), Sequence I (SEQ ID NO:776), Sequence J (SEQ ID NO:777), Sequence K (SEQ ID NO:778), Sequence L (SEQ ID NO:779), Sequence M (SEQ ID NO:780), Sequence N (SEQ ID NO:781), Sequence O (SEQ ID NO:782), Sequence P (SEQ ID NO:783), and Sequence Q (SEQ ID NO:784), for binding to human serum albumin.

In other embodiments, the half-life extending moiety is an immunoglobulin single variable domain that binds human serum albumin and comprises an amino acid sequence selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7h-8 (SEQ ID NO:746), DOM7r-13 (SEQ ID NO:747), DOM7r-14 (SEQ ID NO:748), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), DOM7r-33 (SEQ ID NO:767), Sequence A (SEQ ID NO:768), Sequence B (SEQ ID NO:769), Sequence C (SEQ ID NO:770), Sequence D (SEQ ID NO:771), Sequence E (SEQ ID NO:772), Sequence F (SEQ ID NO:773), Sequence G (SEQ ID NO:774), Sequence H (SEQ ID NO:775), Sequence I (SEQ ID NO:776), Sequence J (SEQ ID NO:777), Sequence K (SEQ ID NO:778), Sequence L (SEQ ID NO:779), Sequence M (SEQ ID NO:780), Sequence N (SEQ ID NO:781), Sequence O (SEQ ID NO:782), Sequence P (SEQ ID NO:783), and Sequence Q (SEQ ID NO:784).

If desired, the antagonist of IL-1R1 further comprises a polypeptide binding domain that has binding specificity for Tumor Necrosis Factor Receptor 1 (TNFR1, p55) and inhibits binding of Tumor Necrosis Factor Alpha (TNFα) to TNFR1.

Preferably, the antagonist of IL-1R1 binds human IL-1R1 with an affinity (KD) of about 300 nM to about 5 pM, as determined by surface plasmon resonance.

In more particular aspects, the invention relates to the use of an antagonist of Interleukin-1 Receptor Type 1 (IL-1R1) for the manufacture of a medicament for treating a respiratory disease, wherein said antagonist of IL-1R1 is a fusion protein or a conjugate comprising an antagonist of IL-1R1 moiety and a half-life extending moiety, wherein said antagonist of IL-1R1 moiety binds human IL-IR1 and inhibits binding of a ligand selected from the group consisting of Interleukin-1 (IL-1α) and Interleukin-1β (IL-1β) to human IL-1R1, and said half-life extending moiety is a polypeptide binding moiety that contains a binding site with binding specificity for a polypeptide that enhances serum half-life in vivo.

In some embodiments, the antagonist of IL-1R1 moiety is human Interleukin 1 receptor antagonist (IL-1ra) or a functional variant of human IL-1ra. In other embodiments, the antagonist of IL-1R1moiety is an immunoglobulin single variable domain that competes for binding to IL-1R1 with an anti-IL-1R1 dAb disclosed herein, or the antagonist of IL-1R1 moiety is an immunoglobulin single variable domain that comprises an amino acid sequence that has at least about 90% amino acid sequence identity with an amino acid sequence of a dAb disclosed herein.

The half-life extending moiety can be serum albumin or a fragment thereof, transferrin receptor or a transferrin-binding portion thereof, or an antibody or antibody fragment comprising a binding site for a polypeptide that enhances half-life in vivo. In particular embodiments, the half-life extending moiety is an immunoglobulin single variable domain that binds serum albumin and competes with an anti-serum albumin dAb disclosed herein for binding to serum albumin. In other embodiments, the half-life extending moiety is an immunoglobulin single variable domain that binds human serum albumin comprises the amino acid sequence of an anti-serum albumin dAb disclosed herein.

In more particular aspects, the invention relates to use of an antagonist of Interleukin-1 Receptor Type 1 (IL-1R1) for the manufacture of a medicament for treating a respiratory disease, wherein said antagonist of IL-1R1 comprises an immunoglobulin single variable domain that has binding specificity for human IL-1R1 and inhibits binding of a ligand selected from the group consisting of Interleukin-1α (IL-1α) and Interleukin-1β (IL-1β) to human IL-1R1, and a polyethylene glycol moiety. In one embodiment, the immunoglobulin single variable domain competes for binding to human IL-1R1 with an anti-IL-IR1 dAb disclosed herein. In another embodiment, the immunoglobulin single variable domain binds human serum albumin and comprises the amino acid sequence of an anti-serum albumin dAb disclosed herein.

The invention also relates to a pharmaceutical composition comprising an antagonist of IL-1R1 as described herein and a physiologically acceptable vehicle or carrier. In some embodiment, the pharmaceutical composition comprises a physiologically acceptable vehicle or carrier for parenteral administration (e.g., intravenous administration, subcutaneous administration). In other embodiment, the pharmaceutical composition comprises a physiologically acceptable vehicle or carrier for local administration (e.g., local administration to pulmonary tissue, such as by inhalation or intra-nasal administration. I

The invention also relates to a drug delivery device comprising a pharmaceutical composition of the invention. For example, the drug deliver device can be a parenteral delivery device, intravenous delivery device, intramuscular delivery device, intraperitoneal delivery device, transdermal delivery device, pulmonary delivery device, intraarterial delivery device, intrathecal delivery device, intraarticular delivery device, subcutaneous delivery device, intranasal delivery device, vaginal delivery device, and rectal delivery device. In particular embodiments the drug delivery device is selected from the group consisting of a syringe, a transdermal delivery device, a capsule, a tablet, a nebulizer, an inhaler, an atomizer, an aerosolizer, a mister, a dry powder inhaler, a metered dose inhaler, a metered dose sprayer, a metered dose mister, a metered dose atomizer, and a catheter.

The invention also relates to a method for treating a respiratory disease comprising administering to a subject in need thereof an effective amount of an antagonist of IL-1R1 as described herein.

### BRIEF DESCRIPTION OF THE DRAWINGS

FIGS. 1A and 1B are graphs showing the results of in vitro assays in which dAbs were tested for the ability to inhibit IL-1-induced IL-8 release from cultured MRC-5 cells (ATCC catalogue no. CCL-171). FIG. 1A shows a typical dose-response curve for an anti-IL-1R1 dAb referred to as DOM4-130 in such a cell assay. The ND₅₀ of DOM4-130 in the assay was approximately 500-1000 nM.

FIG. 1B shows a dose-response curve for anti-IL-1R1 dAbs referred to as DOM4-122 and DOM4-129 in such a cell assay. The ND50 values of both dAbs was about 1 μM.

FIGS. 2A and 2B are graphs showing the results of in vitro assays in which dAbs that underwent affinity maturation were tested for the ability to inhibit IL-1-induced IL-8 release from cultured MRC-5 cells (ATCC catalogue no. CCL-171).

FIG. 2A shows a dose-response curve for DOM4-130-3, which is an affinity matured variant of DOM4-130. The ND₅₀ for DOM4-130-3 in the assay was about 30 nM, compared to the ND50 for DOM4-130 which was 500-1000 nM (see FIG. 1A). FIG. 2B shows a dose-response curve for DOM4-130-46 and DOM4-130-51, which are affinity matured variants of DOM4-130, and for interleukin 1 receptor antagonist (IL-1ra). The ND₅₀ for DOM4-130-46 was about 1 nM in the assay, and the ND₅₀ for DOM4-130-51 about 300 pM).

FIG. 3 is a graph showing the results of in vitro assays in which dAbs that underwent affinity maturation were tested for the ability to inhibit IL-1-induced IL-8 release from cultured MRC-5 cells (ATCC catalogue no. CCL-171). FIG. 3 shows a dose-response curve for DOM4-122-6, DOM4-129-1, DOM4-122-23, and IL-1ra. DOM4-122-6 and DOM4-122-23 are affinity matured variants of DOM4-122, and DOM4-129-1 is an affinity matured variant of DOM4-129. Both DOM4-122-6 and DOM4-129-1 had an ND₅₀ of about 10 nM in the assay, and DOM4-122-23 had an ND₅₀ of approximately 1 nM in the assay.

FIG. 4 is a graph showing the results of an in vitro assay in which dAbs were tested for the ability to inhibit IL-1-induced IL-6 release in human whole blood. The results show that an IL-1R1 control antibody (a-IL-1R1Ab igG1), anti-IL-1R1 dAb (DOM4-130-54) and a dual specific format that contained an anti-IL-1R1 dAb and an anti-serum albumin dAb (DOM4-130-54/7h-8) each inhibited release of IL-6 in the assay, but that a dAb that binds serum albumin (DOM7h-8) did not.

FIG. 5 is a plot showing that an antagonist of IL-1R1 (IL1ra, a fusion protein in which IL-1ra was fused to an immunoglobulin single variable domain that binds mouse serum albumin) was efficacious in a subchronic model of tobacco smoke-induced (TS) chronic obstructive pulmonary disease (COPD) in C57BL/6 mice when administered intraperitoneally (10 mg/kg i.p.). The plot also shows that administration of IL-1R1 together with a dAb that binds TNFR1 was even more efficacious in a subchronic model of tobacco smoke-induced (TS) chronic obstructive pulmonary disease (COPD) in C57BL/6 mice when administered intraperitoneally. The plot shows the total number of cells present in bronchoalveolar lavage (BAL) of mice at completion of the study described in Example 2. The individual data points for each mouse in the study and the group averages (means; horizontal lines) are shown. The results show that antagonist of IL-1R1 reduced the number of cells in BAL by 58% compared to the untreated group (Veh), and that coadministration of the antagonist of IL-1R1 and an antagonist of TNFR1 reduced the number of cells in BAL by 88%. In contrast administration of ENBREL® (etanercept; Immunex Corporation) resulted in an increased number of total cells in BAL, although the increase was not statistically significant. TS, tobacco smoke-induced; Veh, vehicle; ns, not statistically significant.

FIG. 6 is a graph showing the levels of an immunoglobulin single variable domain that binds hen egg lysozyme (HEL-4) in the BAL at several time points after administration of the single variable domain to mice by pulmonary delivery. The graph shows HEL-4 was delivered efficiently into the deep lung. A dose related effect was observed. At 2 hours after administration, a maximum level of 700 ug/ml was detected in the lung with the 30 mg/kg dosing. The levels in the BAL are high for a prolonged period of time and declined gradually. The graph indicates that there was a slow release of HEL-4 into the surrounding tissues.

FIG. 7 is a graph showing the levels of HEL-4 in the serum at several time points after administration of the single variable domain to mice by pulmonary delivery. The graph shows that HEL-4 serum levels were detected in the 3 mg/kg and the 30 mg/kg dose groups. The serum levels showed a similar pattern as the BAL levels. There appeared to be a maximum level 2 hours after administration, followed by a slow decline. At 2 hours after administration, maximum levels of 3.5 μg/ml were detected in the serum with the 30 mg/kg dosing.

FIG. 8 is a graph showing the levels of IL-1ra (KINARET® (anakinra; Amgen)) in the BAL, lung tissue and serum at several time points after administration to mice by pulmonary delivery. The level in BAL was maximum at 1 hour after administration and was ˜11 μg/ml (˜2.75 μg in 0.25 ml of BAL fluid). The levels in the BAL were high for a prolonged period of time and show a gradual decline over 24 hrs. (>10-fold decline after 24 hrs). The levels in lung tissue was maximum at 1 hr and was ˜3.3 μg/ml. The levels in the lung were high for a prolonged period of time and show a gradual decline over 24 hrs. (>10-fold decline after 24 hrs). The level in the serum at 1 hr was lower than in BAL or lung tissue (260 ng/ml). At 5 hrs the levels in the serum was maximum (350 ng/ml). The levels in the serum show a slow decline and after 24 hrs there is only a 5-fold decline in the levels.

FIG. 9A-9 illustrates the amino acid sequences of several human dAbs that bind human IL-1R1. In some of the sequences, the amino acids of CDR1, CDR2 and CDR3 are underlined.

FIG. 10A-10BBB illustrates the nucleotide sequences of nucleic acids that encode the human dAbs shown in FIG. 9A-9X. In some of the sequences, the nucleotides encoding CDR1, CDR2 and CDR3 are underlined.

FIG. 11A is an alignment of the amino acid sequences of three VKS selected by binding to mouse serum albumin (MSA). The aligned amino acid sequences are from VKS designated MSA16, which is also referred to as DOM7m-16 (SEQ ID NO:723), MSA 12, which is also referred to as DOM7m-12 (SEQ ID NO:724), and MSA 26, which is also referred to as DOM7m-26 (SEQ ID NO:725).

FIG. 11B is an alignment of the amino acid sequences of six VKS selected by binding to rat serum albumin (RSA). The aligned amino acid sequences are from Vκs designated DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), and DOM7r-8 (SEQ ID NO:731).

FIG. 11C is an alignment of the amino acid sequences of six VKS selected by binding to human serum albumin (HSA). The aligned amino acid sequences are from Vκs designated DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), and DOM7h-7 (SEQ ID NO:737).

FIG. 11D is an alignment of the amino acid sequences of seven V_{H}s selected by binding to human serum albumin and a consensus sequence (SEQ ID NO:738). The aligned sequences are from V_{H}s designated DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), and DOM7h-27 (SEQ ID NO:745).

FIG. 11E is an alignment of the amino acid sequences of three VKS selected by binding to human serum albumin and rat serum albumin. The aligned amino acid sequences are from VKS designated DOM7h-8 (SEQ ID NO:746), DOM7r-13 (SEQ ID NO:747), and DOM7r-14 (SEQ ID NO:748).

FIG. 12 is an illustration of the amino acid sequences of VKS selected by binding to rat serum albumin (RSA). The illustrated sequences are from VKS designated DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753).

FIG. 13A-13B is an illustration of the amino acid sequences of the amino acid sequences of V_{H}s that bind rat serum albumin (RSA). The illustrated sequences are from V_{H}s designated DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), and DOM7r-33 (SEQ ID NO:767).

FIG. 14A is an illustration of the nucleotide sequence (SEQ ID NO:785) of a nucleic acid encoding human interleukin 1 receptor antagonist (IL-1ra) deposited in GenBank under accession number NM_{—}173842. The nucleic acid has an open reading frame starting at position 65.

FIG. 14B is an illustration of the amino acid sequence of human IL-1ra (SEQ ID NO:786) encoded by the nucleic acid shown in FIG. 15A (SEQ ID NO:785). The mature protein consists of 152 amino acid residues (amino acid residues 26-177 of SEQ ID NO:786).

FIG. 15 illustrates the amino acid sequences of several Camelid V_{HH}s that bind mouse serum albumin that are disclosed in WO 2004/041862. Sequence A (SEQ ID NO:768), Sequence B (SEQ ID NO:769), Sequence C (SEQ ID NO:770), Sequence D (SEQ ID NO:771), Sequence E (SEQ ID NO:772), Sequence F (SEQ ID NO:773), Sequence G (SEQ ID NO:774), Sequence H (SEQ ID NO:775), Sequence I (SEQ ID NO:776), Sequence J (SEQ ID NO:777), Sequence K (SEQ ID NO:778), Sequence L (SEQ ID NO:779), Sequence M (SEQ ID NO:780), Sequence N (SEQ ID NO:781), Sequence O (SEQ ID NO:782), Sequence P (SEQ ID NO:783), Sequence Q (SEQ ID NO:784).

### DETAILED DESCRIPTION OF THE INVENTION

As used herein, “interleukin-1 receptor type 1” (IL-1R1; CD121a) refers to naturally occurring or endogenous mammalian IL-1R1 proteins and to proteins having an amino acid sequence which is the same as that of a naturally occurring or endogenous corresponding mammalian IL-1R1 protein (e.g., recombinant proteins, synthetic proteins (i.e., produced using the methods of synthetic organic chemistry)). Accordingly, as defined herein, the term includes mature protein, polymorphic or allelic variants, and other isoforms of a IL-1R1 (e.g., produced by alternative splicing or other cellular processes), and modified or unmodified forms of the foregoing (e.g., lipidated, glycosylated). Naturally occurring or endogenous IL-1R1 include wild type proteins such as mature IL-1R1, polymorphic or allelic variants and other isoforms which occur naturally in mammals (e.g., humans, non-human primates). Such proteins can be recovered or isolated from a source which naturally produces IL-1R1, for example. These proteins and IL-1R1 proteins having the same amino acid sequence as a naturally occurring or endogenous corresponding IL-1R1, are referred to by the name of the corresponding mammal. For example, where the corresponding mammal is a human, the protein is designated as a human IL-1R1.

As used herein, “antagonist of interleukin-1 receptor type 1 (IL-1R1) moiety” refers to any compound (e.g., protein, polypeptide, peptide) that binds to IL-1R1 and inhibiting a function of IL-1R1 (e.g., inhibits binding of IL-1α and/or IL-1β to IL-1R1, inhibits signaling through IL-1R1 upon binding of IL-1βand/or IL-1β). An “antagonist of interleukin-1 receptor type 1” comprises an antagonists of interleukin-1 receptor type 1 (IL-1R1) moiety, and can comprise additional moieties if desired. An antagonist of interleukin-1 receptor type 1 (IL-1R1) moiety can be formatted into a variety of suitable structures as described herein.

As used herein, “antagonist of interleukin-1 receptor type 1 (IL-1R1)” refers to any compound (e.g., polypeptide) that can be administered to an individual to produce a beneficial therapeutic or diagnostic effect though binding to IL-1R1 and inhibiting a function of IL-1R1 in the individual. Preferred “antagonists of IL-1R1” comprise a peptide or polypeptide that binds IL-1R1 and inhibits a function of IL-1R1, such as interleukin-1 receptor antagonist (IL-1ra) and functional variants thereof, and antibodies that bind IL-1R1 and antigen-binding fragments thereof (e.g., dAbs). Antagonists of IL-1R1 include “conjugates,” such as a “covalent antagonist of IL-1R1 conjugate,” and a “noncovalent antagonists of IL-1R1 conjugate.” Antagonists of IL-1R1 also include fusion proteins, such as, an “antagonist of IL-1R1 fusion proteins.”

The “conjugates” comprise an antagonist of IL-1R1 moiety (e.g., IL-1ra or functional variant thereof, dAb) that is covalently or noncovalently bonded to a polypeptide binding moiety that contains a binding site (e.g., an antigen-binding site) with binding specificity for a polypeptide that enhances serum half-life in vivo (e.g., serum albumin). The antagonist of IL-1R1 moiety can be covalently or noncovalently bonded to a polypeptide binding moiety that contains a binding site (e.g., an antigen-binding site) that has binding specificity for a polypeptide that enhances serum half-life in vivo. The antagonist of IL-1R1 moiety can be covalently or noncovalently bonded to the polypeptide binding moiety directly or indirectly (e.g., through a suitable linker and/or noncovalent binding of complementary binding partners (e.g., biotin and avidin)). When complementary binding partners are employed, one of the binding partners can be covalently bonded to the antagonist of IL-1R1 moiety directly or through a suitable linker moiety, and the complementary binding partner can be covalently bonded to the polypeptide binding moiety directly or through a suitable linker moiety.

Preferably, the polypeptide binding moiety that has a binding site with binding specificity for a polypeptide that enhances serum half-live in vivo is an antigen-binding fragment of an antibody that binds serum albumin, (e.g., a V_{H}, V_{L}, V_{HH}). For example, the conjugate can be a “covalent antagonist of IL-1R1 conjugate” which refers to conjugates in which the antagonist of IL-1R1 moiety is covalently bonded to the antigen-binding fragment that binds serum albumin directly, or indirectly through a suitable linker moiety. The antagonist of IL-1R1 moiety can be bonded to the antigen-binding fragment at any suitable position, such as the amino-terminus, the carboxyl-terminus or through suitable amino acid side chains (e.g., the ε amino group of lysine). The antagonist of IL-1R1 can also be a “noncovalent antagonist of IL-1R1 conjugate,” which refers to conjugates in the antagonist of IL-1R1 moiety and the antigen-binding fragment of an antibody that binds serum albumin are noncovalently bonded. The antagonist of IL-1R1 moiety can be noncovalently bonded to the antigen-binding fragment directly (e.g., electrostatic interaction, hydrophobic interaction) or indirectly (e.g., through noncovalent binding of complementary binding partners (e.g., biotin and avidin), wherein one partner is covalently bonded to the antagonist of IL-1R1 moiety and the complementary binding partner is covalently bonded to the antigen-binding fragment). When complementary binding partners are employed, one of the binding partners can be covalently bonded to the antagonist of IL-1R1 moiety directly or through a suitable linker moiety, and the complementary binding partner can be covalently bonded to the antigen-binding fragment of an antibody that binds serum albumin directly or through a suitable linker moiety.

As used herein, “antagonist of IL-1R1 fusion” refers to a fusion protein that comprises an antagonist of interleukin-1 receptor type 1 (IL-1R1) moiety that is a peptide or polypeptide, and an antigen-binding fragment of an antibody that binds serum albumin. The peptide or polypeptide antagonist of interleukin-1 receptor type 1 (IL-1R1) moiety, and the antigen-binding fragment of an antibody that binds serum albumin are present as discrete parts (moieties) of a single continuous polypeptide chain.

As used herein, “interleukin 1 receptor antagonist” (IL-1ra) refers to naturally occurring or endogenous mammalian IL-1ra proteins and to proteins having an amino acid sequence which is the same as that of a naturally occurring or endogenous corresponding mammalian IL-1ra protein (e.g., recombinant proteins, synthetic proteins (i.e., produced using the methods of synthetic organic chemistry)). Accordingly, as defined herein, the term includes mature protein, polymorphic or allelic variants, and other isoforms of a IL-1ra (e.g., produced by alternative splicing or other cellular processes), and modified or unmodified forms of the foregoing (e.g., lipidated, glycosylated, PEGylated). Naturally occurring or endogenous IL-1ra include wild type proteins such as mature IL-1ra, polymorphic or allelic variants and other isoforms which occur naturally in mammals (e.g., humans, non-human primates). Such proteins can be recovered or isolated from a source which naturally produces IL-1ra, for example. These proteins and IL-1ra proteins having the same amino acid sequence as a naturally occurring or endogenous corresponding IL-1ra, are referred to by the name of the corresponding mammal. For example, where the corresponding mammal is a human, the protein is designated as a human IL-1ra.

“Functional variants” of IL-1ra include functional fragments, functional mutant proteins, and/or functional fusion proteins which can be produce using suitable methods (e.g., mutagenesis (e.g., chemical mutagenesis, radiation mutagenesis), recombinant DNA techniques). A “functional variant” antagonizes IL-1R1. Generally, fragments or portions of IL-1ra include those having a deletion and/or addition (i.e., one or more amino acid deletions and/or additions) of an amino acid (i.e., one or more amino acids) relative to the mature IL-1ra (such as N-terminal, C-terminal or internal deletions). Fragments or portions in which only contiguous amino acids have been deleted or in which non-contiguous amino acids have been deleted relative to mature IL-1ra are also envisioned.

A functional variant of human IL-1ra can have at least about 80%, or at least about 85%, or at least about 90%, or at least about 95%, or at least about 96%, or at least about 97%, or at least about 98%, or at least about 99% amino acid sequence identity with the mature 152 amino acid form of human IL-1ra and antagonize human Interleukin-1 type 1 receptor. (See, Eisenberg et al., Nature 343:341-346 (1990).) The variant can comprise one or more additional amino acids (e.g., comprise 153 or 154 or more amino acids). For example, the variant IL-1ra can have an amino acid sequence that consists of an amino-terminal methionine residue followed by residues 26 to 177 of SEQ ID NO:786. (KINERET® (anakinra), Amgen).

The phrase “immunoglobulin single variable domain” refers to an antibody variable region (V_{H}, V_{HH}, V_{L}) that specifically binds an antigen or epitope independently of other V regions or domains; however, as the term is used herein, an immunoglobulin single variable domain can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of the additional variable domains). “Immunoglobulin single variable domain” encompasses not only an isolated antibody single variable domain polypeptide, but also larger polypeptides that comprise one or more monomers of an antibody single variable domain polypeptide sequence. A “domain antibody” or “dAb” is the same as an “immunoglobulin single variable domain” polypeptide as the term is used herein. An immunoglobulin single variable domain polypeptide, as used herein refers to a mammalian immunoglobulin single variable domain polypeptide, preferably human, but also includes rodent (for example, as disclosed in WO 00/29004, the contents of which are incorporated herein by reference in their entirety) or camelid V_{HH} dabs. Camelid dabs are immunoglobulin single variable domain polypeptides which are derived from species including camel, llama, alpaca, dromedary, and guanaco, and comprise heavy chain antibodies naturally devoid of light chain: V_{HH}. V_{HH} molecules are about ten times smaller than IgG molecules, and as single polypeptides, they are very stable, resisting extreme pH and temperature conditions.

“Complementary” Two immunoglobulin domains are “complementary” where they belong to families of structures which form cognate pairs or groups or are derived from such families and retain this feature. For example, a V_{H} domain and a V_{L} domain of an antibody are complementary; two V_{H} domains are not complementary, and two V_{L} domains are not complementary. Complementary domains may be found in other members of the immunoglobulin superfamily, such as the V_{α} and V_{β} (or γ and δ) domains of the T-cell receptor. Domains which are artificial, such as domains based on protein scaffolds which do not bind epitopes unless engineered to do so, are non-complementary. Likewise, two domains based on (for example) an immunoglobulin domain and a fibronectin domain are not complementary.

“Domain” A domain is a folded protein structure which retains its tertiary structure independently of the rest of the protein. Generally, domains are responsible for discrete functional properties of proteins, and in many cases may be added, removed or transferred to other proteins without loss of function of the remainder of the protein and/or of the domain. By single antibody variable domain is meant a folded polypeptide domain comprising sequences characteristic of antibody variable domains. It therefore includes complete antibody variable domains and modified variable domains, for example in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain.

“Repertoire” A collection of diverse variants, for example polypeptide variants which differ in their primary sequence. A library used in the present invention will encompass a repertoire of polypeptides comprising at least 1000 members.

“Library” The term library refers to a mixture of heterogeneous polypeptides or nucleic acids. The library is composed of members, each of which have a single polypeptide or nucleic acid sequence. To this extent, library is synonymous with repertoire. Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. Preferably, each individual organism or cell contains only one or a limited number of library members. Advantageously, the nucleic acids are incorporated into expression vectors, in order to allow expression of the polypeptides encoded by the nucleic acids. In a preferred aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

“Antibody” An antibody (for example IgG, IgM, IgA, IgD or IgE) or fragment (such as a Fab, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria).

“Dual-specific ligand” A ligand comprising a first immunoglobulin single variable domain and a second immunoglobulin single variable domain as herein defined, wherein the variable regions are capable of binding to two different antigens or two epitopes on the same antigen which are not normally bound by a monospecific immunoglobulin. For example, the two epitopes may be on the same hapten, but are not the same epitope or sufficiently adjacent to be bound by a monospecific ligand. The dual specific ligands according to the invention are composed of variable domains which have different specificities, and do not contain mutually complementary variable domain pairs which have the same specificity. Dual-specific ligands and suitable methods for preparing dual-specific ligands are disclosed in WO 2004/058821, WO 2004/003019, and WO 03/002609, the entire teachings of each of these published international applications are incorporated herein by reference.

“Antigen” A molecule that is bound by a ligand according to the present invention. Typically, antigens are bound by antibody ligands and are capable of raising an antibody response in vivo. It may be a polypeptide, protein, nucleic acid or other molecule. Generally, the dual specific ligands according to the invention are selected for target specificity against a particular antigen. In the case of conventional antibodies and fragments thereof, the antibody binding site defined by the variable loops (L1, L2, L3 and H1, H2, H3) is capable of binding to the antigen.

“Epitope” A unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation.

“Universal framework” A single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, (1987) J. Mol. Biol. 196:910-917. The invention provides for the use of a single framework, or a set of such frameworks, which has been found to permit the derivation of virtually any binding specificity though variation in the hypervariable regions alone.

“Half-life” The time taken for the serum concentration of the ligand to reduce by 50%, in vivo, for example due to degradation of the ligand and/or clearance or sequestration of the ligand by natural mechanisms. The ligands of the invention are stabilised in vivo and their half-life increased by binding to molecules which resist degradation and/or clearance or sequestration. Typically, such molecules are naturally occurring proteins which themselves have a long half-life in vivo. The half-life of a ligand is increased if its functional activity persists, in vivo, for a longer period than a similar ligand which is not specific for the half-life increasing molecule. Thus, a ligand specific for HSA and a target molecule is compared with the same ligand wherein the specificity for HSA is not present, that it does not bind HSA but binds another molecule. For example, it may bind a second epitope on the target molecule. Typically, the half life is increased by 10%, 20%, 30%, 40%, 50% or more. Increases in the range of 2×, 3×, 4×, 5×, 10Δ, 20×, 30×, 40×, 50× or more of the half life are possible. Alternatively, or in addition, increases in the range of up to 30×, 40×, 50×, 60×, 70×, 80×, 90×, 100×, 150× of the half life are possible.

As used herein, the term “antagonist of Tumor Necrosis Factor Receptor 1 (TNFR1)” refers to an agent (e.g., a molecule, a compound) which binds TNFR1 and can inhibit a (i.e., one or more) function of TNFR1. For example, an antagonist of TNFR1 can inhibit the binding of TNFα to TNFR1 and/or inhibit signal transduction mediated through TNFR1. Accordingly, TNFR1-mediated processes and cellular responses (e.g., TNFα-induced cell death in a standard L929 cytotoxicity assay) can be inhibited with an antagonist of TNFR1. An antagonist of TNFR1 can be, for example, a small organic molecule, natural product, protein, peptide or peptidomimetic. Antagonists of TNFR1 can be identified, for example, by screening libraries or collections of molecules, such as, the Chemical Repository of the National Cancer Institute, as described herein or using other suitable methods. Preferred antagonists of TNFR1 are antibodies and antigen-binding fragments of antibodies (e.g., dAb monomers).

The invention relates to use of an antagonist of IL-1R1 for the manufacture of a medicament preventing, treating, or mitigating lung inflammation or a respiratory disease, such as those described herein (e.g., chronic obstructive respiratory disease (COPD), asthma). Preferably, the medicament is for pulmonary delivery. The invention also relates to methods for preventing, treating, or mitigating lung inflammation or a respiratory disease comprising administering to a subject in need thereof a therapeutically effective amount of antagonist of IL-1R1. Preferably, the method comprises administering the antagonist of IL-1R1 via pulmonary delivery. The invention also relates to pharmaceutical compositions for preventing, treating, or mitigating lung inflammation or a respiratory disease, such as those described herein (e.g., chronic obstructive respiratory disease, asthma), comprising as an active ingredient an antagonist of IL-1R1. Preferably, the pharmaceutical composition is for pulmonary delivery.

As described herein, polypeptide antagonists of IL-1R1 can be administered to a subject in need thereof to prevent, treat or mitigate lung inflammation, a respiratory disease or the symptoms thereof. For example, described herein are the results of a study evaluating the efficacy of an antagonist of IL-1R1 (IL-R1ra/anti-SA) in a mouse sub-chronic model of COPD induced by tobacco smoke. The results of this study revealed that the antagonists of IL-1R1 (IL-1ra/SA) was efficacious and significantly reduced the amount of inflammatory cells in lung of treated animals compared to control animals (reduced the number of total cells, macrophages, polymorphic nuclear cells, lymphocytes and eosinophils recovered in bronchioalveolar lavage (BAL)). The results indicate the administration of other antagonists of IL-1R1, such as antagonists that comprise an immunoglobulin variable domain the binds IL-1R1 and inhibits binding of a ligand (e.g., IL-1α, Il-1β) to IL-1R1, can also be administered to efficaciously treat lung inflammation, a respiratory disease or the symptoms thereof.

Antagonists of IL-1R1, such as peptide and polypeptide antagonists, can be delivered to a subject in need thereof in therapeutically effective amounts by pulmonary delivery (e.g., by inhalation). For example, as shown herein, pulmonary delivery of a dAb (HEL4) by inhalation resulted in efficient delivery of the dAb to the deep lung, as assessed by the amount of dAb recovered in BAL collected up to 24 hours after the dAb was delivered. Accordingly, as shown by the studies described herein, respiratory diseases can be treated by administering an antagonist of IL-1R1 locally to pulmonary tissue. This approach provides several advantages, such as minimizing or eliminating systemic side effects (e.g, increased incidence of infection) that have been reported to be associated with systemic administration of agents that antagonize IL-1R1, such as KINERET (e.g., anakinra, Amgen).

### Antagonists of TNFR1 for Treating, Suppressing or Preventing Lung Inflammation and Respiratory Diseases.

The invention relates to methods for treating, suppressing or preventing lung inflammation and/or a respiratory disease comprising administering to a subject (e.g., a mammal, a human) in need thereof an effective amount of an antagonist of IL-1R1. The invention also relates to the use of an antagonist of IL-1R1 for the manufacture of a medicament for treating, suppressing or preventing lung inflammation and/or respiratory disease, and to a pharmaceutical composition for treating, suppressing or preventing lung inflammation and/or respiratory disease comprising an antagonist of IL-1R1 as an active ingredient. Antagonists of TNFR1 suitable for use in the invention are described in detail herein and include small molecules, new chemical entities, IL-1ra and functional variants thereof, dAb monomers, and the like.

The invention comprises methods of administering antagonists of IL-1R1 for in in vivo therapeutic and prophylactic applications, in vivo diagnostic applications and the like. Therapeutic and prophylactic uses of antagonists of IL-1R1 comprise administering an effective amount of antagonists of IL-1R1 to a recipient mammal or subject, such as a human.

For example, the antagonists of IL-1R1 will typically find use in preventing, suppressing or treating lung inflammation and/or respiratory diseases, such as a condition in which lung inflammation is a symptom or part of the pathology, acute respiratory diseases, chronic respiratory diseases, acute inflammatory respiratory diseases and chronic inflammatory respiratory diseases. For example, the antagonists of IL-1R1 can be administered to treat, suppress or prevent lung inflammation, chronic obstructive respiratory disease (e.g., chronic bronchitis, chronic obstructive bronchitis, emphysema), asthma (e.g., steroid resistant asthma), pneumonia (e.g., bacterial pneumonia, such as Staphylococcal pneumonia), hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease (IPD), influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X (eosinophilic granuloma), pulmonary hypertension, pulmonary nocardiosis, pulmonary tuberculosis, pulmonary veno-occlusive disease, rheumatoid lung disease, sarcoidosis, Wegener's granulomatosis, and non-small cell lung carcinoma.

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Advantageously, dual- or multi-specific ligands may be used to target IL-1R1 and other molecules in therapeutic situations in the body of an organism. The invention therefore provides a method for synergising the activity of two or more binding domains (e.g., dAbs) wherein one domain binds IL-1R1 or other target in pulmonary tissue, and the other domain binds a cytokine, receptor or other molecules, comprising administering a dual- or multi-specific ligand capable of binding to said two or more molecules (e.g., IL-1R1 and a cytokine). For example, this aspect of the invention relates to combinations of V_{H} domains and V_{L} domains, V_{H} domains only and V_{L} domains only.

Synergy in a therapeutic context may be achieved in a number of ways. For example, target combinations may be therapeutically active only if both targets are targeted by the ligand, whereas targeting one target alone is not therapeutically effective. In another embodiment, one target alone may provide some therapeutic effect, but together with a second target the combination provides a synergistic increase in therapeutic effect (more than an additive effect).

Animal model systems which can be used to screen the effectiveness of the antagonists of IL-1R1 in preventing, suppressing or treating lung inflammation or a respiratory disease are available. For example, suitable animal models of respiratory disease include models of chronic obstructive respiratory disease (see, Groneberg, D A et al., Respiratory Research 5:18 (2004)), and models of asthma (see, Coffman et al., J. Exp. Med. 201(12):1875-1879 (2001). Preferably, the antagonist of IL-1R1 is efficacious in a mouse tobacco smoke-induced model of chronic obstructive respiratory disease (e.g., the subchronic model disclosed herein) or a suitable primate model of asthma or chronic obstructive respiratory disease. More preferably, the antagonist of IL-1R1 is efficacious in a mouse tobacco smoke-induced model of chronic obstructive respiratory disease (e.g., the subchronic model disclosed herein) (See, also, Wright and Churg, Chest, 122:301-306 (2002)). For example, administering an effective amount of the ligand can reduce, delay or prevent onset of the symptoms of COPD in the model, as compared to a suitable control. The prior art does not disclose using antagonists of IL-1R1 in these models, or that they would be efficacious.

Generally, the present antagonists of IL-1R1 will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition). A variety of suitable formulations can be used, including extended release formulations.

The antagonists of IL-1R1 may be used as separately administered compositions or in conjunction with other agents. These can include various drugs, such as phosphodiesterase inhibitors (e.g., inhibitors of phosphodiesterase 4), bronchodilators (e.g., beta2-agonists, anticholinergerics, theophylline), short-acting beta-agonists (e.g., albuterol, salbutamol, bambuterol, fenoterol, isoetherine, isoproterenol, levalbuterol, metaproterenol, pirbuterol, terbutaline and tornlate), long-acting beta-agonists (e.g., formoterol and salmeterol), short acting anticholinergics (e.g., ipratropium bromide and oxitropium bromide), long-acting anticholinergics (e.g., tiotropium), theophylline (e.g. short acting formulation, long acting formulation), inhaled steroids (e.g., beclomethasone, beclometasone, budesonide, flunisolide, fluticasone propionate and triamcinolone), oral steroids (e.g., methylprednisolone, prednisolone, prednisolon and prednisone), combined short-acting beta-agonists with anticholinergics (e.g., albuterol/salbutamol/ipratopium, and fenoterol/ipratopium), combined long-acting beta-agonists with inhaled steroids (e.g., salmeterol/fluticasone, and formoterol/budesonide) and mucolytic agents (e.g., erdosteine, acetylcysteine, bromheksin, carbocysteine, guiafenesin and iodinated glycerol), cylcosporine, antibiotics, antivirals, methotrexate, adriamycin, cisplatinum, and immunotoxins.

Pharmaceutical compositions can include “cocktails” of various cytotoxic or other agents in conjunction with the antagonist of IL-1R1, or even combinations of antagonists of IL-1R1 having different specificities, such as antagonists of IL-1R1 (e.g., a dAb) selected using different target epitopes, whether or not they are pooled prior to administration.

The antagonists of IL-1R1 can be administered and/or formulated together with one or more additional therapeutic or active agents. When an antagonist of IL-1R1 is administered with an additional therapeutic agent, the antagonist of IL-1R1 can be administered before, simultaneously with or subsequent to administration of the additional agent. Generally, the antagonist of IL-1R1 and additional agent are administered in a manner that provides an overlap of therapeutic effect. In particular embodiments, the antagonist of IL-1R1 is administered and/or formulated together with an antagonist of TNFR1.

The compositions containing an antagonist of IL-1R1 or a cocktail thereof can be administered for prophylactic and/or therapeutic treatments. In certain therapeutic applications, an amount that is sufficient to accomplish at least partial inhibition, suppression, modulation, killing, or some other measurable parameter, of a population of selected cells is defined as a “therapeutically-effective dose”. For example, for treating lung inflammation and/or a respiratory disease, a sputum-inhibiting amount, a bronchial biopsy inflammation-inhibiting amount, a dyspnoea-inhibiting amount, a forced expiratory volume in one second (FEV (1)) increasing amount, an improvement in health status increasing amount, as quantified in a suitable questionnaire such as the St. George's Respiratory Questionnaire (e.g., an improvement score of 4 points).

Amounts needed to achieve these effects will depend upon the severity of the disease and the general state of the patient's own immune system, but generally range from 0.005 to 10.0 mg of antagonist of IL-1R1 per kilogram of body weight, with doses of 0.05 to 2.0 mg/kg/dose being more commonly used.

For prophylactic applications, compositions containing the antagonist of IL-1R1 or cocktails thereof may also be administered in similar or slightly lower dosages, to prevent, inhibit or delay onset of disease (e.g., to sustain remission or quiescence, or to prevent acute phase). The skilled clinician will be able to determine the appropriate dosing interval to treat, suppress or prevent disease. When an antagonist of IL-1R1 is administered to treat, suppress or prevent lung inflammation or a respiratory disease, it can be administered up to four times per day, twice weekly, once weekly, once every two weeks, once a month, or once every two months, at a dose off, for example, about 10 μg/kg to about 80 mg/kg, about 100 μg/kg to about 80 mg/kg, about 1 mg/kg to about 80 mg/kg, about 1 mg/kg to about 70 mg/kg, about 1 mg/kg to about 60 mg/kg, about 1 mg/kg to about 50 mg/kg, about 1 mg/kg to about 40 mg/kg, about 1 mg/kg to about 30 mg/kg, about 1 mg/kg to about 20 mg/kg, about 1 mg/kg to about 10 mg/kg, about 10 μg/kg to about 10 mg/kg, about 10 μg/kg to about 5 mg/kg, about 10 μg/kg to about 2.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg. In particular embodiments, the antagonist of IL-1R1 is administered to treat, suppress or prevent lung inflammation or a respiratory disease each day, every two days, once a week, once every two weeks or once a month at a dose of about 10 μg/kg to about 10 mg/kg (e.g., about 10 μg/kg, about 100 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg). The antagonist of IL-1R1 can also be administered to treat, suppress or prevent lung inflammation or a respiratory disease at a daily dose or unit dose of about 10 mg, about 9 mg, about 8 mg, about 7 mg, about 6 mg, about 5 mg, about 4 mg, about 3 mg, about 2 mg or about 1 mg.

Treatment or therapy performed using the antagonist of IL-1R1 described herein is considered “effective” if one or more symptoms are reduced (e.g., by at least 10% or at least one point on a clinical assessment scale), relative to such symptoms present before treatment, or relative to such symptoms in an individual (human or model animal) not treated with such composition or other suitable control. Symptoms will vary depending upon the disease or disorder targeted, but can be measured by an ordinarily skilled clinician or technician. Such symptoms can be measured, for example, by monitoring one or more physical indicators of the disease or disorder (e.g., cellular infiltrate in lung tissue, production of sputum, cellular infiltrate in sputum, dyspnoea, exercise tolerance, spirometry (e.g., forced vital capacity (FVC), force expiratory volume in one second (FEV (1), FEV (1)/FVC), rate or severity of disease exacerbation, or by an accepted clinical assessment scale, for example, the St. George's Respiratory Questionnaire. Suitable clinical assessment scales include, for example, the severity of air flow obstruction according to FEV (1) (Clinical Guideline 12, Chronic Obstructive Respiratory disease, Management of Chronic Obstructive Pulmonary Disease in Adults in Primary and Secondary Care, National Institute for Clinical Excellence, London (2004)), Peak Expiratory Flow (PEF) (British Guideline on the Management of Asthma, British Thoracic Society, Scottish Intercollegiate Guidelines Network, Revised Edition (2004)), COPD stage according to the American Thoracic Society (ATS) standard (Am. J. Respir. Crit. Care Med., 152:S77-S120 (1995), asthma impairment class according to the ATS standard (Am. Rev. Respi. Dis., 147:1056-1061 (1993), or other accepted clinical assessment scale as known in the field. A sustained (e.g., one day or more, preferably longer) reduction in disease or disorder symptoms by at least 10% or by one or more points on a given clinical scale is indicative of “effective” treatment. Similarly, prophylaxis performed using a composition as described herein is “effective” if the onset or severity of one or more symptoms is delayed, reduced or abolished relative to such symptoms in a similar individual (human or animal model) not treated with the composition.

A composition containing an antagonist of IL-1R1 according to the present invention may be utilised in prophylactic and therapeutic settings to aid in the alteration, inactivation, killing or removal of a select target cell population in a mammal. For example, such compositions can be used to reduce levels of inflammatory cells in lung and/or inhibit cell infiltration of the lung.

The antagonists of IL-1R1 can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate. The antagonist of IL-1R1 can be lyophilised to form a dry powder for inhalation, and administered in that form.

The route of administration of pharmaceutical compositions according to the invention may be any of those commonly known to those of ordinary skill in the art. The administration can be by any appropriate mode, including parenterally, intravenously, intramuscularly, intraperitoneally, transdermally, via the pulmonary route, or by direct infusion with a catheter. The dosage and frequency of administration will depend on the age, sex and condition of the patient, concurrent administration of other drugs, counterindications and other parameters to be taken into account by the clinician. Administration can be local (e.g., local delivery to the lung by pulmonary administration, e.g., intranasal administration) or systemic as indicated.

In particular embodiments, an antagonist of IL-1R1 is administered via pulmonary delivery, such as by inhalation (e.g., intrabronchial, intranasal or oral inhalation, intranasal drops) or by systemic delivery (e.g., parenteral, intravenous, intramuscular, intraperitoneal, subcutaneous). In preferred embodiments, the antagonist of IL-1R1 is administered to a subject via pulmonary administration, such as inhalation or intranasal administration (e.g., intrabronchial, intranasal or oral inhalation, intranasal drops). For inhalation, the antagonist of IL-1R1 can be administered with the use of a nebulizer, inhaler, atomizer, aerosolizer, mister, dry powder inhaler, metered dose inhaler, metered dose sprayer, metered dose mister, metered dose atomizer, or other suitable delivery device.

The invention relates to a method for treating, suppressing or preventing lung inflammation or a respiratory disease, comprising administering to a subject in need thereof an effective amount of an antagonist of IL-1R1. In some embodiments, the effective amount administered does not exceed about 10 mg/kg/day, and preferably the level of inflammatory cells in the lung is reduced relative to pretreatment levels, or recruitment of inflammatory cells into the lung is inhibited relative to pretreatment levels. The level of inflammatory cells in the lung or recruitment of inflammatory cells into the lung can be reduced or inhibited relative to pretreatment levels by at least about 30%, at least about 40%, at least about 50%, at least about 60%, at least about 70%, at least about 80%, at least about 90%, or at least about 95%.

The level of inflammatory cells in the lung or recruitment of inflammatory cells into the lung can be reduced or inhibited relative to pretreatment levels with p<0.05 or p<0.001, in some embodiments. Preferably, statistical analysis and significance is determined using the methods described herein.

Levels of cells (e.g., inflammatory cells) in the lung can be assessed using any suitable method, such as total or differential cell counts (e.g., macrophage cell count, neutrophil cell count, eosinophil cell count, lymphocyte cell count, epithelial cell count) in BAL, sputum or biopsy (e.g., bronchial biopsy, lung biopsy).

In some embodiments, the methods described herein are employed for treating, suppressing or preventing chronic obstructive respiratory disease (e.g., chronic bronchitis, chronic obstructive bronchitis, emphysema), asthma (e.g., steroid resistant asthma), pneumonia (e.g., bacterial pneumonia, such as Staphylococcal pneumonia), or lung inflammation.

The invention also relates to the use of an antagonist of IL-1R1, as described herein, for the manufacture of a medicament or formulation for treating lung inflammation or a respiratory disease described herein. The medicament can be for systemic administration and/or local administration to pulmonary tissue.

The invention provides methods of treating a respiratory disease in which a therapeutically effective amount of an antagonist of IL-1R1 (e.g., IL-1ra, dAb, ligand) is administered systemically to a subject in need. In some embodiments, the method further comprises administering a therapeutically effective amount of an antagonist of TNFR1 to the subject. The antagonist of TNFR1 can be administered by any suitable method, such as by pulmonary administration or systemic administration.

### Antagonists of IL-1R1

Antagonists of IL-1R1 suitable for use in the invention include, for example, small molecules, proteins, polypeptides (e.g., fusion proteins), peptides and conjugates that bind IL-1R1 and inhibit a function of IL-1R1 (e.g., binding of IL-1α and/or IL-1β; inhibit signaling upon binding of IL-1α and/or IL-1β). As described herein, an antagonist of IL-1R1 suitable for use in the invention comprise an antagonist of IL-1R1 moiety, that can be formatted into a variety of suitable structures. For example, antagonists of IL-1R1 include proteins or polypeptides that comprise IL-1ra or functional variants of IL-1 ra, and proteins, polypeptides and peptides that comprise a binding domain that has a binding site with binding specificity for IL-1R1 and inhibits a function of IL-1R1. Preferably, the binding domain that has a binding site with binding specificity for IL1-R1 and inhibits a function of IL-1R1 is an antibody that bind IL-1R1 or an antigen-binding fragment thereof, such as, Fab fragment, Fab′ fragment, F(ab′)₂ fragment, Fv fragment (e.g., single chain Fv (scFv), disulfide bonded Fv fragment), domain antibody (dAbs; single V_{H}, single V_{κ}, single V_{λ}), Camelid V_{HH} and the like. The binding domain can comprises one or more complementarity determining regions (CDRs) of an immunoglobulin single variable domain that has binding specificity for IL-1R1 in a suitable format, such that the binding domain has binding specificity for IL-1R1. For example, the CDRs can be grafted onto a suitable protein scaffold or skeleton, such as an affibody, an SpA scaffold, an LDL receptor class A domain, or an EGF domain. The binding domain can also be a protein domain comprising a binding site for IL-1R1, e.g., a protein domain is selected from an affibody, an SpA domain, an LDL receptor class A domain an EGF domain, an avimer (see, e.g., U.S. Patent Application Publication Nos. 2005/0053973, 2005/0089932, 2005/0164301).

In some embodiments, the antagonist of IL-1R1 comprises a non-immunoglobulin binding moiety that has binding specificity for IL-1R1 and inhibits a function of IL-1R1, wherein the non-immunoglobulin binding moiety comprises one, two or three of the CDRs of a V_{H}, V_{L} or V_{HH} that binds IL-1R1 and a suitable scaffold. In certain embodiments, the non-immunoglobulin binding moiety comprises CDR3 but not CDR1 or CDR2 of a V_{H}, V_{L} or V_{HH} that binds IL-1R1 and a suitable scaffold. In other embodiments, the non-immunoglobulin binding moiety comprises CDR1 and CDR2, but not CDR3 of a V_{H}, V_{L} or V_{HH} that binds IL-1R1 and a suitable scaffold. In other embodiments, the non-immunoglobulin binding moiety comprises CDR1, CDR2 and CDR3 of a V_{H}, V_{L} or V_{HH} that binds IL-1R1 and a suitable scaffold. In other embodiments, the antagonist of IL-1R1 comprises only CDR3 of a V_{H}, V_{L} or V_{HH} that binds IL-1R1. Preferably, the CDR or CDRs of the antagonist of IL-1R1 of these embodiments is a CDR or CDRs of a V_{H}, or V_{L} that binds IL-1R1 described herein.

Suitable antagonists of IL-1R1 for use in the invention also include conjugates, such as a covalent antagonist of IL-1R1 conjugates, and a noncovalent antagonists of IL-1R1 conjugates, and fusion proteins, such as, an antagonist of IL-1R1 fusion, as defined herein. For example, the antagonist of IL-1R1 can be a fusion protein that that comprise IL-1ra, a functional variant of IL-1ra, an antibody that bind IL-1R1, an antigen-binding fragment of an antibody that binds IL-1R1 (e.g., a dAb), and/or a non-immunoglobulin binding moiety that has binding specificity for IL-1R1.

Preferred antagonists of IL-1R1 are polypeptides that comprise IL-1ra or functional variants of IL-1ra, and polypeptides that comprise a dAb that binds IL-1R1 and inhibits a function of IL-1R1.

Antibodies and Antibody Portions that Bind IL-1R1

The antagonist of IL-1R1 can comprise an (i.e., one or more) antibody or antigen-binding fragment of an antibody that binds IL-1R1 and inhibits function of IL-1R1. For example, the antibody or antigen-binding fragment thereof can bind IL-1R1 and inhibiting binding of a ligand (e.g., IL-1α, IL-1β, IL-1ra, or any combination of the foregoing) to the receptor, or inhibit IL-1R1 mediated signaling upon binding of a ligand (e.g., IL-1α, IL-1β). The antibody or antigen-binding fragment can have binding specificity for IL-1R1 of an animal to which the antagonist of IL-1R1 will be administered. Preferably, the antibody or antigen-binding fragment has binding specificity for human IL-1R1. However, veterinary applications are contemplated and the antibody or antigen-binding fragment can have binding specificity for IL-1R1 from a desired animal, for example IL-1R1 from dog, cat, horse, cow, chicken, sheep, pig, goat, deer, mink, and the like. In some embodiments, the antibody or antigen-binding fragment has binding specificity for IL-1R1 from more than one species. Such antibodies or antigen-binding fragment provide the advantage of allowing preclinical and clinical studies to be designed and executed using the same antagonist of IL-1R1, and obviate the need to conduct preclinical studies with a suitable surrogate antagonist of IL-1R1.

Antibodies and antigen-binding fragments thereof which bind a desired IL-1R1 (e.g., human IL-1R1) can be selected from a suitable collection of natural or artificial antibodies or raised against an appropriate immunogen in a suitable host. For example, antibodies can be raised by immunizing a suitable host (e.g., mouse, human antibody-transgenic mouse, rat, rabbit, chicken, goat, non-human primate (e.g., monkey)) with IL-1R1 (e.g., isolated or purified human IL-1R1) or a peptide of IL-1R1 (e.g., a peptide comprising at least about 8, 9, 10, 11, 12, 15, 20, 25, 30, 33, 35, 37, or 40 amino acid residues). Antibodies and antigen-binding fragments that bind IL-1R1 can also be selected from a library of recombinant antibodies or antigen-binding fragments, such as a phage display library. Such libraries can contain antibodies or antigen-binding fragments of antibodies that contain natural or artificial amino acid sequences. For example, the library can contain Fab fragments which contain artificial CDRs (e.g., random amino acid sequences) and human framework regions. (See, for example, U.S. Pat. No. 6,300,064 (Knappik, et al.).) In other examples, the library contains scFv fragments or dAbs (single V_{H}, single V_{κ} or single V_{λ}) with sequence diversity in one or more CDRs. (See, e.g., WO 99/20749 (Tomlinson and Winter), WO 03/002609 A2 (Winter et al.), WO 2004/003019A2 (Winter et al.).)

Antigen-binding fragments of antibodies that are suitable for use in the invention include, for example, Fab fragments, Fab′ fragments, F(ab′)₂ fragments, Fv fragments (including single chain Fv (scFv) and disulfide bonded Fv), a single variable domain (V_{H}, V_{L}, V_{HH}). Such antigen-binding fragments can be produced using any suitable method, such as by proteolysis of an antibody using pepsin, papain or other protease having the requisite cleavage specificity, or using recombinant techniques. For example, Fv fragments can be prepared by digesting an antibody with a suitable protease or using recombinant DNA technology. For example, a nucleic acid can be prepared that encodes a light chain variable region and heavy chain variable region that are connected by a suitable peptide linker, such as a chain of two to about twenty Glycyl residues or (Gly₄Ser)ₙ, where n=from 1 to 8, e.g., 1, 2, 3, 4, 5, 6, 7 or 8. The nucleic acid can be introduced into a suitable host (e.g., E. coli) using any suitable technique (e.g., transfection, transformation, infection), and the host can be maintained under conditions suitable for expression of a single chain Fv fragment. A variety of antigen-binding fragments of antibodies can be prepared using antibody genes in which one or more stop codons have been introduced upstream of the natural stop site. For example, an expression construct encoding a F(ab′)₂ portion of an immunoglobulin heavy chain can be designed by introducing a translation stop codon at the 3′ end of the sequence encoding the hinge region of the heavy chain. The antagonist of IL-1R1 can comprise the individual heavy and light chains of antibodies that bind IL-1R1 or portions of the individual chains that bind IL-1R1 (e.g., a single V_{H}, V_{κ} or V_{λ}).

Suitable antibodies and antigen-binding fragments thereof that bind IL-1R1 include, for example, human antibodies and antigen-binding fragments thereof, humanized antibodies and antigen-binding fragments thereof, chimeric antibodies and antigen-binding fragments thereof, rodent (e.g., mouse, rat) antibodies and antigen-binding fragments thereof, and Camelid antibodies and antigen-binding fragments thereof. In certain embodiments, the antagonist of IL-1R1 comprises a Camelid V_{HH} that binds IL-1R1. Camelid V_{HH}s are immunoglobulin single variable domain polypeptides which are derived from heavy chain antibodies that are naturally devoid of light chains. Such antibodies occur in Camelid species including camel, llama, alpaca, dromedary, and guanaco. V_{HH} molecules are about ten times smaller than IgG molecules, and as single polypeptides, are very stable and resistant to extreme pH and temperature conditions.

If antibodies are prepared by immunization, preparation of the immunizing antigen, and polyclonal and monoclonal antibody production can be performed using any suitable technique. A variety of methods have been described. (See, e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991).) When a monoclonal antibody is desired, a hybridoma can be produced by fusing suitable cells from an immortal cell line (e.g., a myeloma cell line such as SP2/0, P3X63Ag8.653 or a heteromyeloma) with antibody-producing cells. Antibody-producing cells can be obtained from the peripheral blood or, preferably the spleen or lymph nodes, of humans, human-antibody transgenic animals or other suitable animals immunized with the antigen of interest. Cells that produce antibodies of human origin (e.g., a human antibody) can be produced using suitable methods, for example, fusion of a human antibody-producing cell and a heteromyeloma or trioma, or immortalization of an activated human B cell via infection with Epstein Barr virus. (See, e.g., U.S. Pat. No. 6,197,582 (Trakht); Niedbala et al., Hybridoma, 17:299-304 (1998); Zanella et al., J Immunol Methods, 156:205-215 (1992); Gustafsson et al., Hum Antibodies Hybridomas, 2:26-32 (1991).) The fused or immortalized antibody-producing cells (hybridomas) can be isolated using selective culture conditions, and cloned by limiting dilution. Cells which produce antibodies with the desired specificity can be identified using a suitable assay (e.g., ELISA).

Antibodies also can be prepared directly (e.g., synthesized or cloned) from an isolated antigen-specific antibody producing cell (e.g., a cell from the peripheral blood or, preferably the spleen or lymph nodes determined to produce an antibody with desired specificity), of humans, human-antibody transgenic animals or other suitable animals immunized with the antigen of interest (see, e.g., U.S. Pat. No. 5,627,052 (Schrader)).

When the antagonist of IL-1R1 is for administration to a human, any antibody or antigen-binding fragment of an antibody that is part of the antagonists of IL-1R1 (e.g., an antibody or antigen-binding fragment thereof that binds IL-1R1 (e.g., human IL-1R1) or serum albumin (e.g., human serum albumin)) can be a human, humanized or chimeric antibody or an antigen-binding fragment of such an antibody. These types of antibodies and antigen-binding fragments are less immunogenic or non-immunogenic in humans and provide well-known advantages. For example, antagonists of IL-1R1 that comprise an antigen-binding fragment of a human, humanized or chimeric antibody can be administered repeatedly to a human with less or no loss of efficacy (compared with other fully immunogenic antibodies) due to the elaboration of human antibodies that bind to the antagonist of IL-1R1. When the antagonist of IL-1R1 is intended for veterinary administration, analogous antibodies or antigen-binding fragments can be used. For example, CDRs from a murine or human antibody can be grafted onto framework regions from a desired animal, such as a horse or cow.

Human antibodies and nucleic acids encoding same can be obtained, for example, from a human or from human-antibody transgenic animals. Human-antibody transgenic animals (e.g., mice) are animals that are capable of producing a repertoire of human antibodies, such as XENOMOUSE (Abgenix, Fremont, Calif.), HUMAB-MOUSE, KIRIN TC MOUSE or KM-MOUSE (MEDAREX, Princeton, N.J.). Generally, the genome of human-antibody transgenic animals has been altered to include a transgene comprising DNA from a human immunoglobulin locus that can undergo functional rearrangement. An endogenous immunoglobulin locus in a human-antibody transgenic animal can be disrupted or deleted to eliminate the capacity of the animal to produce antibodies encoded by an endogenous gene. Suitable methods for producing human-antibody transgenic animals are well known in the art. (See, for example, U.S. Pat. Nos. 5,939,598 and 6,075,181 (Kucherlapati et al.), U.S. Pat. Nos. 5,569,825, 5,545,806, 5,625,126, 5,633,425, 5,661,016, and 5,789,650 (Lonberg et al.), Jakobovits et al., Proc. Natl. Acad. Sci. USA, 90: 2551-2555 (1993), Jakobovits et al., Nature, 362: 255-258 (1993), Jakobovits et al. WO 98/50433, Jakobovits et al. WO 98/24893, Lonberg et al. WO 98/24884, Lonberg et al. WO 97/13852, Lonberg et al. WO 94/25585, Lonberg et al. EP 0 814 259 A2, Lonberg et al. GB 2 272 440 A, Lonberg et al., Nature 368:856-859 (1994), Lonberg et al., Int Rev Immunol 13(1):65-93 (1995), Kucherlapati et al. WO 96/34096, Kucherlapati et al. EP 0 463 151 B1, Kucherlapati et al. EP 0 710 719 A1, Surani et al. U.S. Pat. No. 5,545,807, Bruggemann et al. WO 90/04036, Bruggemann et al. EP 0 438 474 B1, Taylor et al., Int. Immunol. 6(4)579-591 (1994), Taylor et al., Nucleic Acids Research 20(23):6287-6295 (1992), Green et al., Nature Genetics 7:13-21 (1994), Mendez et al., Nature Genetics 15:146-156 (1997), Tuaillon et al., Proc Natl Acad Sci USA 90(8):3720-3724 (1993) and Fishwild et al., Nat Biotechnol 14(7):845-851 (1996), the teachings of each of the foregoing are incorporated herein by reference in their entirety.) Human-antibody transgenic animals can be immunized with a suitable antigen (e.g., human IL-1R1), and antibody producing cells can be isolated and fused to form hybridomas using conventional methods. Hybridomas that produce human antibodies having the desired characteristics (e.g., specificity, affinity) can be identified using any suitable assay (e.g., ELISA) and, if desired, selected and subcloned using suitable culture techniques.

Humanized antibodies and other CDR-grafted antibodies can be prepared using any suitable method. The CDRs of a CDR-grafted antibody can be derived from a suitable antibody which binds a serum albumin (referred to as a donor antibody). Other sources of suitable CDRs include natural and artificial serum albumin-specific antibodies obtained from human or nonhuman sources, such as rodent (e.g., mouse, rat, rabbit), chicken, pig, goat, non-human primate (e.g., monkey) or a library.

The framework regions of a humanized antibody are preferably of human origin, and can be derived from any human antibody variable region having sequence similarity to the analogous or equivalent region (e.g., heavy chain variable region or light chain variable region) of the antigen-binding region of the donor antibody. Other sources of framework regions of human origin include human variable region consensus sequences. (See, e.g., Kettleborough, C. A. et al., Protein Engineering 4:773-783 (1991); Carter et al., WO 94/04679; Kabat, E. A., et al., Sequences of Proteins of Immunological Interest, Fifth Edition, U.S. Department of Health and Human Services, U.S. Government Printing Office (1991)). Other types of CDR grafted antibodies can contain framework regions of suitable origin, such as framework regions encoded by germline antibody gene segments from horse, cow, dog, cat and the like.

Framework regions of human origin can include amino acid substitutions or replacements, such as “back mutations” which replace an amino acid residue in the framework region of human or animal origin with a residue from the corresponding position of the donor antibody. One or more mutations in the framework region can be made, including deletions, insertions and substitutions of one or more amino acids. Variants can be produced by a variety of suitable methods, including mutagenesis of nonhuman donor or acceptor human chains. (See, e.g., U.S. Pat. Nos. 5,693,762 (Queen et al.) and 5,859,205 (Adair et al.), the entire teachings of which are incorporated herein by reference.)

Constant regions of antibodies, antibody chains (e.g., heavy chain, light chain) or fragments or portions thereof, if present, can be derived from any suitable source. For example, constant regions of human, humanized and certain chimeric antibodies, antibody chains (e.g., heavy chain, light chain) or fragments or portions thereof, if present can be of human origin and can be derived from any suitable human antibody or antibody chain. For example, a constant region of human origin or portion thereof can be derived from a human κ or λ light chain, and/or a human γ (e.g., γ1, γ2, γ3, γ4), Ξ, α (e.g., α1, α2), δ or ε heavy chain, including allelic variants. In certain embodiments, the antibody or antigen-binding fragment (e.g., antibody of human origin, human antibody) can include amino acid substitutions or replacements that alter or tailor function (e.g., effector function). For example, a constant region of human origin (e.g., γ1 constant region, γ2 constant region) can be designed to reduce complement activation and/or Fc receptor binding. (See, for example, U.S. Pat. Nos. 5,648,260 (Winter et al.), 5,624,821 (Winter et al.) and 5,834,597 (Tso et al.), the entire teachings of which are incorporated herein by reference.) Preferably, the amino acid sequence of a constant region of human origin that contains such amino acid substitutions or replacements is at least about 95% identical over the full length to the amino acid sequence of the unaltered constant region of human origin, more preferably at least about 99% identical over the full length to the amino acid sequence of the unaltered constant region of human origin.

Humanized antibodies, CDR grafted antibodies or antigen-binding fragments of a humanized or CDR grafted antibody can be prepared using any suitable method. Several such methods are well-known in the art. (See, e.g., U.S. Pat. No. 5,225,539 (Winter), U.S. Pat. No. 5,530,101 (Queen et al.).) The portions of a humanized or CDR grafted antibody (e.g., CDRs, framework, constant region) can be obtained or derived directly from suitable antibodies (e.g., by de novo synthesis of a portion), or nucleic acids encoding an antibody or chain thereof having the desired property (e.g., binds serum albumin) can be produced and expressed. To prepare a portion of a chain, one or more stop codons can be introduced at the desired position. For example, nucleic acid (e.g., DNA) sequences coding for humanized or CDR grafted variable regions can be constructed using PCR mutagenesis methods to alter existing DNA sequences. (See, e.g., Kamman, M., et al., Nucl. Acids Res. 17:5404 (1989).) PCR primers coding for the new CDRs can be hybridized to a DNA template of a previously humanized variable region which is based on the same, or a very similar, human variable region (Sato, K., et al., Cancer Research 53:851-856 (1993)). If a similar DNA sequence is not available for use as a template, a nucleic acid comprising a sequence encoding a variable region sequence can be constructed from synthetic oligonucleotides (see e.g., Kolbinger, F., Protein Engineering 8:971-980 (1993)). A sequence encoding a signal peptide can also be incorporated into the nucleic acid (e.g., on synthesis, upon insertion into a vector). The natural signal peptide sequence from the acceptor antibody, a signal peptide sequence from another antibody or other suitable sequence can be used (see, e.g., Kettleborough, C. A., Protein Engineering 4:773-783 (1991)). Using these methods or other suitable methods, variants can be readily produced. In one embodiment, cloned variable regions can be mutated, and sequences encoding variants with the desired specificity can be selected (e.g., from a phage library; see, e.g., U.S. Pat. No. 5,514,548 (Krebber et al.) and WO 93/06213 (Hoogenboom et al.)).

The antibody or antigen-binding fragment that binds IL-1R1 can be a chimeric antibody or an antigen-binding fragment of a chimeric antibody. The chimeric antibody or antigen-binding fragment thereof comprises a variable region from one species (e.g., mouse) and at least a portion of a constant region from another species (e.g., human). Chimeric antibodies and antigen-binding fragments of chimeric antibodies can be prepared using any suitable method. Several suitable methods are well-known in the art. (See, e.g., U.S. Pat. No. 4,816,567 (Cabilly et al.), U.S. Pat. No. 5,116,946 (Capon et al.).)

A preferred method for obtaining antigen-binding fragments of antibodies that bind IL-1R1 comprises selecting an antigen-binding fragment (e.g., scFvs, dAbs) that has binding specificity for a desired IL-1R1 from a repertoire of antigen-binding fragments. For example, dAbs that bind IL-1R1 can be selected from a suitable phage display library. A number of suitable bacteriophage display libraries and selection methods (e.g., monovalent display and multivalent display systems) have been described. (See, e.g., Griffiths et al., U.S. Pat. No. 6,555,313 B1 (incorporated herein by reference); Johnson et al., U.S. Pat. No. 5,733,743 (incorporated herein by reference); McCafferty et al., U.S. Pat. No. 5,969,108 (incorporated herein by reference); Mulligan-Kehoe, U.S. Pat. No. 5,702,892 (incorporated herein by reference); Winter, G. et al., Annu. Rev. Immunol. 12:433-455 (1994); Soumillion, P. et al., Appl. Biochem. Biotechnol. 47(2-3):175-189 (1994); Castagnoli, L. et al., Comb. Chem. High Throughput Screen, 4(2):121-133 (2001); WO 99/20749 (Tomlinson and Winter); WO 03/002609 A2 (Winter et al.); WO 2004/003019A2 (Winter et al.).) The polypeptides displayed in a bacteriophage library can be displayed on any suitable bacteriophage, such as a filamentous phage (e.g., fd, M13, F1), a lytic phage (e.g., T4, T7, lambda), or an RNA phage (e.g., MS2), for example, and selected for binding to IL-1R1 (e.g., human IL-1R1).

Generally, a library of phage that displays a repertoire of polypeptides as fusion proteins with a suitable phage coat protein is used. Such a library can be produced using any suitable methods, such as introducing a library of phage vectors or phagemid vectors encoding the displayed antibodies or antigen-binding fragments thereof into suitable host bacteria, and culturing the resulting bacteria to produce phage (e.g., using a suitable helper phage or complementing plasmid if desired). The library of phage can be recovered from such a culture using any suitable method, such as precipitation and centrifugation.

The library can comprise a repertoire of antibodies or antigen-binding fragments thereof that contains any desired amount of amino acid sequence diversity. For example, the repertoire can contain antibodies or antigen-binding fragments thereof that have amino acid sequences that correspond to naturally occurring antibodies from a desired organism, and/or can contain one or more regions of random or randomized amino acid sequences (e.g., CDR sequences). The antibodies or antigen-binding fragments thereof in such a repertoire or library can comprise defined regions of random or randomized amino acid sequence and regions of common amino acid sequence. In certain embodiments, all or substantially all polypeptides in a repertoire are a desired type of antigen-binding fragment of an antibody (e.g., human V_{H} or human V_{L}). For example, each polypeptide in the repertoire can contain a V_{H}, a V_{L} or an Fv (e.g., a single chain Fv).

Amino acid sequence diversity can be introduced into any desired region of antibodies or antigen-binding fragments thereof using any suitable method. For example, amino acid sequence diversity can be introduced into a target region, such as a complementarity determining region of an antibody variable domain, by preparing a library of nucleic acids that encode the diversified antibodies or antigen-binding fragments thereof using any suitable mutagenesis methods (e.g., low fidelity PCR, oligonucleotide-mediated or site directed mutagenesis, diversification using NNK codons) or any other suitable method. If desired, a region of the antibodies or antigen-binding fragments thereof to be diversified can be randomized.

A suitable phage display library can be used to select antibodies or antigen-binding fragments of antibodies that bind IL-1R1, inhibit IL-1R1 function and have other beneficial properties. For example, antibodies or antigen-binding fragments that resist aggregation when unfolded can be selected. Aggregation is influenced by polypeptide concentration and is thought to arise in many cases from partially folded or unfolded intermediates. Factors and conditions that favor partially folded intermediates, such as elevated temperature and high polypeptide concentration, promote irreversible aggregation. (Fink, A. L., Folding & Design 3:R1-R23 (1998).)

For example, storing purified polypeptides in concentrated form, such as a lyophilized preparation, frequently results in irreversible aggregation of at least a portion of the polypeptides. Also, production of a polypeptide by expression in biological systems, such as E. coli, often results in the formation of inclusion bodies which contain aggregated polypeptides. Recovering active polypeptides from inclusion bodies can be very difficult and require adding additional steps, such as a refolding step, to a biological production system.

Antibodies and antigen-binding fragments that resist aggregation and unfold reversibly when heated can be selected from a suitable phage display library. Generally, a phage display library comprising a repertoire of displayed antibodies or antigen-binding fragments thereof is heated to a temperature (Ts) at which at least a portion of the displayed antibodies or antigen-binding fragments thereof are unfolded, then cooled to a temperature (Tc) wherein Ts>Tc, whereby at least a portion of the antibodies or antigen-binding fragments thereof have refolded and a portion of the polypeptides have aggregated. Then, antibodies or antigen-binding fragments thereof that unfold reversibly and bind serum albumin are recovered at a temperature (Tr). The recovered antibody or antigen-binding fragment thereof that unfolds reversibly has a melting temperature (Tm), and preferably, the repertoire was heated to Ts, cooled to Tc and the antibody or antigen-binding fragment thereof that unfolds reversibly was isolated at Tr, such that Ts>Tm>Tc, and Ts>Tm>Tr. Generally, the phage display library is heated to about 80° C. and cooled to about room temperature or about 4° C. before selection. Antibodies or antigen-binding fragment thereof that unfold reversibly and resist aggregation can also be designed or engineered by replacing certain amino acid residue with residues that confer the ability to unfold reversibly. (See, WO 2004/101790 (Jespers et al.), and U.S. Provisional Patent Application Nos. 60/470,340 (filed on May 14, 2003) and 60/554,021 (filed on Mar. 17, 2004) for detailed discussion of methods for selecting and for designing or engineering antibodies or antigen-binding fragments thereof that unfold reversibly. The teachings of WO 2004/101790 and both of the foregoing U.S. Provisional Patent Applications are incorporated herein by reference.)

Antibodies or antigen-binding fragments thereof that unfold reversibly and resist aggregation provide several advantages. For example, due to their resistance to aggregation, antibodies or antigen-binding fragments thereof that unfold reversibly can readily be produced in high yield as soluble proteins by expression using a suitable biological production system, such as E. coli. In addition, antibodies or antigen-binding fragments thereof that unfold reversibly can be formulated and/or stored at higher concentrations than conventional polypeptides, and with less aggregation and loss of activity. For example, dAb HEL4 is a human V_{H} that binds hen egg lysozyme and unfolds reversibly, and DOM7h-26 (SEQ ID NO: 743) is a human V_{H} that binds serum albumin and unfolds reversibly.

Preferably, the antibody or antigen-binding fragment thereof that binds IL-1R1 comprises a variable domain (V_{H}, V_{κ}, V_{λ}) in which one or more of the framework regions (FR) comprise (a) the amino acid sequence of a human framework region, (b) at least 8 contiguous amino acids of the amino acid sequence of a human framework region, or (c) an amino acid sequence encoded by a human germline antibody gene segment, wherein said framework regions are as defined by Kabat. In certain embodiments, the amino acid sequence of one or more of the framework regions is the same as the amino acid sequence of a corresponding framework region encoded by a human germline antibody gene segment, or the amino acid sequences of one or more of said framework regions collectively comprise up to 5 amino acid differences relative to the amino acid sequence of said corresponding framework region encoded by a human germline antibody gene segment.

In other embodiments, the amino acid sequences of FR1, FR2, FR3 and FR4 are the same as the amino acid sequences of corresponding framework regions encoded by a human germline antibody gene segment, or the amino acid sequences of FR1, FR2, FR3 and FR4 collectively contain up to 10 amino acid differences relative to the amino acid sequences of corresponding framework regions encoded by said human germline antibody gene segments. In other embodiments, the amino acid sequence of said FR1, FR2 and FR3 are the same as the amino acid sequences of corresponding framework regions encoded by said human germline antibody gene segment.

In particular embodiments, the antibody or antigen binding fragment that binds IL-1R1 comprises an immunoglobulin variable domain (e.g., V_{H}, V_{L}) based on a human germline sequence, and if desired can have one or more diversified regions, such as the complementarity determining regions. Suitable human germline sequence for V_{H} include, for example, sequences encoded by the V_{H} gene segments DP4, DP7, DP8, DP9, DP10, DP31, DP33, DP45, DP46, DP47, DP49, DP50, DP51, DP53, DP54, DP65, DP66, DP67, DP68 and DP69, and the J_{H} segments JH1, JH2, JH3, JH4, JH4b, JH5 and JH6. Any of the foregoing V_{H} gene segments can be paired with any of the foregoing J_{H} segments. Suitable human germline sequence for V_{L} include, for example, sequences encoded by the V_{κ} gene segments DPK1, DPK2, DPK3, DPK4, DPK5, DPK6, DPK7, DPK8, DPK9, DPK10, DPK12, DPK13, DPK15, DPK16, DPK18, DPK19, DPK20, DPK21, DPK22, DPK23, DPK24, DPK25, DPK26 and DPK28, and the J_{κ} segments J_{κ} 1, J_{κ} 2, J_{κ} 3, J_{κ} 4 and J_{κ} 5. Any of the foregoing V_{L} gene segments can be paired with any of the foregoing J_{κ} segments.

The antibody or antigen-binding fragment that bind IL-1R1 can bind IL-1R1 with any desired affinity, and antibodies and antigen-binding fragments with a desired affinity can be readily identified using any suitable screening method. The antibody or antigen-binding fragment that binds IL-1R1 (e.g., dAb) generally binds with a KD (KD=K_{off} (kd)/Kₒₙ (ka)) of about KD of 300 nM to 5 pM (ie, 3×10⁻⁷ to 5×10⁻¹² M), preferably 50 nM to 20 pM, more preferably 5 nM to 200 pM and most preferably 1 nM to 100 pM, for example 1×10⁻⁷ M or less, preferably 1×10⁻⁸ M or less, more preferably 1×10⁻⁹ M or less, advantageously 1×10⁻¹⁰ M or less and most preferably 1×10⁻¹¹ M or less; and/or a K_{off} rate constant of 5×10⁻¹ s⁻¹ to 1×10⁻⁷ s⁻¹ preferably 1×10⁻² s⁻¹ to 1×10⁻⁶ s⁻¹, more preferably 5×10⁻³ s⁻¹ to 1×10⁻⁵ s⁻¹, for example 5×10⁻¹ s⁻¹ or less, preferably 1×10⁻² s⁻¹ or less, advantageously 1×10⁻³ s⁻¹ or less, more preferably 1×10⁻⁴ s⁻¹ or less, still more preferably 1×10⁻⁵ s⁻¹ or less, and most preferably 1×10⁻⁶ s⁻¹ or less as determined by surface plasmon resonance. Certain antibody or antigen-binding fragment that bind IL-1R1, specifically bind human IL-1R1 with a KD of 50 nM to 20 pM, and a K_{off} rate constant of 5×10⁻¹ s⁻¹ to 1×10⁻⁷ s⁻¹, as determined by surface plasmon resonance.

Preferably, the antibody or antigen-binding fragment that bind IL-1R1 inhibits binding of IL-1a and/or IL-1β to IL-1R1 with an inhibitory concentration 50 (IC50) that is ≦10 μM, ≦1 μM, ≦100 nM, ≦10 nM, ≦1 nM, ≦500 pM, ≦300 pM, ≦100 pM, or ≦10 pM. The IC50 is preferably determined using an in vitro receptor binding assay, such as the assay described herein.

It is also preferred that the antibody or antigen-binding fragment that bind IL-1R1 inhibits IL-1\ddot{□} and/or IL-1□-induced functions in a suitable in vitro assay with a neutralizing dose 50 (ND50) that is ≦10 □M, ≦1 □M, ≦100 nM, ≦10 nM, 1 nM, ≦500 pM, ≦300 pM, ≦100 pM, or ≦10 pM. For example, the antibody or antigen-binding fragment that bind IL-1R1 can inhibit IL-1□- or IL-1□-induced release of Interleukin-8 by MRC-5 cells (ATCC Accession No. CCL-171) in an in vitro assay, such as the assay described herein. In another example, the antibody or antigen-binding fragment that bind IL-1R1 can inhibit IL-1□- or IL-1□-induced release of Interleukin-6 in a whole blood assay, such as the assay described herein.

In particular embodiments, the antagonist of IL-1R1 comprises an antagonist of IL-1R1 moiety that is a dAb. For example, the antagonist of IL-1R1 comprises a dAb that competes with a dAb for binding to IL-1R1, wherein the dAb is selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-12-2-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO: 94), DOM4-129-23 (SEQ ID NO: 195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ TD NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

In other embodiments, the antagonists of IL-1R1 comprises a dAb having an amino acid sequence that has at least about 80%, or at least about 85%, or at least about 90%, or at least about 95%, or at least about 96%, or at least about 97%, or at least about 98%, or at least about 99% amino acid sequence identity with DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-1130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

Amino acid sequence identity is preferably determined using a suitable sequence alignment algorithm and default parameters, such as BLAST P (Karlin and Altschul, Proc. Natl. Acad. Sci. USA 87(6):2264-2268 (1990)).

In other embodiments, the antagonists of IL-1R1 comprises a e that has an amino acid sequence selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:1100), DOM4-122-6 (SEQ ID NO:1101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

In other embodiments, the antagonist of IL-1R1 comprises a dAb that has binding specificity for IL-1R1 and comprises the CDRs of any of the foregoing amino acid sequences.

Antibodies and Antibody Portions that Bind a Polypeptide that Enhances Serum Half-Life

As described herein, in some embodiments, the antagonist of IL-1R1 comprises a moiety that binds a polypeptide that enhances serum half-life (e.g., serum albumin, neonatal Fc receptor). The antibody or antigen-binding fragment that has binding specificity for polypeptide that enhances serum half-life generally has binding specificity for a polypeptide form an animal to which the antagonist of IL-1R1 will be administered. Preferably, the antibody or antigen-binding fragment has binding specificity for human serum albumin or human neonatal Fc receptor. However, veterinary applications are contemplated and the antibody or antigen-binding fragment can have binding specificity for a polypeptide that enhances serum half-life from a desired animal, for example serum albumin from dog, cat, horse, cow, chicken, sheep, pig, goat, deer, mink, and the like. In some embodiments the antibody or antigen-binding fragment has binding specificity for a polypeptide that enhances serum half-life from more than one species. Such antibodies or antigen-binding fragment provide the advantage of allowing preclinical and clinical studies to be designed and executed using the same antagonist of IL-1R1, and obviate the need to conduct preclinical studies with a suitable surrogate antagonist of IL-1R1.

Suitable antibodies and antigen-binding fragments of antibodies that bind a polypeptide that enhances serum half-life can have the features and properties described in detail herein with respect to antibodies and antigen-binding portions thereof that bind IL-1R1, and can be prepared using any suitable method. For example, antibodies and antigen-binding fragments thereof that bind a polypeptide that enhances serum half-life (e.g., serum albumin, neonatal Fc receptor) be prepared by immunization and/or screening using a selected a polypeptide that enhances serum half-life (e.g., serum albumin, neonatal Fc receptor).

In certain embodiments, the antagonist of IL-1R1 does not contain a mouse, rat and/or rabbit antibody that binds serum albumin or antigen-binding fragment of such an antibody.

The antibody or antigen-binding fragment can bind serum albumin with any desired affinity, on rate and off rate. The affinity (KD), on rate (Kₒₙ or kₐ) and off rate (K_{off} or k_{d}) can be selected to obtain a desired serum half-life for a particular drug. For example, it may be desirable to obtain a maximal serum half-life for treating a chronic inflammation or a chronic inflammatory disorder, while a shorter half-life may be desirable for a diagnostic applications or for treating acute inflammation or an acute disorder. Generally, a fast on rate and a fast or moderate off rate for binding to serum albumin is preferred. Antagonists of IL-1R1 that comprise an antibody or antigen-binding fragment thereof that binds serum albumin with these characteristics will quickly bind serum albumin after being administered, and will dissociate and rebind serum albumin rapidly. These characteristics will reduce rapid clearance of the antagonist of IL-1R1 (e.g., through the kidneys) but still provide efficient delivery and access to the drug target.

The antigen-binding fragment that binds serum albumin (e.g., dAb) generally binds with a KD of about 1 nM to about 500 μM. In some embodiments, the antigen-binding fragment binds serum albumin with a KD (KD=K_{off} (kd)/Kₒₙ (ka)) of about 10 to about 100 nM, or about 100 nM to about 500 nM, or about 500 nM to about 5 mM, as determined by surface plasmon resonance (e.g., using a BIACORE instrument). In particular embodiments, the drug conjugate, noncovalent drug conjugate or drug fusion comprises and antigen-binding fragment of an antibody (e.g., a dAb) that binds serum albumin (e.g., human serum albumin) with a KD of about 50 nM, or about 70 nM, or about 100 nM, or about 150 nM or about 200 mM. The improved pharmacokinetic properties (e.g., prolonged t1/2β, increased AUC) of drug conjugates, noncovalent drug conjugates and drug fusions described herein may correlate with the affinity of the antigen-binding fragment that binds serum albumin. Accordingly, drug conjugates, noncovalent drug conjugates and drug fusions that have improved pharmacokinetic properties can generally be prepared using an antigen-binding fragment that binds serum albumin (e.g., human serum albumin) with high affinity (e.g., KD of about 500 nM or less, about 250 nM or less, about 100 nM or less, about 50 nM or less, about 10 nM or less, or about 1 nM or less, or about 100 pM or less).

Preferably, the drug that is conjugated or fused to the antigen-binding fragment that binds serum albumin, binds to its target (the drug target) with an affinity (KD) that is stronger than the affinity of the antigen-binding fragment for serum albumin and/or a K_{off} (kd) that is faster that the K_{off} of the antigen binding fragment for serum albumin, as measured by surface plasmon resonance (e.g., using a BIACORE instrument). For example, the drug can bind its target with an affinity that is about 1 to about 100000, or about 100 to about 100000, or about 1000 to about 100000, or about 10000 to about 100000 times stronger than the affinity of antigen-binding fragment that binds SA for SA. For example, the antigen-binding fragment of the antibody that binds SA can bind with an affinity of about 10 μM, while the drug binds its target with an affinity of about 100 pM.

In particular embodiments, the antigen-binding fragment of an antibody that binds serum albumin is a dAb that binds human serum albumin. For example, a V_{κ} dAb having an amino acid sequence selected from the group consisting of DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-8 (SEQ ID NO:746), DOM7r-13 (SEQ ID NO:747), and DOM7r-14 (SEQ ID NO:748), or a V_{H} dAb having an amino acid sequence selected from the group consisting of DOM7h-22 (DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), and DOM7h-27 (SEQ ID NO:745). In other embodiments, the antigen-binding fragment of an antibody that binds serum albumin is a dAb that binds human serum albumin and comprises the CDRs of any of the foregoing amino acid sequences.

In other embodiments, the antigen-binding fragment of an antibody that binds serum albumin is a dAb that binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7h-8 (SEQ ID NO:746), DOM7r-13 (SEQ ID NO:747), DOM7r-14 (SEQ ID NO:748), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), DOM7r-33 (SEQ ID NO:767), Sequence A (SEQ ID NO:768), Sequence B (SEQ ID NO:769), Sequence C (SEQ ID NO:770), Sequence D (SEQ ID NO:771), Sequence E (SEQ ID NO:772), Sequence F (SEQ ID NO:773), Sequence G (SEQ ID NO:774), Sequence H (SEQ ID NO:775), Sequence I (SEQ ID NO:776), Sequence J (SEQ ID NO:777), Sequence K (SEQ ID NO:778), Sequence L (SEQ ID NO:779), Sequence M (SEQ ID NO:780), Sequence N (SEQ ID NO:781), Sequence O (SEQ ID NO:782), Sequence P (SEQ ID NO:783), and Sequence Q (SEQ ID NO:784).

Amino acid sequence identity is preferably determined using a suitable sequence alignment algorithm and default parameters, such as BLAST P (Karlin and Altschul, Proc. Natl. Acad. Sci. USA 87(6):2264-2268 (1990)).

### Antagonist of IL-1R1 Formats

Antagonist of IL-1R1 moieties (e.g., IL-1ra or a functional variant thereof, dAb) can be formatted into a variety of suitable structures for use in the invention. For example, as described in detail herein, an antagonist of IL-1R1 moiety (e.g., a dAb that binds IL-1R1 and inhibits a function of IL-1R1) can be formatted as a conjugate and protein, polypeptide, and peptide antagonists of IL-1R1 moieties can be formatted as a fusion protein. A protein, polypeptide or peptide antagonist of IL-1R1 (e.g., a dAb that binds IL-1R1 and inhibits a function of IL-1R1) can be formatted as a mono or multispecific antibody or antibody fragment, or into a mono or multispecific non-antibody structure. Suitable formats include, any suitable polypeptide structure in which IL-1ra, a functional variant of IL-1ra, an antibody variable domain or one or more of the CDRs thereof can be incorporated, so as to confer binding specificity for IL-1R1 on the structure. A variety of suitable antibody formats are known in the art, such as, IgG-like formats, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a single variable domain (e.g., V_{H}, V_{L}, V_{HH}), a dAb, and modified versions of any of the foregoing (e.g., modified by the covalent attachment of polyalkylene glycol (e.g., polyethylene glycol, polypropylene glycol, polybutylene glycol) or other suitable polymer). See, PCT/GB03/002804, filed Jun. 30, 2003, which designated the United States, (WO 2004/081026) regarding PEGylated single variable domains and dAbs, suitable methods for preparing same, increased in vivo half life of the PEGylated single variable domains and dAb monomers and multimers, suitable PEGs, preferred hydrodynamic sizes of PEGs, and preferred hydrodynamic sizes of PEGylated single variable domains and dAb monomers and multimers. The entire teaching of PCT/GB03/002804 (WO 2004/081026), including the portions referred to above, are incorporated herein by reference.

The antagonist of IL-1R1 can be formatted as a dimer, trimer or polymer of a desired dAb monomer that binds IL-1R1, for example, using a suitable linker such as (Gly₄Ser)ₙ, where n=from 1 to 8, e.g., 1, 2, 3, 4, 5, 6, 7 or 8. If desired, a protein, polypeptide or peptide antagonist of IL-1R1 moiety (including dAb monomers, dimers and trimers, IL-1ra and functional variants thereof) can be linked to an antibody Fc region. For example, a protein, polypeptide or peptide antagonist can be linked to a human IgG (Fc region) comprising one or both of C_{H}2 and C_{H}3 domains, and optionally a hinge region, and optionally containing mutations that reduce the ability of the Fc region to fix complement and/or bind Fc receptors. Such mutations are well-known in the art and described, for example, in GB 2,209,757 B (Winter et al.), WO 89/07142 (Morrison et al.), and WO 94/29351 (Morgan et al.), the teachings of these documents with respect to amino acid mutations in Fc regions that reduce Fc receptor binding and/or the ability to fix complement are incorporated herein by reference.

Protein, polypeptide or peptide antagonists of IL-1R1 moieties (e.g., dAb monomers, IL-1ra or functional variants thereof) can also be combined and/or formatted into non-antibody multivalent complexes that comprise two or more copies of the same antagonist of IL-1R1 moiety or two or more different antagonist of IL-1R1 moieties, and which bind cells expressing IL-1R1 with superior avidity. For example natural bacterial receptors such as SpA can been used as scaffolds for the grafting of CDRs to generate non-antibody formats that bind specifically to one or more epitopes of IL-1R1. Details of this procedure are described in U.S. Pat. No. 5,831,012. Other suitable scaffolds include those based on fibronectin and affibodies. Details of suitable procedures are described in WO 98/58965. Other suitable scaffolds include lipocallin and CTLA4, as described in van den Beuken et al., J. Mol. Biol. 310:591-601 (2001), and scaffolds such as those described in WO 00/69907 (Medical Research Council), which are based for example on the ring structure of bacterial GroEL or other chaperone polypeptides. Protein scaffolds may be combined; for example, CDRs may be grafted on to a CTLA4 scaffold and used together with immunoglobulin V_{H} or V_{L} domains to form an antagonist of IL-1R1 suitable for use in the invention. Likewise, fibronectin, lipocallin and other scaffolds may be combined

A variety of suitable methods for preparing any desired format are known in the art. For example, antibody chains and formats (e.g., IgG-like formats, chimeric antibodies, humanized antibodies, human antibodies, single chain antibodies, bispecific antibodies, antibody heavy chains, antibody light chains, homodimers and heterodimers of antibody heavy chains and/or light chains) can be prepared by expression of suitable expression constructs and/or culture of suitable cells (e.g., hybridomas, heterohybridomas, recombinant host cells containing recombinant constructs encoding the format). Further, formats such as antigen-binding fragments of antibodies or antibody chains (e.g., a Fv fragment (e.g., single chain Fv (scFv), a disulfide bonded Fv), a Fab fragment, a Fab′ fragment, a F(ab′)₂ fragment), can be prepared by expression of suitable expression constructs or by enzymatic digestion of antibodies, for example using papain or pepsin.

A protein, polypeptide or peptide antagonist of IL-1R1 moiety can be formatted as a “dual specific ligand” or a “multispecific ligand,” as described in WO 03/002609, the entire teachings of which are incorporated herein by reference. Dual specific ligand comprises immunoglobulin single variable domains that have different binding specificities. Such dual specific ligands can comprise combinations of heavy and light chain domains. For example, the dual specific ligand may comprise a V_{H} domain and a V_{L} domain, which may be linked together in the form of an scFv (e.g., using a suitable linker such as Gly₄Ser), or formatted into a bispecific antibody or antigen-binding fragment thereof (e.g. F(ab′)₂ fragment). The dual specific ligands do not comprise complementary V_{H}/V_{L} pairs which form a conventional two chain antibody antigen-binding site that binds antigen or epitope co-operatively. Instead, the dual format ligands comprise a V_{H}/V_{L} complementary pair, wherein the V domains have different binding specificities. A dual specific ligand can comprise one or more C_{H} or C_{L} domains if desired. A hinge region domain may also be included if desired. Such combinations of domains may, for example, mimic natural antibodies, such as IgG or IgM, or fragments thereof, such as Fv, scFv, Fab or F(ab′)₂ molecules. Other structures, such as a single arm of an IgG molecule comprising V_{H}, V_{L}, C_{H}1 and C_{L} domains, are envisaged. Preferably, the dual specific ligand comprises only two variable domains although several such ligands may be incorporated together into the same protein, for example two such ligands can be incorporated into an IgG or a multimeric immunoglobulin, such as IgM. Alternatively, a plurality of dual specific ligands can be combined to form a multimer. For example, two different dual specific ligands can be combined to create a tetra-specific molecule. It will be appreciated by one skilled in the art that the light and heavy variable regions of a dual-specific ligand can be on the same polypeptide chain, or alternatively, on different polypeptide chains. In the case that the variable regions are on different polypeptide chains, then they may be linked via a linker, generally a flexible linker (such as a polypeptide chain), a chemical linking group, or any other method known in the art.

A multispecific ligand possess more than one epitope binding specificity. Generally, the multi-specific ligand comprises two or more epitope binding domains, such as dAbs or non-antibody protein domain comprising a binding site for an epitope, e.g., an affibody, an SpA domain, an LDL receptor class A domain, an EGF domain, an avimer. Multispecific ligands can be formatted further as described herein.

In some embodiments, the antagonist of IL-1R1 is an IgG-like format. Such formats have the conventional four chain structure of an IgG molecule (2 heavy chains and two light chains), in which one or more of the variable regions (V_{H} and or V_{L}) have been replaced with a dAb or single variable domain that has binding specificity for IL-1R1. Preferably, each of the variable regions (2 V_{H} regions and 2 V_{L} regions) is replaced with a dAb or single variable domain. The dAb(s) or single variable domain(s) that are included in an IgG-like format can have the same specificity or different specificities. In some embodiments, the IgG-like format is tetravalent and can have one, two, three or four specificities. For example, the IgG-like format can be monospecific and comprises 4 dAbs that have the same specificity (e.g., for the same epitope on IL-1R1); bispecific and comprises 3 dAbs that have the same specificity and another dAb that has a different specificity; bispecific and comprise two dAbs that have the same specificity and two dAbs that have a common but different specificity; trispecific and comprises first and second dAbs that have the same specificity, a third dAbs with a different specificity and a fourth dAb with a different specificity from the first, second and third dAbs; or tetraspecific and comprise four dAbs that each have a different specificity. Antigen-binding fragments of IgG-like formats (e.g., Fab, F(ab′)₂, Fab′, Fv, scFv) can be prepared. Preferably, the IgG-like formats or antigen-binding fragments thereof do not crosslink IL-1R1.

### Half-Life Extended Formats

An antagonist of IL-1R1 or antagonist of IL-1R1 moiety (e.g., dAb monomer, dimer or multimer, dual specific format, multi-specific format) can be formatted to extend its in vivo serum half life. Increased in vivo half-life is useful in in vivo applications of polypeptides, such as immunoglobulins, especially antibodies and most especially antibody fragments of small size such as dAbs. Such fragments (Fvs, disulphide bonded Fvs, Fabs, scFvs, dAbs) are rapidly cleared from the body, which can severely limit clinical applications.

An antagonist of IL-1R1 or antagonist of IL-1R1 moiety can be formatted to have a larger hydrodynamic size, for example, by attachment of a polyalkyleneglycol group (e.g. polyethyleneglycol (PEG) group), serum albumin, transferrin, transferrin receptor or at least the transferrin-binding portion thereof, an antibody Fc region, or by conjugation to an antibody domain. In some embodiments, the antagonist if IL-1R1 (e.g., ligand, dAb monomer) is PEGylated. Preferably the PEGylated antagonist IL-1R1 (e.g., ligand, dAb monomer) binds IL-1R1 with substantially the same affinity as the same antagonist that is not PEGylated. For example, the antagonist of IL-1R1 can be a PEGylated dAb monomer that binds IL-1R1, wherein the PEGylated dAb monomer binds IL-1R1 with an affinity that differs from the affinity of dAb in unPEGylated form by no more than a factor of about 1000, preferably no more than a factor of about 100, more preferably no more than a factor of about 10, or with affinity substantially unchanged affinity relative to the unPEGylated form.

Examples of suitable albumin, albumin fragments or albumin variants for use in an antagonists of IL-1R1 are described in WO 2005/077042A2, which is incorporated herein by reference in its entirety. In particular, the following albumin, albumin fragments or albumin variants can be used in the present invention:
SEQ ID NO:1 (as disclosed in WO 2005/077042A2, this sequence being explicitly incorporated into the present disclosure by reference);
Albumin fragment or variant comprising or consisting of amino acids 1-387 of SEQ ID NO:1 in WO 2005/077042A2;
Albumin, or fragment or variant thereof, comprising an amino acid sequence selected from the group consisting of: (a) amino acids 54 to 61 of SEQ ID NO:1 in WO 2005/077042A2; (b) amino acids 76 to 89 of SEQ ID NO:1 in WO 2005/077042A2; (c) amino acids 92 to 100 of SEQ ID NO:1 in WO 2005/077042A2; (d) amino acids 170 to 176 of SEQ ID NO:1 in WO 2005/077042A2; (e) amino acids 247 to 252 of SEQ ID NO:1 in WO 2005/077042A2; (f) amino acids 266 to 277 of SEQ ID NO:1 in WO 2005/077042A2; (g) amino acids 280 to 288 of SEQ ID NO:1 in WO 2005/077042A2; (h) amino acids 362 to 368 of SEQ ID NO:1 in WO 2005/077042A2; (i) amino acids 439 to 447 of SEQ ID NO:1 in WO 2005/077042A2; amino acids 462 to 475 of SEQ ID NO:1 in WO 2005/077042A2; (j) amino acids 478 to 486 of SEQ ID NO:1 in WO 2005/077042A2; and (l) amino acids 560 to 566 of SEQ ID NO: 1 in WO 2005/077042A2.

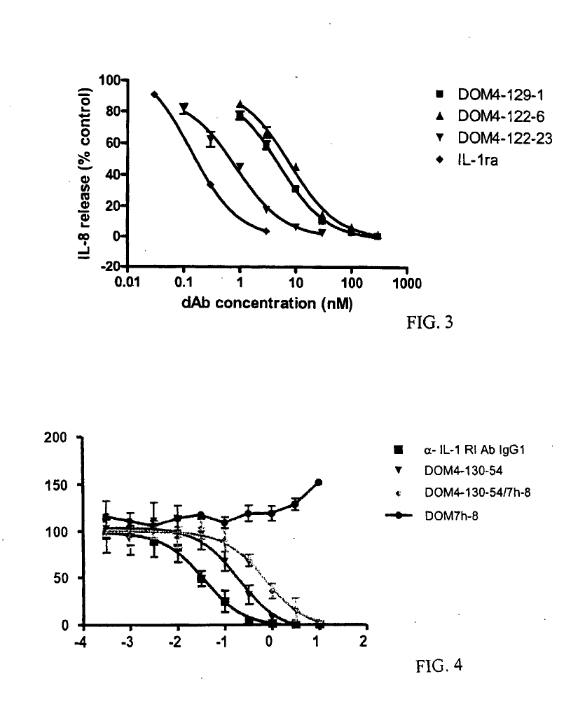

Further examples of suitable albumin, fragments and analogs for use in an antagonist of IL-1R1 according to the invention are described in WO 03/076567A2, which is incorporated herein by reference in its entirety. In particular, the following albumin, fragments or variants can be used in the present invention:
Human serum albumin as described in WO 03/076567A2, eg, in FIG. 3 (this sequence information being explicitly incorporated into the present disclosure by reference);
Human serum albumin (HA) consisting of a single non-glycosylated polypeptide chain of 585 amino acids with a formula molecular weight of 66,500 (See, Meloun, et al., FEBS Letters 58:136 (1975); Behrens, et al., Fed. Proc. 34:591 (1975); Lawn, et al., Nucleic Acids Research 9:6102-6114 (1981); Minghetti, et al., J. Biol. Chem. 261:6747 (1986));
A polymorphic variant or analog or fragment of albumin as described in Weitkamp, et al., Ann. Hum. Genet. 37:219 (1973);
An albumin fragment or variant as described in EP 322094, eg, HA(1-373., HA(1-388), HA(1-389), HA(1-369), and HA(1-419) and fragments between 1-369 and 1-419;
An albumin fragment or variant as described in EP 399666, eg, HA(1-177) and HA(1-200) and fragments between HA(1-X), where X is any number from 178 to 199.

Where a (one or more) half-life extending moiety (e.g., albumin, transferrin and fragments and analogues thereof) is used in the antagonist of IL-1R1, it can be conjugated using any suitable method, such as, by direct fusion to an antagonist of IL-1R1 moiety, for example by using a single nucleotide construct that encodes a fusion protein, wherein the fusion protein is encoded as a single polypeptide chain with the half-life extending moiety located N- or C-terminally to the antagonist of IL-1R1 moiety. Alternatively, conjugation can be achieved by using a peptide linker between moieties, e.g., a peptide linker as described in WO 03/076567A2 or WO 2004/003019 (these linker disclosures being incorporated by reference in the present disclosure to provide examples for use in the present invention).

Small antagonists of IL-1R1 or antagonist of IL-1R1 moieties, such as a dAb monomer, can be formatted as a larger antigen-binding fragment of an antibody or as and antibody (e.g., formatted as a Fab, Fab′, F(ab)₂, F(ab′)₂, IgG, scFv). The hydrodynamic size of an antagonist of IL-1R1 (e.g., dAb monomer) and its serum half-life can also be increased by conjugating or linking the antagonist of IL-1R1 to a binding domain (e.g., antibody or antibody fragment) that binds an antigen or epitope that increases half-live in vivo, as described herein. For example, the antagonist of IL-1R1 (e.g., dAb monomer) can be conjugated or linked to an anti-serum albumin or anti-neonatal Fc receptor antibody or antibody fragment, e.g. an anti-SA or anti-neonatal Fc receptor dAb, Fab, Fab′ or scFv, or to an anti-SA affibody or anti-neonatal Fc receptor affibody.

Typically, a polypeptide that enhances serum half-life in vivo is a polypeptide which occurs naturally in vivo and which resists degradation or removal by endogenous mechanisms which remove unwanted material from the organism (e.g., human). For example, a polypeptide that enhances serum half-life in vivo can be selected from proteins from the extracellular matrix, proteins found in blood, proteins found at the blood brain barrier or in neural tissue, proteins localized to the kidney, liver, lung, heart, skin or bone, stress proteins, disease-specific proteins, or proteins involved in Fc transport.

Suitable polypeptides that enhance serum half-life in vivo include, for example, transferrin receptor specific ligand-neuropharmaceutical agent fusion proteins (see U.S. Pat. No. 5,977,307, the teachings of which are incorporated herein by reference), brain capillary endothelial cell receptor, transferrin, transferrin receptor (e.g., soluble transferrin receptor), insulin, insulin-like growth factor 1 (IGF 1) receptor, insulin-like growth factor 2 (IGF 2) receptor, insulin receptor, blood coagulation factor X, α1-antitrypsin and HNF 1α. Suitable polypeptides that enhance serum half-life also include alpha-1 glycoprotein (orosomucoid; AAG), alpha-1 antichymotrypsin (ACT), alpha-1 microglobulin (protein HC; AIM), antithrombin III (AT III), apolipoprotein A-1 (Apo A-1), apolipoprotein B (Apo B), ceruloplasmin (Cp), complement component C3 (C3), complement component C4 (C4), C1 esterase inhibitor (C1 INH), C-reactive protein (CRP), ferritin (FER), hemopexin (HPX), lipoprotein(a) (Lp(a)), mannose-binding protein (MBP), myoglobin (Myo), prealbumin (transthyretin; PAL), retinol-binding protein (RBP), and rheumatoid factor (RF).

Suitable proteins from the extracellular matrix include, for example, collagens, laminins, integrins and fibronectin. Collagens are the major proteins of the extracellular matrix. About 15 types of collagen molecules are currently known, found in different parts of the body, e.g. type I collagen (accounting for 90% of body collagen) found in bone, skin, tendon, ligaments, cornea, internal organs or type II collagen found in cartilage, vertebral disc, notochord, and vitreous humor of the eye.

Suitable proteins from the blood include, for example, plasma proteins (e.g., fibrin, α-2 macroglobulin, serum albumin, fibrinogen (e.g., fibrinogen A, fibrinogen B), serum amyloid protein A, haptoglobin, profilin, ubiquitin, uteroglobulin and β-2-microglobulin), enzymes and enzyme inhibitors (e.g., plasminogen, lysozyme, cystatin C, alpha-1-antitrypsin and pancreatic trypsin inhibitor), proteins of the immune system, such as immunoglobulin proteins (e.g., IgA, IgD, IgE, IgG, IgM, immunoglobulin light chains (kappa/lambda)), transport proteins (e.g., retinol binding protein, α-1 microglobulin), defensins (e.g., beta-defensin 1, neutrophil defensin 1, neutrophil defensin 2 and neutrophil defensin 3) and the like.

Suitable proteins found at the blood brain barrier or in neural tissue include, for example, melanocortin receptor, myelin, ascorbate transporter and the like.

Suitable polypeptides that enhances serum half-life in vivo also include proteins localized to the kidney (e.g., polycystin, type IV collagen, organic anion transporter K1, Heymann's antigen), proteins localized to the liver (e.g., alcohol dehydrogenase, G250), proteins localized to the lung (e.g., secretory component, which binds IgA), proteins localized to the heart (e.g., HSP 27, which is associated with dilated cardiomyopathy), proteins localized to the skin (e.g., keratin), bone specific proteins such as morphogenic proteins (BMPs), which are a subset of the transforming growth factor β superfamily of proteins that demonstrate osteogenic activity (e.g., BMP-2, BMP-4, BMP-5, BMP-6, BMP-7, BMP-8), tumor specific proteins (e.g., trophoblast antigen, herceptin receptor, oestrogen receptor, cathepsins (e.g., cathepsin B, which can be found in liver and spleen)).

Suitable disease-specific proteins include, for example, antigens expressed only on activated T-cells, including LAG-3 (lymphocyte activation gene), osteoprotegerin ligand (OPGL; see Nature 402, 304-309 (1999)), OX40 (a member of the TNF receptor family, expressed on activated T cells and specifically up-regulated in human T cell leukemia virus type-I (HTLV-I)-producing cells; see Immunol. 165 (1):263-70 (2000)). Suitable disease-specific proteins also include, for example, metalloproteases (associated with arthritis/cancers) including CG6512 Drosophila, human paraplegin, human FtsH, human AFG3L2, murine ftsH; and angiogenic growth factors, including acidic fibroblast growth factor (FGF-1), basic fibroblast growth factor (FGF-2), vascular endothelial growth factor/vascular permeability factor (VEGF/VPF), transforming growth factor-α (TGF α), tumor necrosis factor-alpha (TNF-α), angiogenin, interleukin-3 (IL-3), interleukin-8 (IL-8), platelet-derived endothelial growth factor (PD-ECGF), placental growth factor (P1GF), midkine platelet-derived growth factor-BB (PDGF), and fractalkine.

Suitable polypeptides that enhance serum half-life in vivo also include stress proteins such as heat shock proteins (HSPs). HSPs are normally found intracellularly. When they are found extracellularly, it is an indicator that a cell has died and spilled out its contents. This unprogrammed cell death (necrosis) occurs when as a result of trauma, disease or injury, extracellular HSPs trigger a response from the immune system. Binding to extracellular HSP can result in localizing the compositions of the invention to a disease site.

Suitable proteins involved in Fc transport include, for example, Brambell receptor (also known as FcRB). This Fc receptor has two functions, both of which are potentially useful for delivery. The functions are (1) transport of IgG from mother to child across the placenta (2) protection of IgG from degradation thereby prolonging its serum half-life. It is thought that the receptor recycles IgG from endosomes. (See, Holliger et al, Nat Biotechnol 15(7):632-6 (1997).)

Methods for pharmacokinetic analysis and determination of half-life will be familiar to those skilled in the art. Details may be found in Kenneth, A et al: Chemical Stability of Pharmaceuticals: A Handbook for Pharmacists and in Peters et al, Pharmacokinetc analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t alpha and t beta half lives and area under the curve (AUC).

Particular examples of half-life extended formats are described further below.

### Antagonist of IL-1R1 Fusion Proteins

Antagonist of IL-1R1 fusion proteins suitable for use in the invention are fusion proteins that comprise a continuous polypeptide chain, said chain comprising an antigen-binding fragment of an antibody that binds a polypeptide that extends serum half-life (e.g., serum albumin) as a first moiety, linked to a second moiety (antagonist of IL-1R1 moiety) that is a polypeptide antagonist of IL-1R1. The first and second moieties can be directly bonded to each other through a peptide bond, or linked through a suitable amino acid, or peptide or polypeptide linker. Additional moieties (e.g., third, fourth) and/or linker sequences can be present as appropriate. The first moiety can be in an N-terminal location, C-terminal location or internal relative to the second moiety (i.e., the polypeptide antagonist of IL-1R1). The moieties can occur on the continuous polypeptide chain in any desired order. In certain embodiments, each moiety can be present in more than one copy. For example, the antagonist of IL-1R1 fusion can comprise two or more first moieties each comprising an antigen-binding fragment of an antibody that binds a polypeptide that enhances serum half-life (e.g., a V_{H} that binds human serum albumin and a V_{L} that bind human serum albumin or two or more V_{H}s or V_{L}s that bind human serum albumin).

In certain embodiments, the fusion protein is a continuous polypeptide chain that has the formula (amino-terminal to carboxy-terminal):

a-(P)n2-b-(X)n1-c-(O)n3-d or a-(O)n3-b-(X)n1-c-(P)n2-d

wherein X is a polypeptide antagonist of IL-1R1 moiety;

P and Q are each independently a polypeptide binding moiety that contains a binding site that has binding specificity for a polypeptide that enhances serum half-life in vivo;

a, b, c and d are each independently absent or one to about 100 amino acid residues;

n1, n2 and n3 represent the number of X, P or Q moieties present, respectively;

n1 is one to about 10;

n2 is zero to about 10; and

n3 is zero to about 10, with the proviso that both n2 and n3 are not zero.

In some embodiments, when n1 and n2 are both one and n3 is zero, X does not comprise an antibody chain or a fragment of an antibody chain.

In some embodiments, n2 is one, two, three, four, five or six, and n3 is zero. In other embodiments, n3 is one, two, three, four, five or six, and n2 is zero. In other embodiments, n1, n2 and n3 are each one.

In certain embodiments, X does not comprises an antibody chain or a fragment of an antibody chain.

In preferred embodiments, P and Q are each independently a polypeptide binding moiety that has binding specificity for serum albumin.

In some embodiments, the antagonist of IL-1R1 fusion protein is a continuous polypeptide chain that has the formula:

a-(X)ₙ₁-b-(Y)ₙ₂-c-(Z)ₙ₃-d or a-(Z)ₙ₃-b-(Y)ₙ₂-c-(X)ₙ₁-d;

wherein X is a polypeptide that has binding specificity for IL-1R1;

Y is a single chain antigen-binding fragment of an antibody that has binding specificity for serum albumin;

Z is a polypeptide drug that has binding specificity for a second target;

a, b, c and d are each independently absent or one to about 100 amino acid residues;

n1 is one to about 10;

n2 is one to about 10; and

n3 is zero to about 10.

In some embodiments, when n1 and n2 are both one and n3 is zero, X does not comprise an antibody chain or a fragment of an antibody chain.

In one embodiment, neither X nor Z comprises an antibody chain or a fragment of an antibody chain. In one embodiment, nil is one, n3 is one and n2 is two, three, four, five, six, seven, eight or nine. Preferably, Y is an immunoglobulin heavy chain variable domain (V_{H}, V_{HH}) that has binding specificity for serum albumin, or an immunoglobulin light chain variable domain (V_{L}) that has binding specificity for serum albumin. More preferably, Y is a dAb (e.g., a V_{H}, V_{Λ}) that binds human serum albumin. In a particular embodiment, X or Z is human IL-1ra or a functional variant of human IL-1ra.

In certain embodiments, Y comprises an amino acid sequence selected from the group consisting of DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-8 (SEQ ID NO:746), DOM7r-13 (SEQ ID NO:747), and DOM7r-14 (SEQ ID NO:748). In other embodiments, Y comprises an amino acid sequence selected from the group consisting of DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), and DOM7h-27 (SEQ ID NO:745).

In certain embodiments, X and Z are independently a binding domain that has a binding site with binding specificity for IL-1R1. In some embodiments X and/or Z independently comprise an amino acid sequence selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

In other embodiments, the drug fusion comprises moieties X′ and Y′, wherein X′ is a polypeptide antagonist of IL-1R1, with the proviso that X′ does not comprise an antibody chain or a fragment of an antibody chain; and Y′ is a single chain antigen-binding fragment of an antibody that has binding specificity for serum albumin. Preferably, Y′ is an immunoglobulin heavy chain variable domain (V_{H}, V_{HH}) that has binding specificity for serum albumin, or an immunoglobulin light chain variable domain (V_{L}) that has binding specificity for serum albumin. More preferably, Y′ is a dAb (e.g., a V_{H}, V_{κ} or V_{λ}) that binds human serum albumin. X′ can be located amino terminally to Y′, or Y′ can be located amino terminally to X′. In some embodiments, X′ and Y′ are separated by an amino acid, or by a peptide or polypeptide linker that comprises from two to about 100 amino acids. In a particular embodiment, X′ is human IL-1ra or a functional variant of human IL-1ra.

In other embodiments, X′ is a binding domain that has a binding site with binding specificity for IL-1R1. In particular embodiments the antagonist of IL-1R1 fusion comprises a dAb that binds serum albumin and human IL-1ra (e.g., SEQ ID NO:786). Preferably, the dAb binds human serum albumin and comprises human framework regions. In some embodiments, X′ comprise an amino acid sequence selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

In certain embodiments, Y′ comprises an amino acid sequence selected from the group consisting of DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-8 (SEQ ID NO:746), DOM7r-13 (SEQ ID NO:747), and DOM7r-14 (SEQ ID NO:748). In other embodiments, Y′ comprises an amino acid sequence selected from the group consisting of DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), and DOM7h-27 (SEQ ID NO:745).

In other embodiments, the antagonist of IL-1R1 fusion or IL-1R1 conjugate comprises a functional variant of human IL-1ra that has at least about 80%, or at least about 85%, or at least about 90%, or at least about 95%, or at least about 96%, or at least about 97%, or at least about 98%, or at least about 99% amino acid sequence identity with the mature 152 amino acid form of human IL-1ra and antagonizes human Interleukin-1 type 1 receptor. (See, Eisenberg et al., Nature 343:341-346 (1990).) The IL-1ra variant can comprise one or more additional amino acids (e.g., comprise 153 or 154 or more amino acids).

In other embodiments, the antagonist of IL-1R1 fusion or IL-1R1 conjugate comprises a dAb that binds human IL-1R1 and inhibits a function of human IL-1 R1, and has an amino acid sequence that has at least about 80%, or at least about 85%, or at least about 90%, or at least about 95%, or at least about 96%, or at least about 97%, or at least about 98%, or at least about 99% amino acid sequence identity with the amino acid sequence of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

The antagonist of IL-1R1 fusions of the invention can be produced using any suitable method. For example, some embodiments can be produced by the insertion of a nucleic acid encoding the antagonist of IL-1R1 fusion into a suitable expression vector. The resulting construct can be introduced into a suitable host cell for expression. Upon expression, fusion protein can be isolated or purified from a cell lysate or preferably from the culture media or periplasm using any suitable method. (See e.g., Current Protocols in Molecular Biology (Ausubel, F. M. et al., eds., Vol. 2, Suppl. 26, pp. 16.4.1-16.7.8 (1991)).

Suitable expression vectors can contain a number of components, for example, an origin of replication, a selectable marker gene, one or more expression control elements, such as a transcription control element (e.g., promoter, enhancer, terminator) and/or one or more translation signals, a signal sequence or leader sequence, and the like. Expression control elements and a signal sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for procaryotic (e.g., lac, tac, T3, T7 promoters for E. coli) and eucaryotic (e.g., simian virus 40 early or late promoter, Rous sarcoma virus long terminal repeat promoter, cytomegalovirus promoter, adenovirus late promoter) hosts are available.

In addition, expression vectors typically comprise a selectable marker for selection of host cells carrying the vector, and, in the case of a replicable expression vector, an origin or replication. Genes encoding products which confer antibiotic or drug resistance are common selectable markers and may be used in procaryotic (e.g., lactamase gene (ampicillin resistance), Tet gene for tetracycline resistance) and eucaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, HIS3) are often used as selectable markers in yeast. Use of viral (e.g., baculovirus) or phage vectors, and vectors which are capable of integrating into the genome of the host cell, such as retroviral vectors, are also contemplated. Suitable expression vectors for expression in mammalian cells and prokaryotic cells (E. coli), insect cells (Drosophila Schnieder S2 cells, Sf9) and yeast (P. methanolica, P. pastoris, S. cerevisiae) are well-known in the art.

Antagonist of IL-1R1 fusions can be produced by the expression of a recombinant nucleic acid encoding the protein (e.g., an expression vector) in a suitable host cell, or using other suitable methods. For example, the expression constructs described herein can be introduced into a suitable host cell, and the resulting cell can be maintained (e.g., in culture, in an animal) under conditions suitable for expression of the constructs. The antagonist of IL-1R1 fusion can be isolated (e.g., from the culture media) if desired. Suitable host cells can be prokaryotic, including bacterial cells such as E. coli, B. subtilis and or other suitable bacteria, eucaryotic, such as fungal or yeast cells (e.g., Pichia pastoris, Aspergillus species, Saccharomyces cerevisiae, Schizosaccharomyces pombe, Neurospora crassa), or other lower eucaryotic cells, and cells of higher eucaryotes such as those from insects (e.g., Sf9 insect cells (WO 94/26087 (O'Connor)) or mammals (e.g., COS cells, such as COS-1 (ATCC Accession No. CRL-1650) and COS-7 (ATCC Accession No. CRL-1651), CHO (e.g., ATCC Accession No. CRL-9096), 293 (ATCC Accession No. CRL-1573), HeLa (ATCC Accession No. CCL-2), CV1 (ATCC Accession No. CCL-70), WOP (Dailey et al., J. Virol. 54:739-749 (1985)), 3T3, 293T (Pear et al., Proc. Natl. Acad. Sci. U.S.A., 90:8392-8396 (1993)), NSO cells, SP2/0, HuT 78 cells, and the like (see, e.g., Ausubel, F. M. et al., eds. Current Protocols in Molecular Biology, Greene Publishing Associates and John Wiley & Sons Inc., (1993)).

### Antagonist of IL-1R1 Conjugates

In another aspect, the invention provides conjugates comprising an antigen-binding fragment of an antibody that binds serum albumin that is bonded to an antagonist of IL-1R1. Such conjugates include “antagonist of IL-1R1 conjugates,” which comprise an antigen-binding fragment of an antibody that binds serum albumin to which an antagonist of IL-1R1 is covalently bonded, and “noncovalent antagonist of IL-1R1 conjugates,” which comprise an antigen-binding fragment of an antibody that binds serum albumin to which an antagonist of IL-1R1 is noncovalently bonded. Preferably, the conjugates are sufficiently stable so that the antigen-binding fragment of an antibody that binds serum albumin and antagonist of IL-1R1 remain substantially bonded (either covalently or noncovalently) to each other under in vivo conditions (e.g., when administered to a human). Preferably, no more than about 20%, no more than about 15%, no more than about 10%, no more than about 9%, no more than about 8%, no more than about 7%, no more than about 6%, no more than about 5%, no more than about 4%, no more than about 3%, no more than about 2%, no more than about 1% or substantially none of the conjugates dissociate or break down to release drug and antigen-binding fragment under in vivo conditions. For example, stability under “in vivo” conditions can be conveniently assessed by incubating drug conjugate or noncovalent drug conjugate for 24 hours in serum (e.g., human serum) at 37° C. In one example of such a method, equal amounts of a drug conjugate and the unconjugated drug are diluted into two different vials of serum. Half of the contents of each vial is immediately frozen at −20° C., and the other half incubated for 24 hours at 37° C. All four samples can then be analyzed using any suitable method, such as SDS-PAGE and/or Western blotting. Western blots can be probed using an antibody that binds the drug. All drug in the drug conjugate lanes will run at the size of the drug conjugate if there was no dissociation. Many other suitable methods can be used to assess stability under “in vivo” conditions, for example, by analyzing samples prepared as described above using suitable analytic methods, such as chromatography (e.g., gel filtration, ion exchange, reversed phase), ELISA, mass spectroscopy and the like.

### Covalent Antagonist of IL-1R1 Conjugates

In another aspect, the invention provides an antagonist of IL-1R1 conjugate comprising an antigen-binding fragment of an antibody that has binding specificity for serum albumin, and an antagonist of IL-1R1 that is covalently bonded to said antigen-binding fragment, with the proviso that the antagonist of IL-1R1 conjugate is not a single continuous polypeptide chain.

In some embodiments, the antagonist of IL-1R1 conjugate comprises an immunoglobulin heavy chain variable domain (V_{H}, V_{HH}) that has binding specificity for serum albumin, or an immunoglobulin light chain variable domain (V_{L}) that has binding specificity for serum albumin, and an antagonist of IL-1R1 moiety that is covalently bonded to said V_{H} or V_{L}, with the proviso that the antagonist of IL-1R1 conjugate is not a single continuous polypeptide chain. Preferably the antagonist of IL-1R1 conjugate comprises a single V_{H} that binds serum albumin or a single V_{L} that binds serum albumin. In certain embodiments, the antagonist of IL-1R1 conjugate comprises a Vₖ dAb that binds human serum albumin and comprises an amino acid sequence selected from the group consisting of DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-8 (SEQ ID NO:746), DOM7r-13 (SEQ ID NO:747), and DOM7r-14 (SEQ ID NO:748). In other embodiments, the antagonist of IL-1R1 conjugate comprises a V_{H} dAb that binds human serum albumin and comprises an amino acid sequence selected from the group consisting of DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), and DOM7h-27 (SEQ ID NO:745).

The antagonist of IL-1R1 conjugates can comprise any desired antagonist if IL-1R1 moiety (e.g., IL-1ra, functional variant of IL-1ra, dAb) and can be prepared using any suitable methods. For example, the antagonist of IL-1R1 moiety can be bonded to the antigen-binding fragment of an antibody that binds serum albumin directly or indirectly through a suitable linker moiety at one or more positions, such as the amino-terminus, the carboxyl-terminus or through amino acid side chains. In one embodiment, the antagonist of IL-1R1 conjugate comprises a dAb that binds human serum albumin and a polypeptide antagonists of TL-IR1 (e.g., human IL-1ra or a functional variant of human IL-1ra), and the amino-terminus of the polypeptide antagonists of IL-1R1 (e.g., human IL-1ra or a functional variant of human IL-1ra) is bonded to the carboxyl-terminus of the dAb directly or through a suitable linker moiety. In other embodiments, the conjugate comprises a dAb that binds human serum albumin and two or more different antagonists of IL-1R1 moieties are covalently bonded to the dAb. For example, a first antagonist of IL-1R1 moiety can be covalently bonded (directly or indirectly) to the carboxyl terminus of the dAb and a second antagonist of IL-1R1 moiety can be covalently bonded (directly or indirectly) to the amino-terminus or through a side chain amino group (e.g., E amino group of lysine). Such conjugates can be prepared using well-known methods of selective coupling. (See, e.g., Hermanson, G. T., Bioconjugate Techniques, Academic Press: San Diego, Calif. (1996).)

A variety of methods for conjugating antagonists of IL-1R1 to an antigen-binding fragment of an antibody that has binding specificity for serum albumin can be used. The particular method selected will depend on the antagonist of IL-1R1 to be conjugated. If desired, linkers that contain terminal functional groups can be used to link the antigen-binding fragment and the antagonist of IL-1R1. Generally, conjugation is accomplished by reacting an antagonist of IL-1R1 that contains a reactive functional group (or is modified to contain a reactive functional group) with a linker or directly with an antigen-binding fragment of an antibody that binds serum albumin. Covalent bonds form by reacting an antagonist of IL-1R1 that contains (or is modified to contain) a chemical moiety or functional group that can, under appropriate conditions, react with a second chemical group thereby forming a covalent bond. If desired, a suitable reactive chemical group can be added to the antigen-binding fragment or to a linker using any suitable method. (See, e.g., Hermanson, G. T., Bioconjugate Techniques, Academic Press: San Diego, Calif. (1996).) Many suitable reactive chemical group combinations are known in the art, for example an amine group can react with an electrophilic group such as tosylate, mesylate, halo (chloro, bromo, fluoro, iodo), N-hydroxysuccinimidyl ester (NHS), and the like. Thiols can react with maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)).

In some embodiments, the antigen-binding fragment of an antibody that has binding specificity for serum albumin is bonded to an antagonist of IL-1R1 moiety by reaction of two thiols to form a disulfide bond. In other embodiments, the antigen-binding fragment of an antibody that has binding specificity for serum albumin is bonded to an antagonist of IL-1R1 moiety by reaction of an isothiocyanate group and a primary amine to produce an isothiourea bond.

Suitable linker moieties can be linear or branched and include, for example, tetraethylene glycol, C₂-C₁₂ alkylene, —NH—(CH₂)ₚ—NH— or —(CH₂)ₚ—NH— (wherein p is one to twelve), —CH₂—O—CH₂—CH₂—O—CH₂—CH₂—O—CH—NH—, a polypeptide chain comprising one to about 100 (preferably one to about 12) amino acids and the like.

### Noncovalent Antagonist of IL-1R1 Conjugates

Some noncovalent bonds (e.g., hydrogen bonds, van der Waals interactions) can produce stable, highly specific intermolecular connections. For example, molecular recognition interactions achieved through multiple noncovalent bonds between complementary binding partners underlie many important biological interactions, such as the binding of enzymes to their substrates, the recognition of antigens by antibodies, the binding of ligands to their receptors, and stabilization of the three dimensional structure of proteins and peptide. Accordingly, such weak noncovalent interactions (e.g., hydrogen bonding, van Der Waals interactions, electrostatic interactions, hydrophobic interactions and the like) can be utilized to bind an antagonist of IL-1R1 to the antigen-binding fragment of an antibody that has binding specificity for serum albumin.

Preferably, the noncovalent bond linking the antigen-binding fragment and antagonist of IL-1R1 be of sufficient strength that the antigen-binding fragment and antagonist of IL-1R1 remain substantially bonded to each under in vivo conditions (e.g., when administered to a human). Generally, the noncovalent bond linking the antigen-binding fragment and antagonist of IL-1R1 has a strength of at least about 10¹⁰ M⁻¹. In preferred embodiments, the strength of the noncovalent bond is at least about 10¹¹ M⁻¹, at least about 10¹² M⁻¹, at least about 10¹³ M⁻¹, at least about 10¹⁴ M⁻¹ or at least about 10¹⁵ M⁻¹. The interactions between biotin and avidin and between biotin and streptavidin are known to be very efficient and stable under many conditions, and as described herein noncovalent bonds between biotin and avidin or between biotin and streptavidin can be used to prepare a noncovalent antagonist of IL-1R1 conjugate.

The noncovalent bond can be formed directly between the antigen-binding fragment of an antibody that has a specificity for serum albumin and antagonist of IL-1R1, or can be formed between suitable complementary binding partners (e.g., biotin and avidin or streptavidin) wherein one partner is covalently bonded to antagonist of IL-1R1 and the complementary binding partner is covalently bonded to the antigen-binding fragment. When complementary binding partners are employed, one of the binding partners can be covalently bonded to the antagonist of IL-1R1 directly or through a suitable linker moiety, and the complementary binding partner can be covalently bonded to the antigen-binding fragment of an antibody that binds serum albumin directly or through a suitable linker moiety.

Complementary binding partners are pairs of molecules that selectively bind to each other. Many complementary binding partners are known in the art, for example, antibody (or an antigen-binding fragment thereof) and its cognate antigen or epitope, enzymes and their substrates, and receptors and their ligands. Preferred complementary binding partners are biotin and avidin, and biotin and streptavidin.

Direct or indirect covalent bonding of a member of a complementary binding pair to an antigen-binding fragment that has binding specificity for serum albumin or an antagonist of IL-1R1 can be accomplished as described above, for example, by reacting a complementary binding partner that contains a reactive functional group (or is modified to contain a reactive functional group) with an antigen-binding fragment of an antibody that binds serum albumin, with or without the use of a linker. The particular method selected will depend on the compounds (e.g., antagonist of IL-1R1, complementary binding partner, antigen-binding fragment of an antibody that binds serum albumin) to be conjugated. If desired, linkers (e.g., homobifunctional linkers, heterobifunctional linkers) that contain terminal reactive functional groups can be used to link the antigen-binding fragment and/or the antagonist of IL-1R1 to a complementary binding partner. In one embodiment, a heterobifunctional linker that contains two distinct reactive moieties can be used. The heterobifunctional linker can be selected so that one of the reactive moieties will react with the antigen-binding fragment of an antibody that has binding specificity for serum albumin or the antagonist of IL-1R1, and the other reactive moiety will react with the complementary binding partner. Any suitable linker (e.g., heterobifunctional linker) can be used and many such linkers are known in the art and available for commercial sources (e.g., Pierce Biotechnology, Inc., IL).

### EXAMPLES

### Example 1

### Immunoglobulin Variable Domain Antagonists of IL-1R1

### Methods

### Selections and Screening

For primary selections, 4G-K2 library of V_{κ} dAbs was panned against IL-1R1-Fc fusion protein (Axxora, Nottingham, UK). Domain antibodies from the primary selection were subjected to three further rounds of selection. Round 1 was performed using protein G coated magnetic beads (Dynal, Norway) and 100 nM IL-1R1-Fc; round 2 was performed using anti-human IgG beads (Novagen, Merck Biosciences, Nottingham, UK) and 10 nM IL-1R1-Fc; and round 3 was performed using protein G beads and 1 nM IL-1R1-Fc. (Henderikx et al., Selection of antibodies against biotinylated antigens. Antibody Phage Display: Methods and protocols, Ed. O'Brien and Atkin, Humana Press (2002).) Elution at each stage was with 1 mg/ml trypsin-PBS. For affinity maturation selections, the above method was used, but with the following modifications: two rounds of selection were performed using protein G beads, round 1 using 1 nM IL-1R1-Fc, and round 2 using 100 pM IL-1R1-Fc. Phage vectors from selection outputs (rounds 2 and 3) were isolated by plasmid preps (Qiagen) and dAb inserts were released by restriction digest with Sal I and Not I. This inserts were ligated into a phage expression vector (Sal I/Not I cut pDOM5) and used to transform E. coli strain HB2151 for soluble expression and screening of dAbs.

### Supernatant Receptor Binding Assay (RBA)

Single transformed E. coli colonies were picked into 96-well plates containing 2×TY supplemented with 100 μg/ml carbenicillin and 0.1% (w/v) glucose, grown at 37° C. to ˜OD₆₀₀=0.9 and induced with 1 mM IPTG. Supernatants from overnight inductions at 30° C. were screened in a receptor binding assay for the ability to inhibit binding of IL-1β to IL-1RI captured on an ELISA plate. Briefly, MaxiSorp™ immunoassay plates (Nunc, Denmark) were incubated overnight with anti-IL-1RI mouse monoclonal antibody (R&D Systems, Minneapolis, USA). The wells were washed with phosphate buffered saline (PBS) containing 0.1% (v/v) Tween-20 and then blocked with 1% (w/v) BSA in PBS before being incubated with recombinant IL-1RI (500 ng/ml, R&D Systems). The E. coli culture supernatants containing dAbs to be screened were placed in the washed wells of the assay plate, the plate was incubated for 30 min, then IL-1β (4 ng/ml, R&D Systems) was added to each well and mixed. IL-1β binding was detected using biotinylated anti-IL-1β antibody (R&D Systems), followed by peroxidase labelled anti-biotin antibody (Stratech, Soham, UK) and then, incubation with 3,3′,5,5′-tetramethylbenzidine (TMB) substrate (KPL, Gaithersburg, USA). The reaction was stopped by the addition of HCl and the absorbance was read at 450 nm. Anti-IL-1RI dAb activity caused a decrease in IL-1β binding and therefore a decrease in absorbance compared with the IL-1β only control.

### Cell Assay

Isolated dAbs were tested for their ability to inhibit IL-1-induced IL-8 release from cultured MRC-5 cells (ATCC catalogue no. CCL-171). Briefly, 5000 trypsinised MRC-5 cells in RPMI media were placed in the well of a tissue-culture microtitre plate and mixed with IL-1α or β (R&D Systems, 200 pg/ml final concentration) and a dilution of the dAb to be tested. The mixture was incubated overnight at 37° C. and IL-8 released by the cells into to culture media was quantified in an ELISA (DuoSet®, R&D Systems). Anti-IL-1RI dAb activity caused a decrease in IL-1 binding and a corresponding reduction in IL-8 release.

### Human Whole Blood Assay

Whole human blood was incubated with a dilution series of the dAb to be tested, and the mixture was incubated for 30 min at 37° C./5% CO₂. Next, 270 or 900 pM (final concentration) IL-1α or IL-1β was added and the mixture, and then the mixtures was incubated at 37° C./5% CO₂ for an additional 20 hours. The blood was then centrifuged (500×g, 5 min) and the IL-6 released into the supernatant was quantified in an ELISA (DuoSet®, R&D Systems). Anti-IL-1RI dAb activity caused a decrease in IL-1 binding and a corresponding reduction in IL-6 release.

### Off-Rate Screening

These experiments were performed on a BIACORE 3000 surface plasmon resonance instrument, using a CM5 chip (Biacore) coupled with ˜600 RU of IL-1RI (R&D Systems). Analytes were passed over the IL-1RI-coated flow-cell, with in-line referencing against a blank flow-cell, at a flow rate of 30 μl/min in HBS-EP running buffer (Biacore). Ten microlitres of supernatant containing soluble dAb was diluted 1:1 in running buffer, injected (Kinject) at 101/min flow rate and allowed to dissociate in buffer. Clones with improved off-rates compared to parental clones were identified by eye, or by measurement using BIAevaluation software v4.1.

### Affinity Maturation Phage Library Construction

Two types of libraries were constructed: CDR-re-diversified libraries and error-prone libraries. For the former type of library, PCR reactions were performed, using degenerate oligonucleotides containing NNK or NNS codons, to diversify the required positions in the dAb to be affinity matured. Assembly PCR was then used to generate a full length diversified insert. For the error-prone library, plasmid DNA encoding the dAb to be affinity matured was amplified by PCR, using the GeneMorph® II Random Mutagenesis kit (Stratagene). Inserts produced by either method were digested with Sal I and Not I and used in a ligation reaction with cut phage vector. This ligation was then used to transform E. coli strain TB1 by electroporation and the transformed cells were plated on 2×TY agar containing 15 μg/ml tetracycline, yielding library sizes of >1×10⁸ clones.

### Results

### Primary Selection and Screening

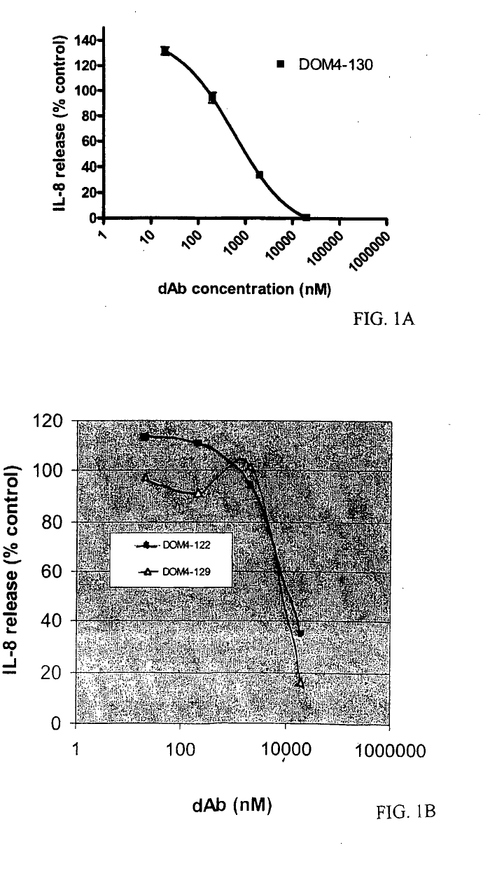

Primary phage selections were performed using the 4G-K2 library and outputs sub-cloned into a soluble expression vector. dAb clones that inhibit binding of IL-1 to IL-1RI were identified by supernatant RBA (results not shown), then expressed, purified by protein L and tested for their ability to inhibit IL-1-induced IL-8 release in an MRC-5 cell assay. FIG. 1A shows a typical dose-response curve for anti-IL-1RI dAb referred to as DOM4-130 in such a cell assay. The ND₅₀ of DOM4-130 in this assay was approximately 500-1000 nM. FIG. 1B shows a dose-response curve for anti-IL-1RI dAbs referred to as DOM4-122 and DOM4-129 in such a cell assay. The ND₅₀ values of both dAbs was about 1 μM. DOM4-122 and DOM4-129 have the same amino acid sequence in CDRs 1 and 2, and have two out of five amino acid residues identical in CDR3, and therefore were predicted to bind to the same epitope (have the same epitopic specificity) on IL-1R1.

### Affinity Maturation

### DOM4-130

### Stage I Maturation

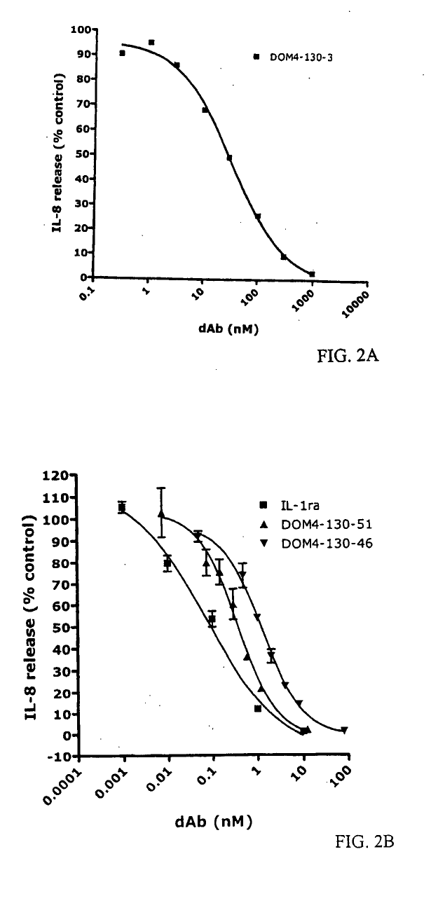

Using DOM4-130 as a template, a maturation library was constructed with diversity encoding all 20 amino acids at positions 30, 34, 93 and 94. The resulting phage library was used in soluble selections for binding to IL-1R1 using IL-1RI-Fc. Round 2 selection output was cloned into phage expression vector (pDOM5), dAbs were expressed in E. coli, and the expression supernatants were screened for improved off-rates compared to parental dAb. Clones with improved off-rates were expressed, purified and tested in the MRC-5/IL-8 assay. FIG. 2A depicts a dose-response curve for improved variant DOM4-130-3, which had an ND₅₀ of about 30 nM.

### Stage II Maturation

Using DOM4-130-3 as template, a maturation library was constructed as described above, except this time diversity was introduced at amino acid residues 49, 50, 51 and 53 in CDR2. The resulting library was again screened for variants with improved off-rates, which were tested in the MRC-5/IL-8 cell assay. FIG. 2B depicts a dose-response curve for improved clone DOM4-130-46 (ND₅₀ about 1 nM), together with an additional variant, DOM4-130-51. DOM4-130-51 was derived from DOM4-130-46, with the mutation S67Y added to improve potency further (ND₅₀ about 300 pM). Further variants of both of these dAbs were produced by introducing the amino acid replacement R107K, to revert the amino acid sequence to the germline sequence at this position, generating DOM4-130-53 and DOM4-130-54, respectively.

### DOM4-122 and DOM4-129

### Stage I Maturation

Using DOM4-122 as a template, a maturation library was constructed with diversity encoding all 20 amino acids at positions 28, 30, 31, 92 and 93. In parallel, DOM4-129 was affinity matured by error-prone PCR mutagenesis. The resulting phage libraries were used in soluble selections for binding to IL-1R1 using IL-1RI-Fc. Round 2 and 3 selection outputs were cloned into phage expression vector (pDOM5), dAbs were expressed in E. coli, and expression supernatants screened for improved off-rates compared to parent. Clones with improved off-rates were expressed, purified and tested in the MRC-5/IL-8 assay. FIG. 3 depicts a dose-response curve for improved variant DOM4-122-6 and DOM4-129-1, which both had an ND₅₀ value of about 10 nM.

### Stage II Maturation

DOM4-129-1 and DOM4-122-6 gained an amino acid replacement, L46F, in common during maturation. DOM4-129-1 has an additional amino acid replacement, S56R. Both changes were frequently found in clones isolated from maturation selections, therefore the S56R replacement was introduced into DOM4-122-6, yielding DOM4-122-23. DOM4-122-23 had an ND₅₀ of approximately 1 nM. An additional amino acid replacement, K45M, gained in both DOM4-122 and DOM4-129 was shown to be non-essential when reverted to the germline amino acid in DOM4-122-23, yielding DOM4-122-24.

### Example 2

### Antagonists of IL-1R1 are Efficacious in a Subchronic Model of COPD in C57BL/6 Mice

In this study, an antagonist of IL-1R1 (and extended half-life fusion protein comprising IL-1ra and a dAb that binds mouse serum albumin), was administered alone or in combination with an antagonists of TNFR1 by the intra-peritoneal injection every 48 hours beginning 24 hours prior to the initial tobacco smoke (TS) exposure. The effects on TS-induced changes in pulmonary inflammatory indices induced by 11 consecutive daily TS exposures were examined 24 hours following the final exposure. The results demonstrate that the antagonist of IL-1R1 was efficacious in the mouse model. ENBREL® (etanercept; Immunex Corporation), which binds TNF and thereby antagonizes TNFR1, was included as a comparator.

Test Compound 1: ENBREL® (etanercept; Immunex Corporation)
Test Compound 2: IL-1 ra/anti-SA dAb (IL-1ra fused to DOM7 m16)
Test Compound 3: 1:1 mixture of PEG DOM1m (anti-TNFR1 dAb comprise an 40 kDa branched polyethylene glycol moiety, TAR2m-21-23) and IL-1ra/anti-SA dAb. For all test substances, the vehicle was sterile saline. Dose volume was 10 ml/kg for test substances 1-3 and 20 ml/kg for test substance 4

The amino acid sequence of IL-1ra/anti-SA dAb is

(SEQ ID NO:787)

RPSGRKSSKMQAFRIWDVNQKTFYLRNNQLVAGYLQGPNVNLEEKIDVVP
IEPHALFLGIHGGKMCLSCVKSGDETRLQLEAVNITDLSENRKQDKRFAF
IRSDSGPTTSFESAACPGWFLCTAMEADQPVSLTNMPDEGVMVTKFYFQE
DESSGGGGSGGGGSGGGGSGGGGSGGGGSTDIQMTQSPSSLSASVGDRVT
ITCRASQSIIKHLKWYQQKPGKAPKLLIYGASRLQSGVPSRFSGSGSGTD
FTLTISSLQPEDFATYYCQQGARWPQTFGQGTKVEIKR

### Methods

Female mice (C57BL/6) full barrier bred and certified free of specific micro organisms on receipt (16-20 g) (Charles River) were housed in groups of up to 5 in individually ventilated, solid bottomed cages (IVC) with aspen chip bedding. Environments (airflow, temperature and humidity) within the cages were controlled by the IVC system (Techniplast).

There were 5 treatment groups, groups 1-4 contained 10 animals and group 5 contained 5 animals. The treatment groups are summarized in Table 1. All treatments were administered intraperatoneally, and the dose volume for groups 1-4 was 10 ml/kg and was 20 ml/kg for group 5. Treatments were administered every 48 hours, and the initial dose was administered 24 hours prior to the initial TS or air exposure. Subsequent treatment doses were administered 1 hour prior to each TS or air exposure.

TABLE 1

| Group | TS/Air | Compound | Dose |
|---|---|---|---|
| No. | Exposure | No. | mg/kg |
| 1 | Sham | Vehicle | 0 |
| 2 | TS | Vehicle | 0 |
| 3 | TS | 1 | 10 |
| 4 | TS | 2 | 10 |
| 5 | TS | 3 | 20 |

### TS Exposure

Mice (maximum 5 per exposure chamber) were exposed to TS generated from cigarettes (Type 1R1, supplied by University of Kentucky). Initial exposure was to 4 cigarettes on day 1, increasing to a maximum of 6 cigarettes per day by day 6/7. Exposure thereafter to day 11 was 6 cigarettes/day. The rate of increase was regulated with regard to the daily observed tolerance of the mice. The control group of mice was exposed to air for an equivalent length of time on each exposure day (air exposure controls).

### Health Monitoring:

Animals were weighed prior to the start of the study, on day 6 of the exposure protocol, and at the time of termination. All animals were monitored during and after each test substance administration and TS exposure.

### Terminal Procedures:

Animals were sacrificed by anaesthetic overdose (pentobarbitone Na, 100 mg/kg i.p.) as follows: All groups were sacrificed 24 hours after the 11^{th} and final TS exposure. Mice from all treatment groups were treated as follows: Blood samples were taken from the sub-clavian artery, placed in a microcentrifuge tube and allowed to clot overnight at 4° C. The clot was removed and the remaining fluid was centrifuged at 2900 rpm in a microcentrifuge for 6 minutes. The resulting supernatant serum was decanted and stored at −40° C. for possible PK analysis. A bronchoalveolar lavage (BAL) was performed using 0.4 ml of phosphate buffered saline (PBS). Cells recovered from the BAL were quantified by total and differential cell counts. Lungs were removed, snap frozen in liquid nitrogen and stored at −80° C. for possible PK analysis

### Data Analysis

A test for normality was carried out on the data. If the test was positive, then a preliminary analysis was carried out using a one way analysis of variance test (one way ANOVA) followed by a Bonferroni's multiple comparison post test to compare control and treatment groups. If the data was not normally distributed, then a Kruskal-Wallis test followed by Dunn's multiple comparisons test was employed. Data were considered significant when p<0.05.

### Results

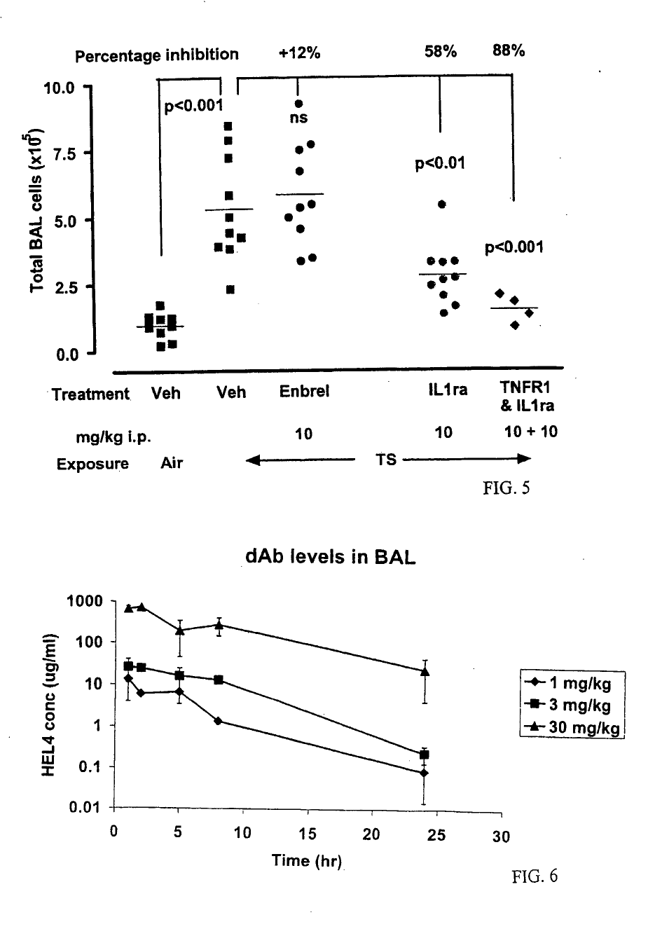

The IL-1ra/SA dAb treatment groups, show significantly reduced cell infiltrates in the lung compared to the TS exposed and vehicle treated control group (FIG. 5). The level of cells in the lung was reduced by 58% for total cells (p<0.01), 56% for macrophages (p<0.001), 59% for polymorphic nuclear cells (p<0.01), 70% for eosinophils p<0.01), and 65% for lymphocytes (p<0.01). A 29% reduction in epithelial cells was observed but this change was not significant.

The combination treatment group with IL1ra/SA dAb and PEGylated anti-TNFR1 dAb, show significantly reduced cell infiltrates in the lung. 88% inhibition for total cells (p<0.001), 82% for macrophages, 94% for epithelial cells, 93% for polymorphic nuclear cells, 93% for cosinophils and 86% for lymphocytes.

No significant reductions in any of the cell populations were observed in the ENBREL® (etanercept; Immunex Corporation) treated group. ENBREL® (etanercept; Immunex Corporation) even led to an increased number of total cells, although the increase was not statistically significant (FIG. 5).

### Example 3

### Local Administration of an Immunoglobulin Variable Domain to Pulmonary Tissue

In this study, an domain antibody (V_{H}) that binds hen egg lysozyme was administered locally to pulmonary tissue by intranasal administration, and pharmacokinetics were determined. The results demonstrate that domain antibodies can be delivered locally to pulmonary tissue model.

### Methods

Female mice (C57BL/6) full barrier bred and certified free of specific micro organisms on receipt (16-20 g) (Charles River) were housed in groups of up to 5 in individually ventilated, solid bottomed cages (IVC) with aspen chip bedding. Environments (airflow, temperature and humidity) within the cages were controlled by the IVC system (Techniplast).

The domain antibody HEL4 is a V_{H} that binds Hen egg lysozyme. (See, Jespers et al. J. Mol. Biol., 337:893-903 (2004). HEL-4 monomer (12 mg/ml) which contained an HA tag for detection was diluted in 20 mM sodium citrate pH 6.0, 100 mM NaCl. Mice were lightly anaesthetised (Isofluorane/O₂) and 50 microliters of dAb solution or vehicle control was dropped gently onto the nares. The animals were held in an upright position for a few seconds while spontaneously breathing in the solution before being allowed to recover and returned to their cages.

### Treatment Groups

There were 17 groups, the groups administered HEL-4 each contained 3 mice, while the vehicle control groups each contained two mice. The dose volume was 50 μl (25 μl/nare), and all mice were treated on the same day. Mice were sacrificed 1, 2, 5, 8 or 24 hours after treatment was administered (8 hours and 24 hours after treatment for vehicle groups). The study protocol is summarized in Table 2.

TABLE 2

| | | | | Concentra- | | Sacrifice time |
|---|---|---|---|---|---|---|
| Group | | | | tion | | after |
| No. | Treatment | Dose | | mg/ml | | administration |
| 1 | HEL-4 | 30 | mg/kg | 12 | mg/ml | 1 |
| 2 | HEL-4 | 30 | mg/kg | 12 | mg/ml | 2 |
| 3 | HEL-4 | 30 | mg/kg | 12 | mg/ml | 5 |
| 4 | HEL-4 | 30 | mg/kg | 12 | mg/ml | 8 |
| 5 | HEL-4 | 30 | mg/kg | 12 | mg/ml | 24 |
| 6 | HEL-4 | 3 | mg/kg | 1.2 | mg/ml | 1 |
| 7 | HEL-4 | 3 | mg/kg | 1.2 | mg/ml | 2 |
| 8 | HEL-4 | 3 | mg/kg | 1.2 | mg/ml | 5 |
| 9 | HEL-4 | 3 | mg/kg | 1.2 | mg/ml | 8 |
| 10 | HEL-4 | 3 | mg/kg | 1.2 | mg/ml | 24 |
| 11 | HEL-4 | 1 | mg/kg | 0.4 | mg/ml | 1 |
| 12 | HEL-4 | 1 | mg/kg | 0.4 | mg/ml | 2 |
| 13 | HEL-4 | 1 | mg/kg | 0.4 | mg/ml | 5 |
| 14 | HEL-4 | 1 | mg/kg | 0.4 | mg/ml | 8 |
| 15 | HEL-4 | 1 | mg/kg | 0.4 | mg/ml | 24 |
| 16 | Vehicle | 50 | μl/mouse | 0 | | 8 |
| 17 | Vehicle | 50 | μl/mouse | 0 | | 24 |

### Health Monitoring

Animals were weighed prior to the start of the study. All animals were monitored during and after each administration. Animals in the 24 hour groups were monitored at regular intervals overnight.

### Terminal Procedures

Animals were sacrificed by anaesthetic overdose (pentobarbitone Na, 100 mg/kg i.p.). Blood was taken from the subclavian artery, placed in a microcentrifuge tube and allowed to clot overnight at 4° C. The clot was gently removed and the remaining fluid was centrifuged at 2900 rpm in a microcentrifuge for 6 minutes. The resulting supernatant was decanted, placed in a fresh tube, frozen and stored at −40° C. prior to analysis. Bronchoalveolar lavage (BAL) was conducted using 0.4 ml of phosphate buffered saline (PBS) which was instilled and withdrawn 3 times. The BAL was centrifuged at 2700 rpm in a microcentrifuge for 6 minutes and the supernatant removed and stored at −40° C. prior to analysis. The cell pellet was re-suspended in a suitable volume of PBS and total cell count determined using a haemocytometer. Cytospin slides were prepared for differential cell determinations. The lungs were excised, snap frozen and stored at −80° C. prior to analysis. Using a mortar and pestle lungs were pulverized under liquid nitrogen and dissolved in T-PER® Tissue Protein Extraction Reagent (Pierce) and homogenized using 40 strokes with a dounce homogenizer.

### ELISA to Detect HA Tagged HEL-4

A 96 well Maxisorp (Nunc) assay plate was coated overnight at 4° C. with 100 μl per well of goat polyclonal anti HA tag antibody (Abcam) at 2 μg/ml in carbonate buffer. Wells were washed 3 times with 0.05% tween/PBS and 3 times with PBS. 200 μl per well of 2% BSA in PBS was added to block the plate. After blocking, wells are washed and then 100 μl of HA tagged dAb standard or sample was added. Wells were washed and then 100 μl Protein A—HRP (1:5000 dilution; Amersham) was added to each well. Plates were developed by adding 100 μl of SureBlue 1-Component TMB MicroWell Peroxidase (KPL, Gaithersburg, USA) solution to each well, and the plate was left at room temperature until a suitable signal has developed. The reaction was stopped by the addition of HCl and absorbance was read at 450 nm.

### Results

The total BAL cell counts showed that administering HEL-4 domain antibody at doses of 1, 3 or 30 mg/kg did not cause significant inflammation in the lungs. Some of the animals had increased cellular infiltrates but these were not significantly different from animals treated with vehicle alone. The HEL-4 levels in the BAL show that the dAbs are delivered efficiently into the deep lung (FIG. 6). A dose related effect was observed. At 2 hours after administration, a maximum level of 700 ug/ml was detected in the lung with the 30 mg/kg dosing. Thus, about 48% (280 μg of 600 μg total delivered) of the administered material was recovered from the lung, which means that more than 48% material that was administered was delivered to the lung but not all dAb delivered to the lung can be recovered, or is present in the surrounding tissues. The levels in the BAL are high for a prolonged period of time and there appears to be a slow release into the surrounding tissues.

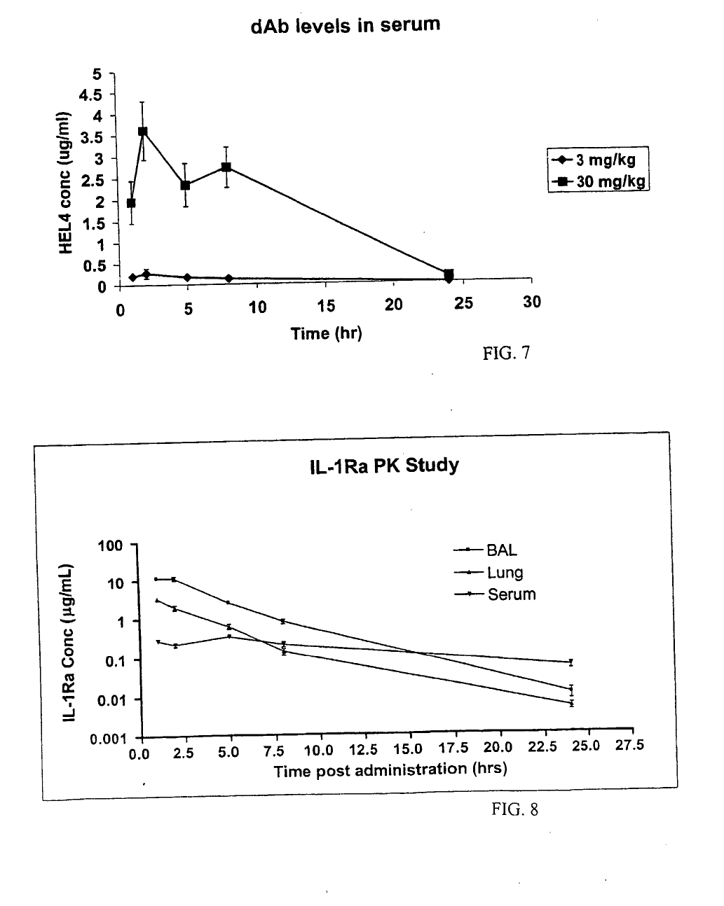

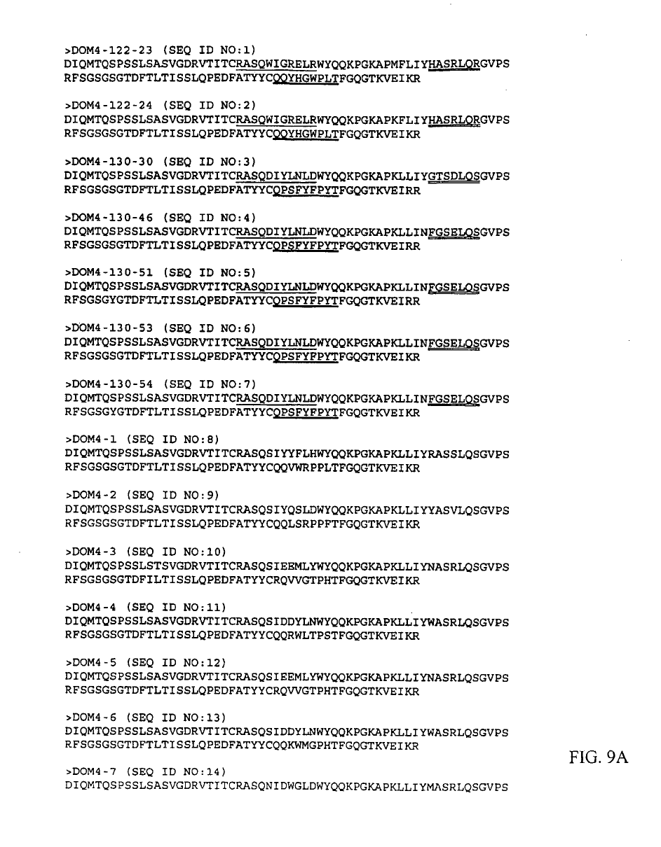

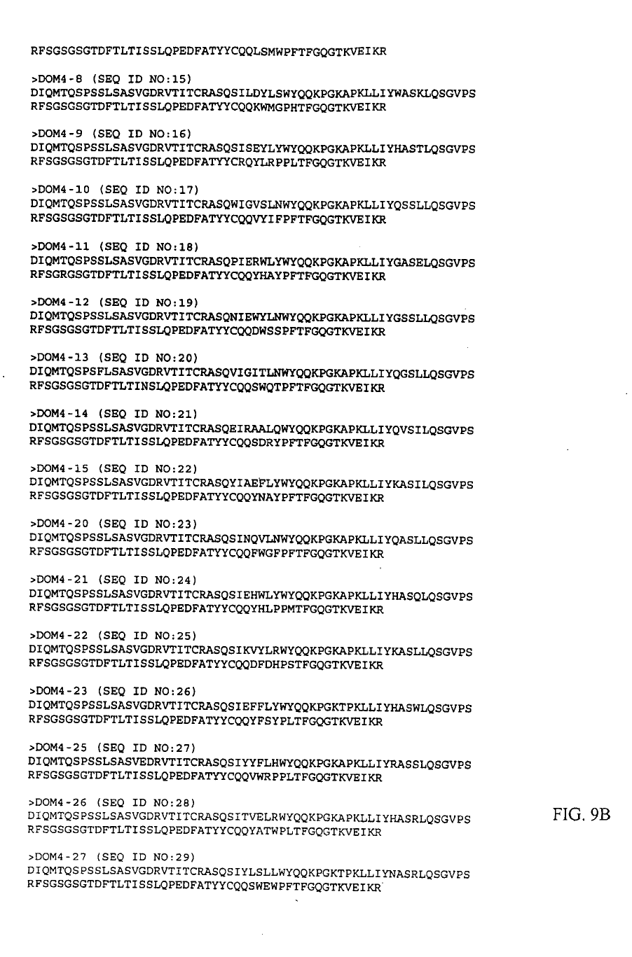

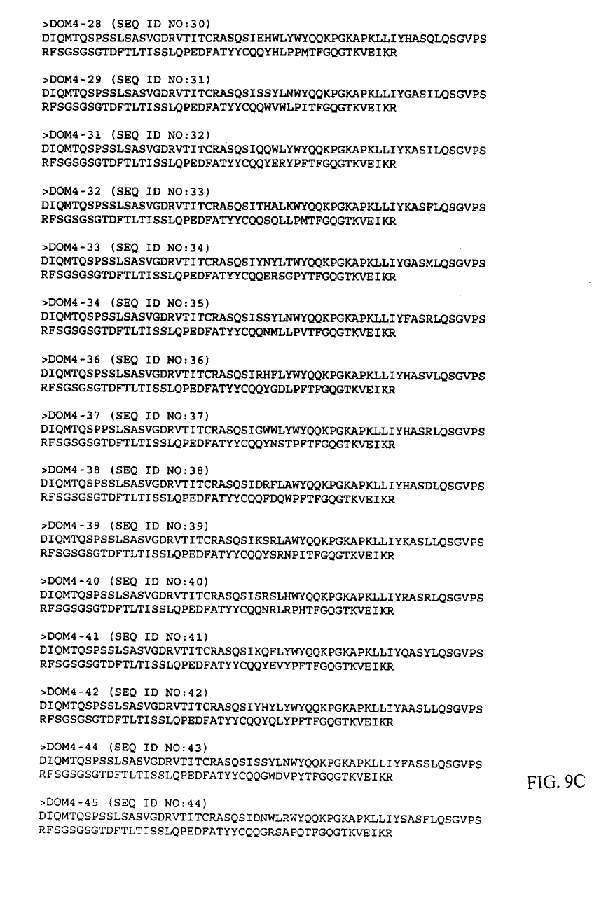

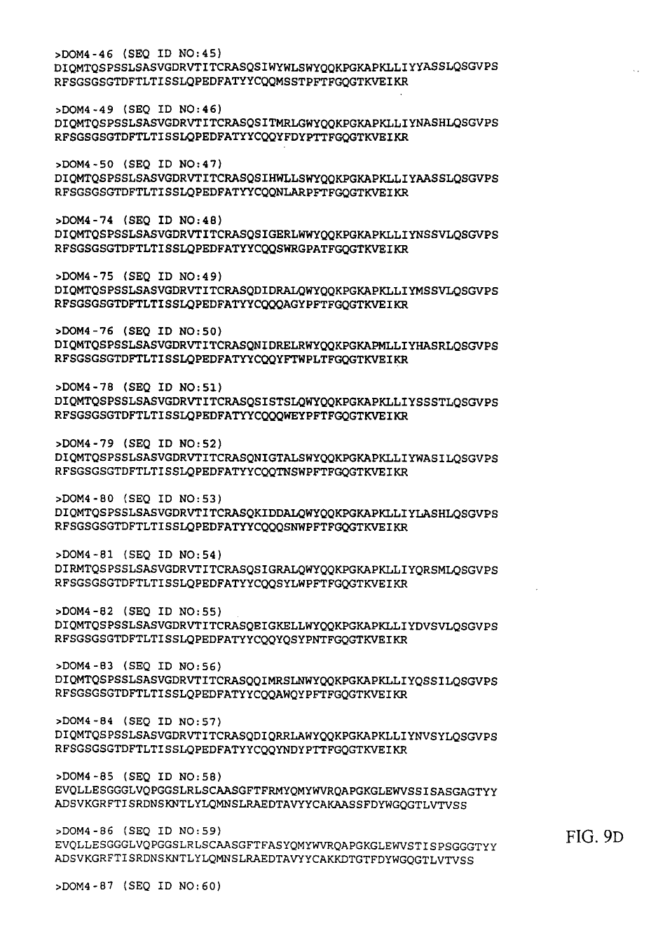

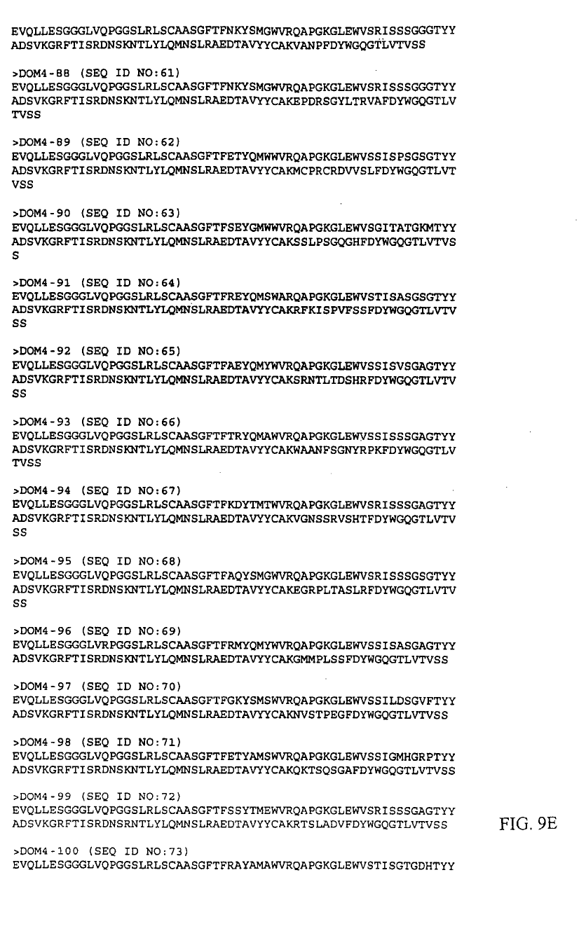

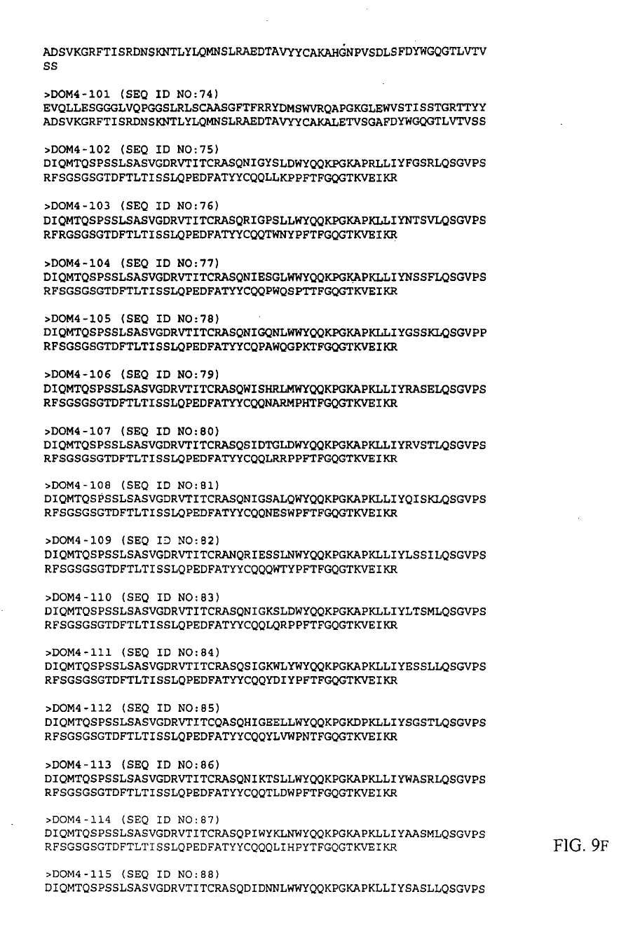

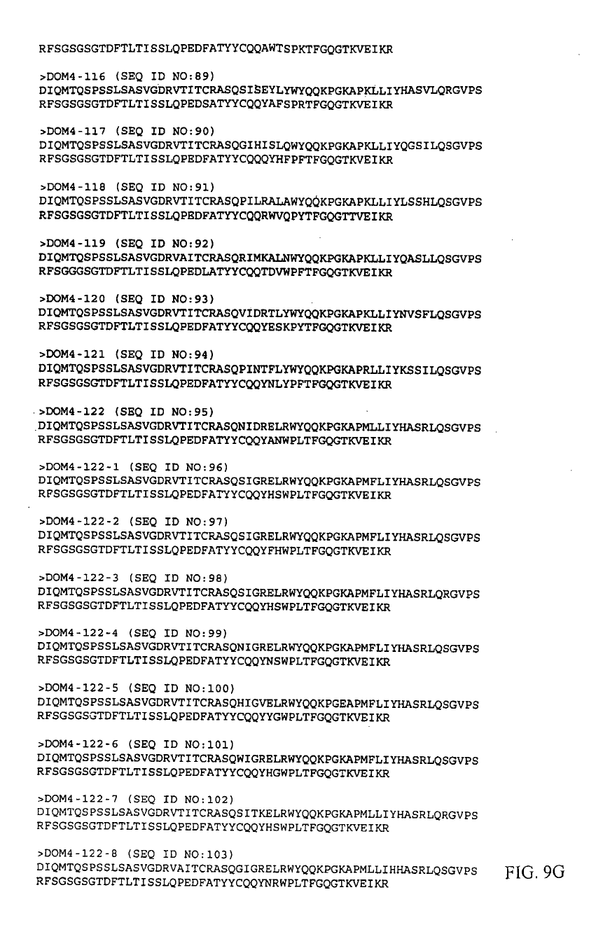

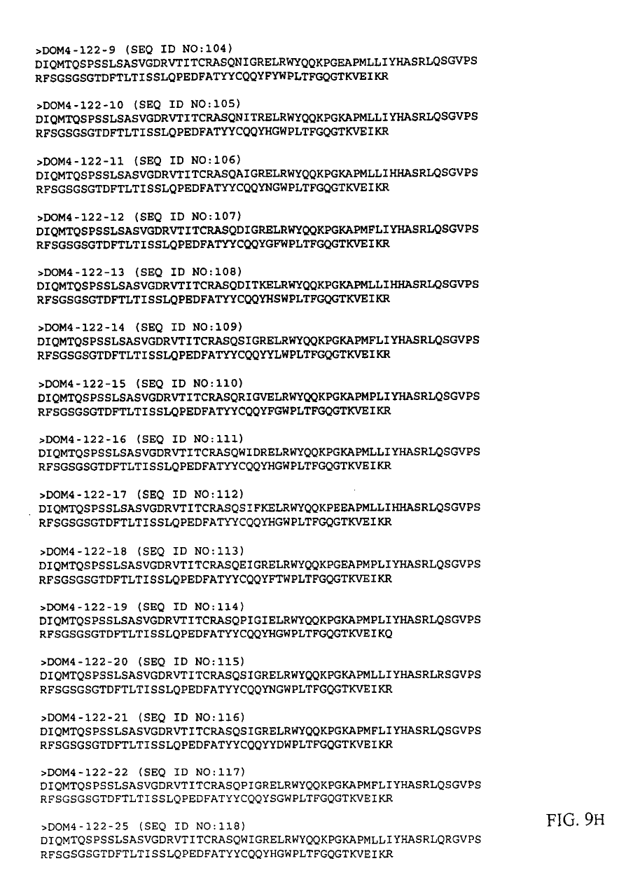

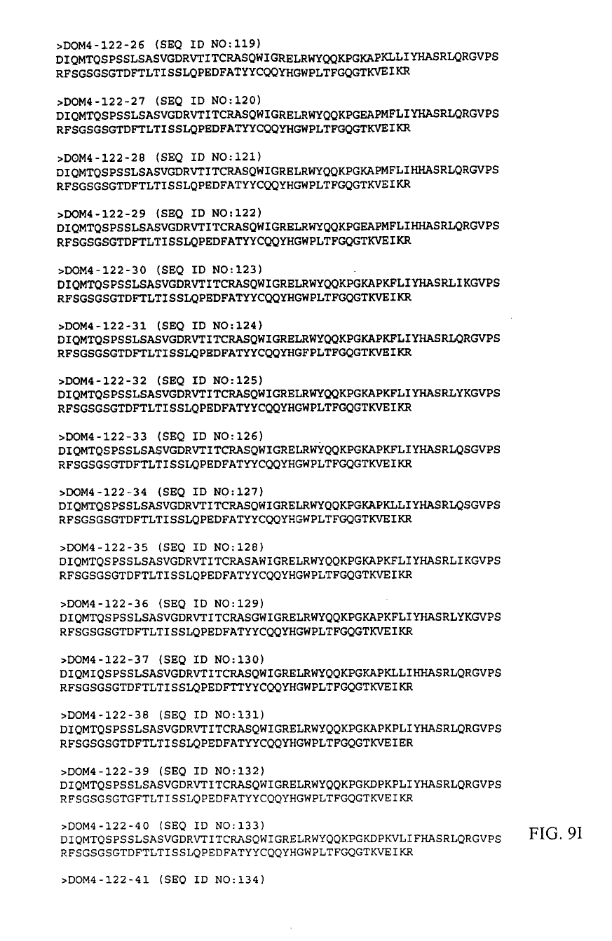

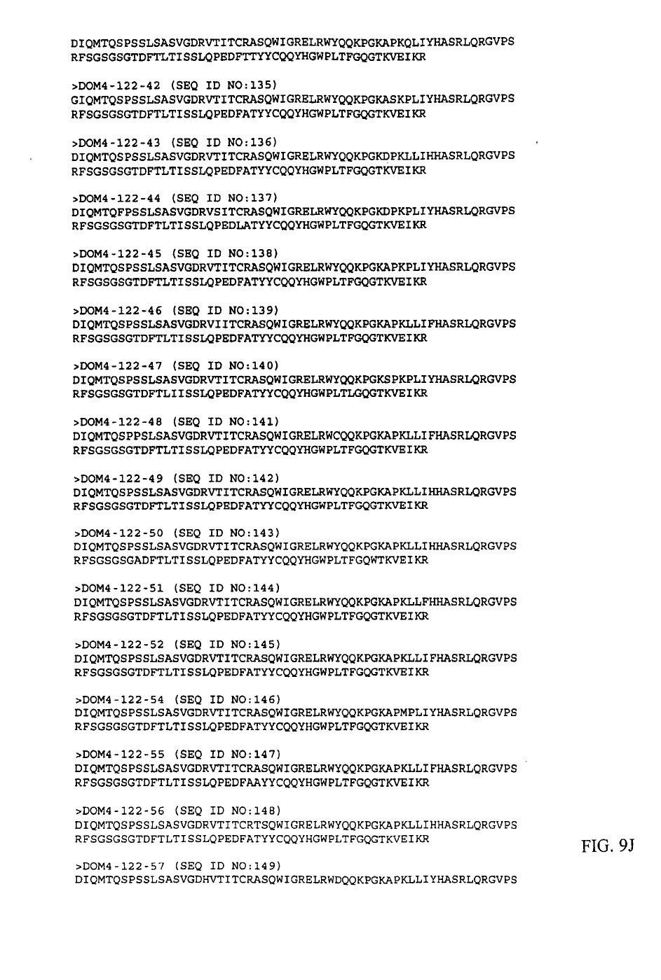

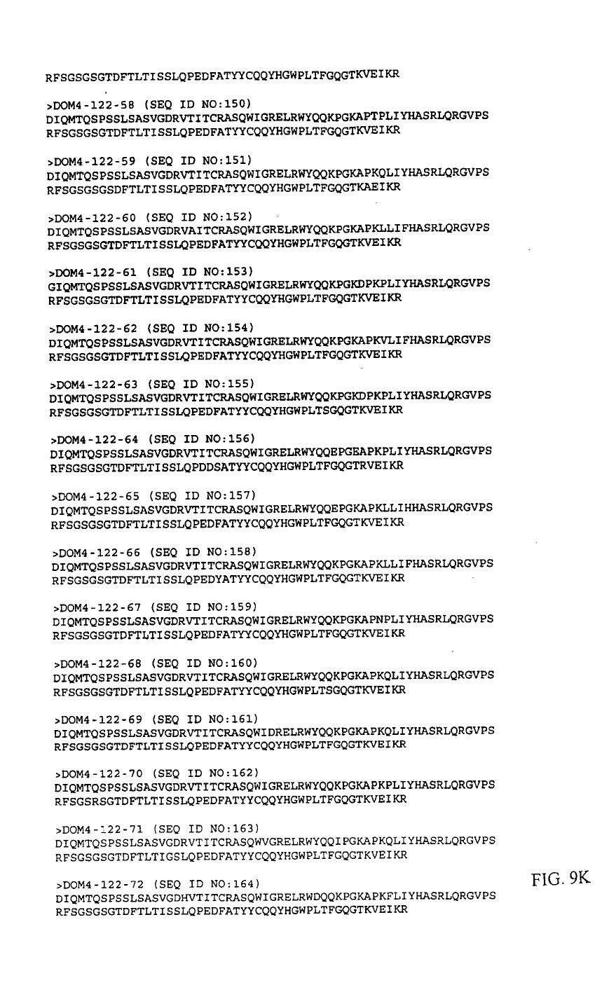

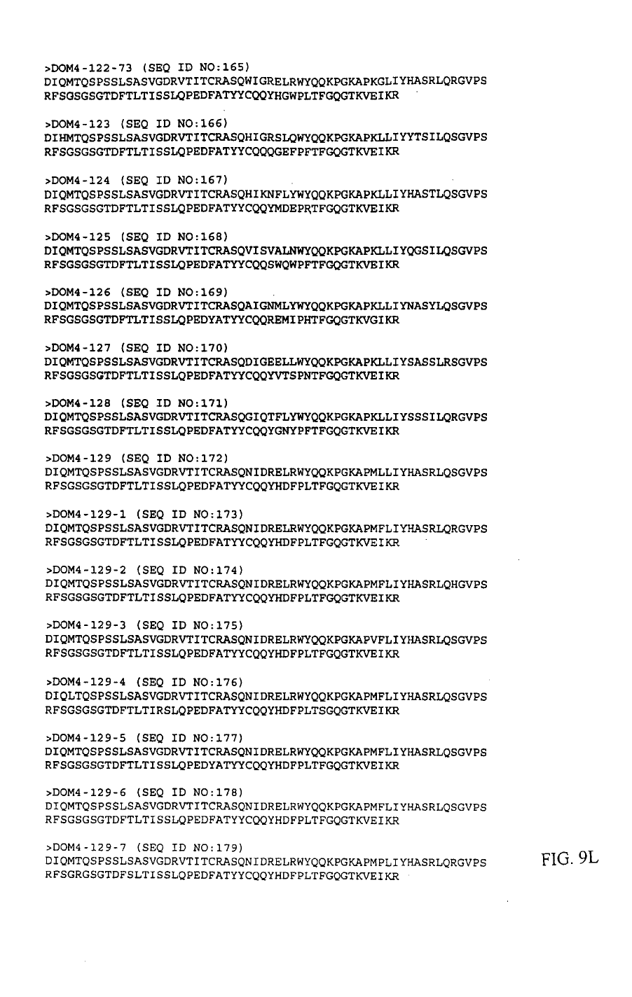

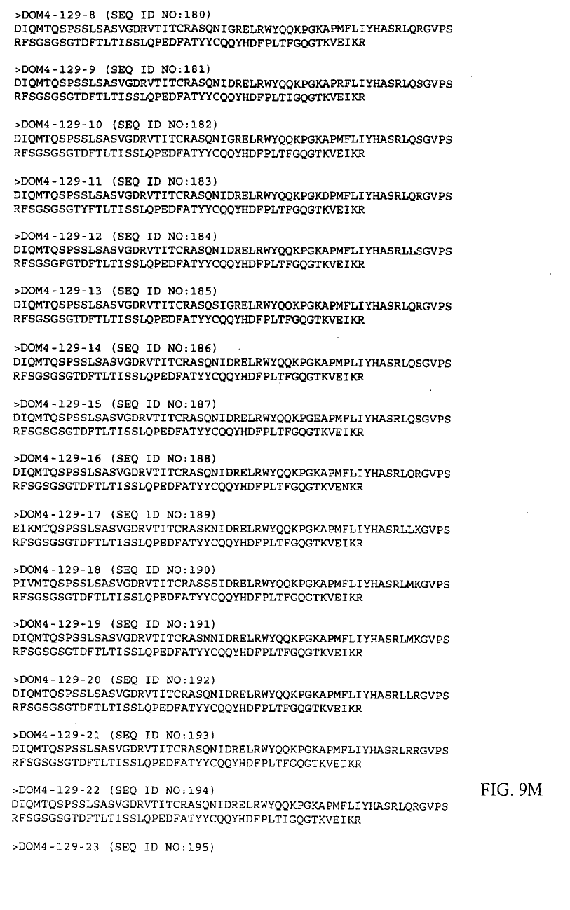

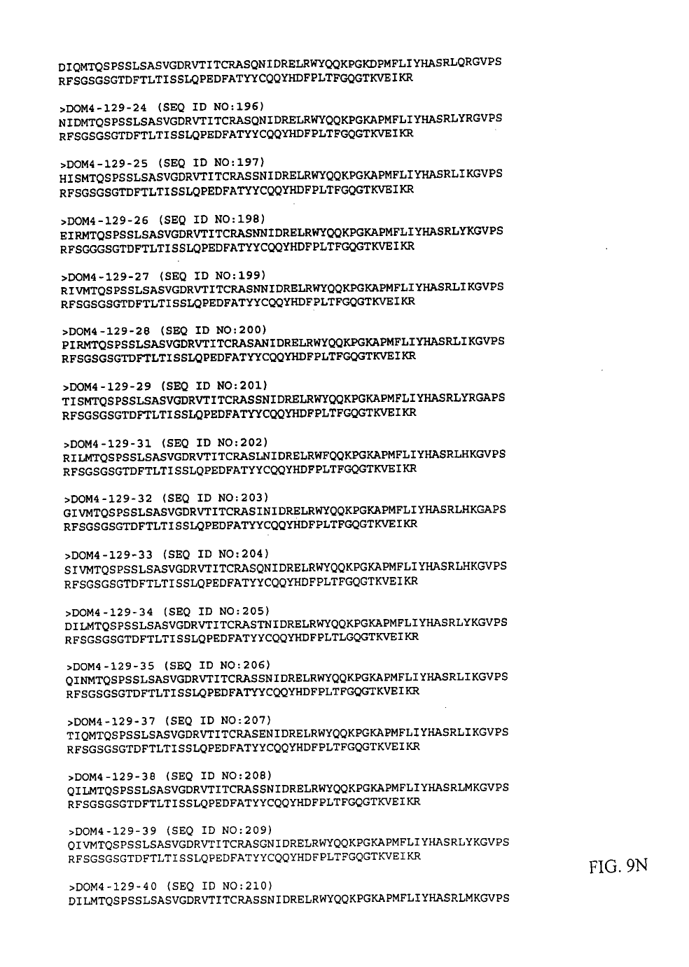

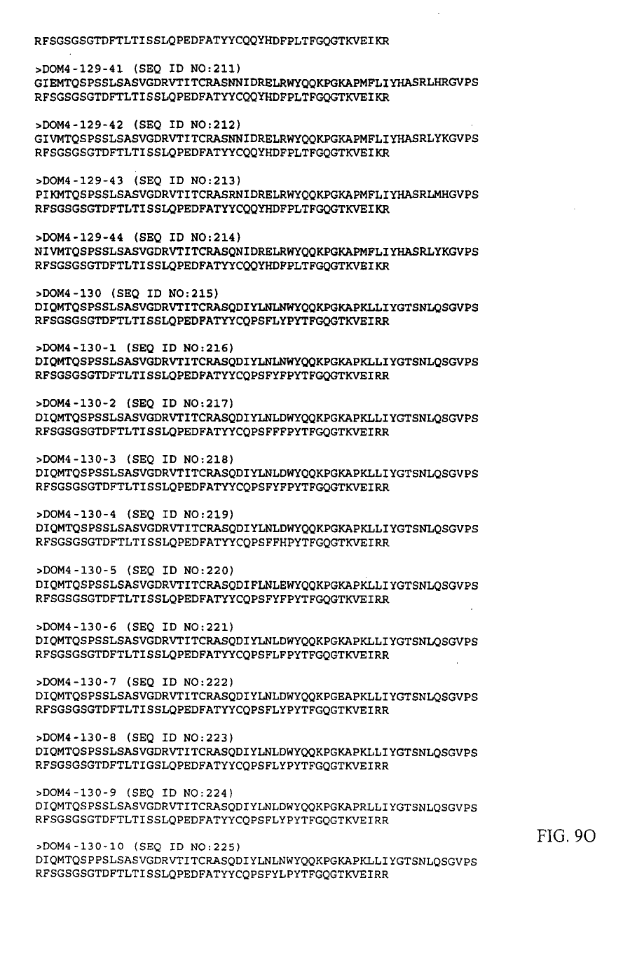

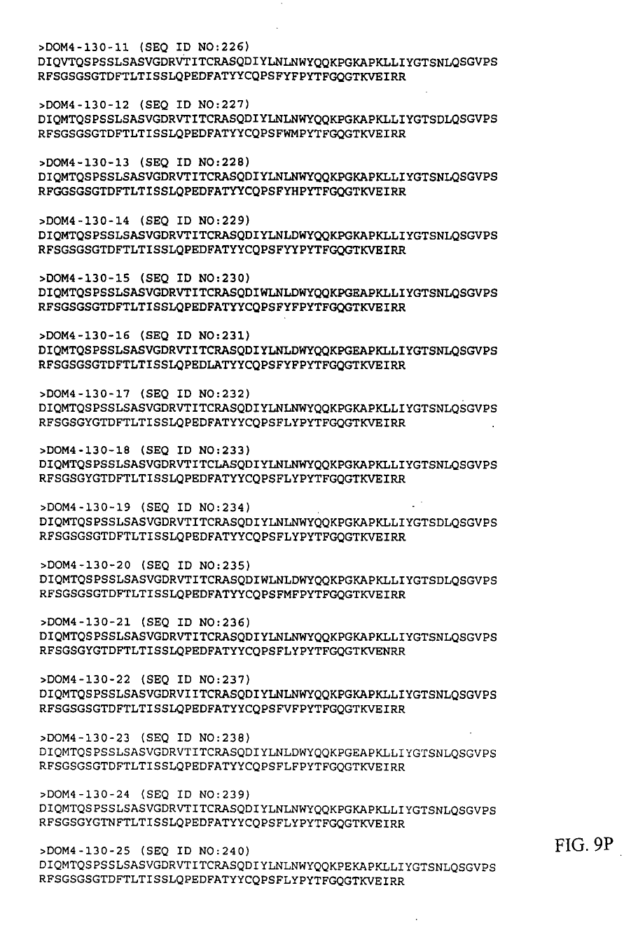

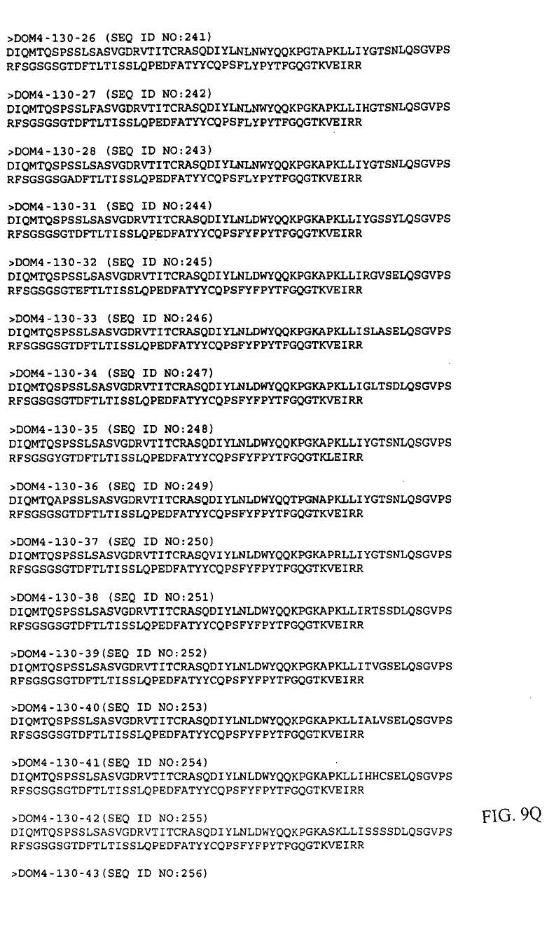

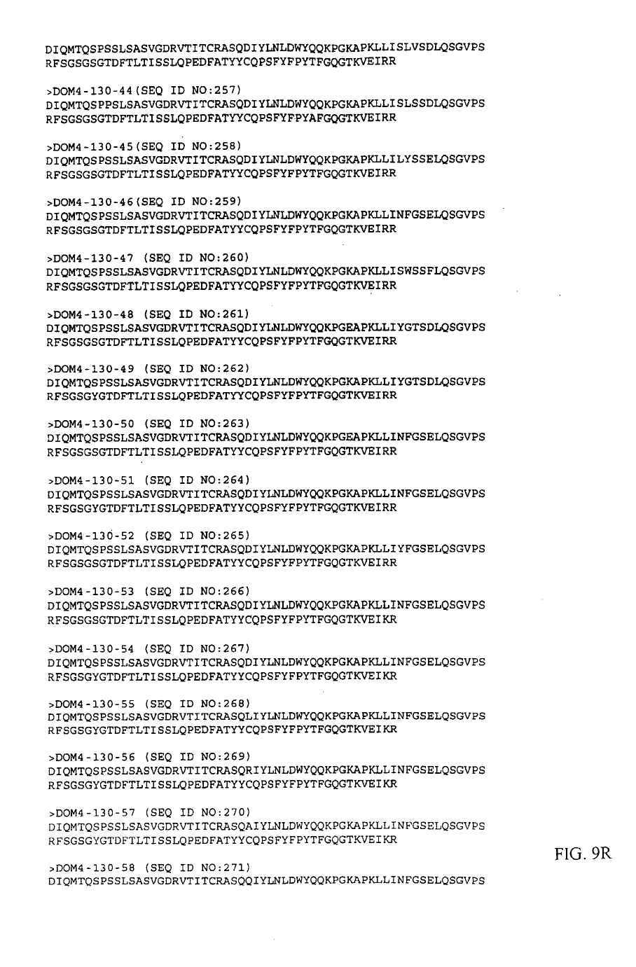

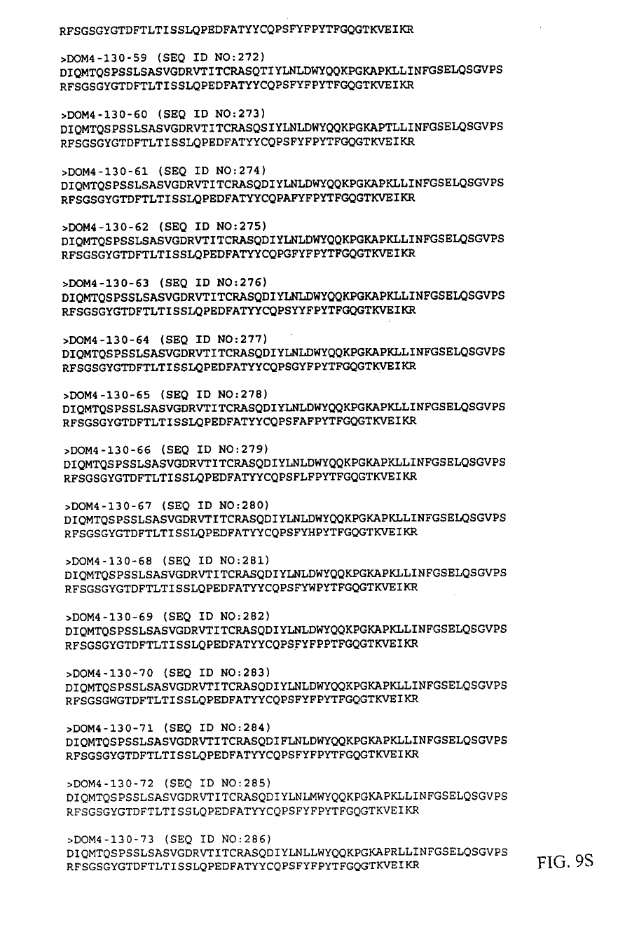

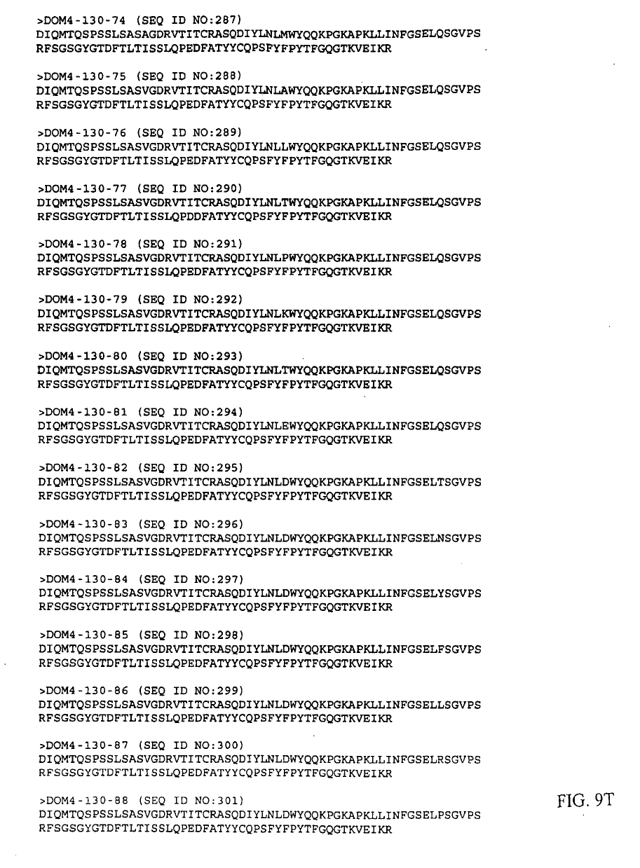

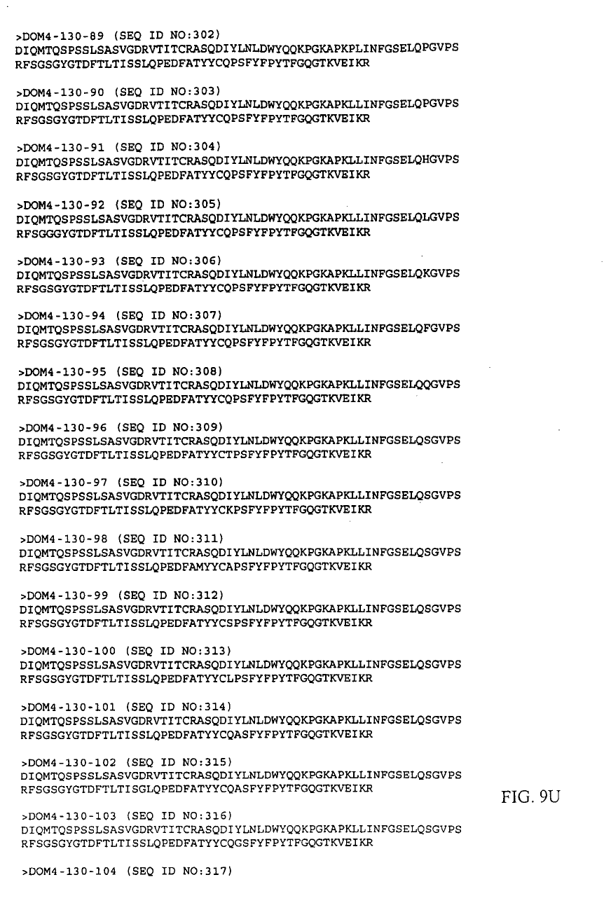

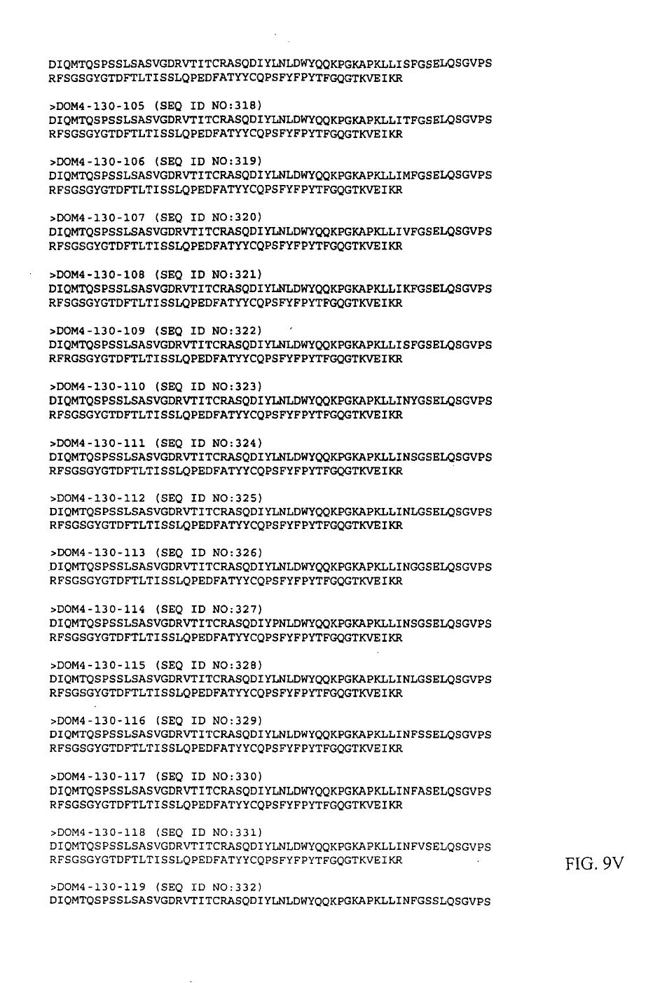

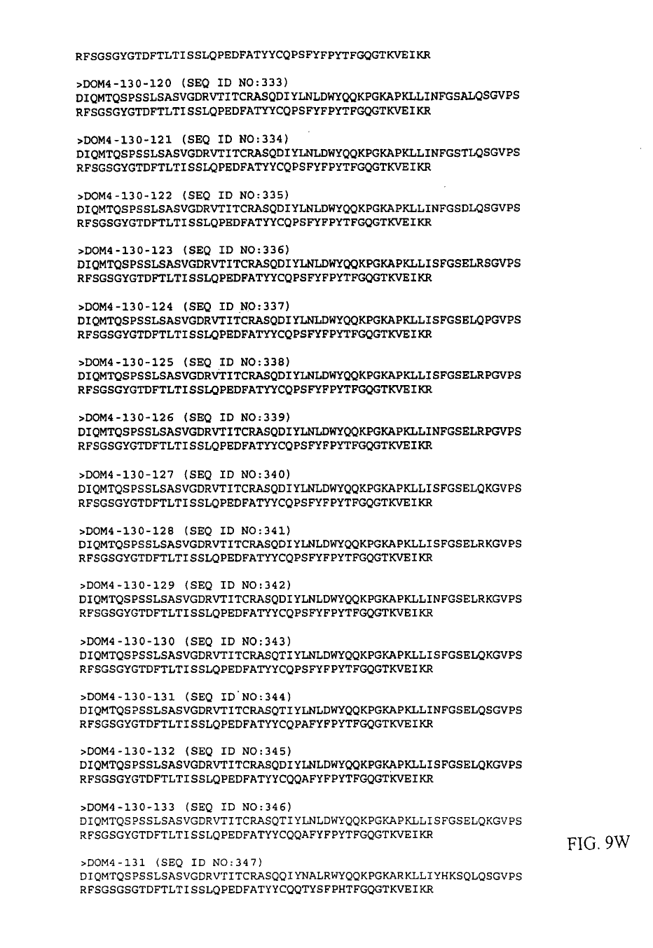

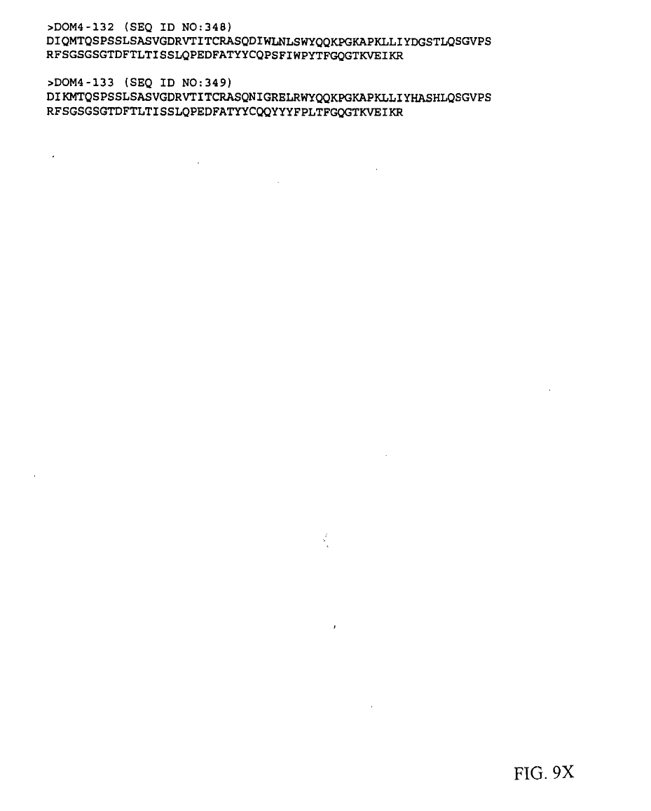

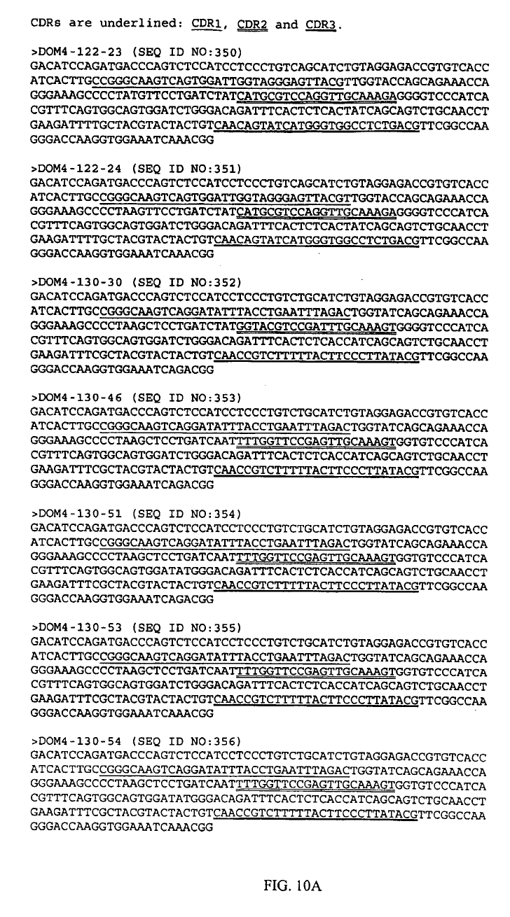

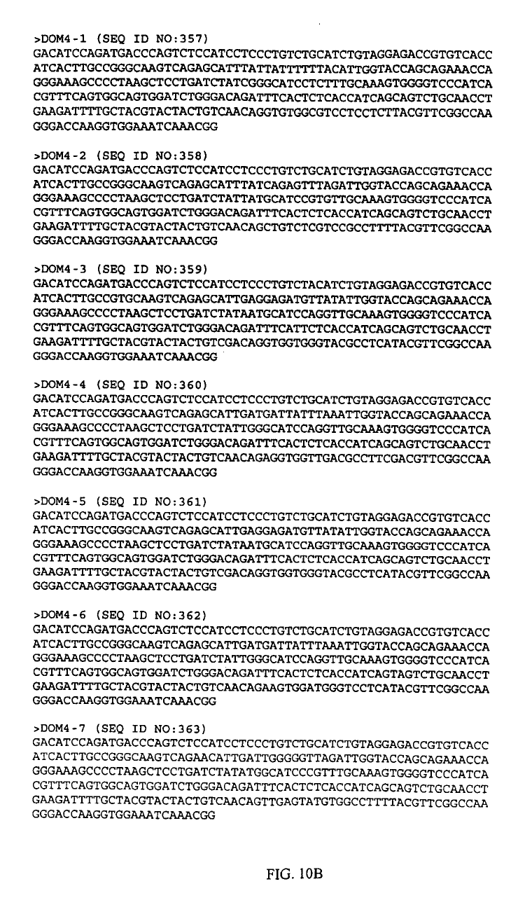

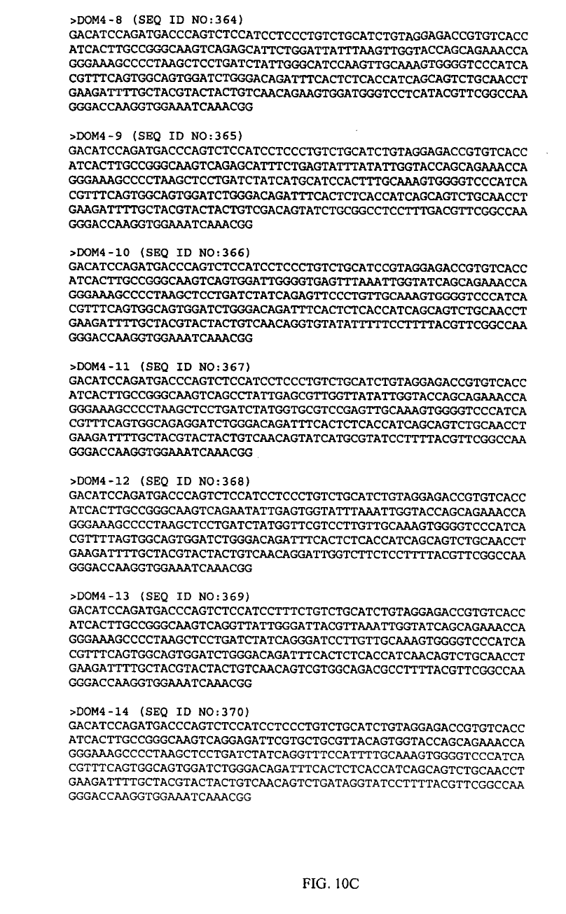

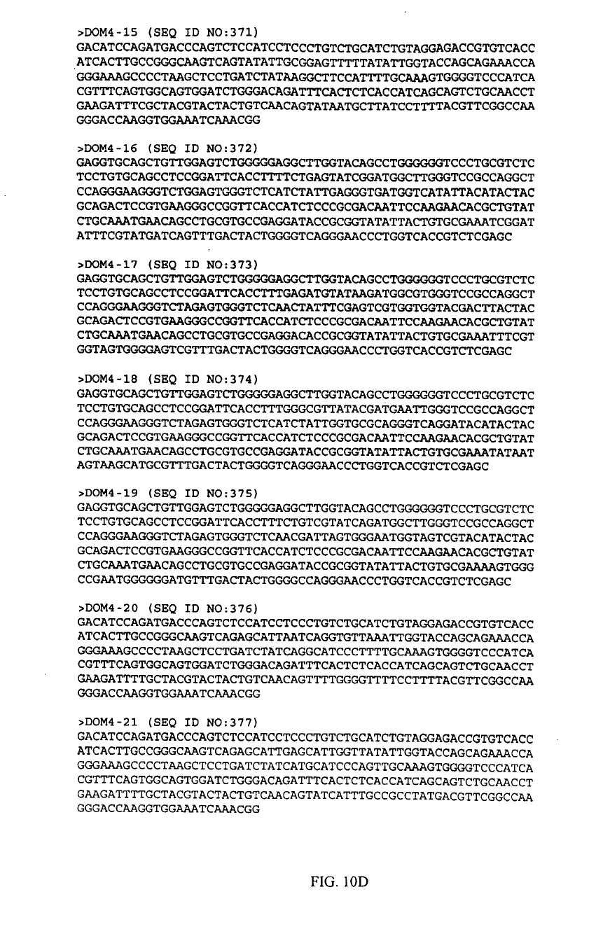

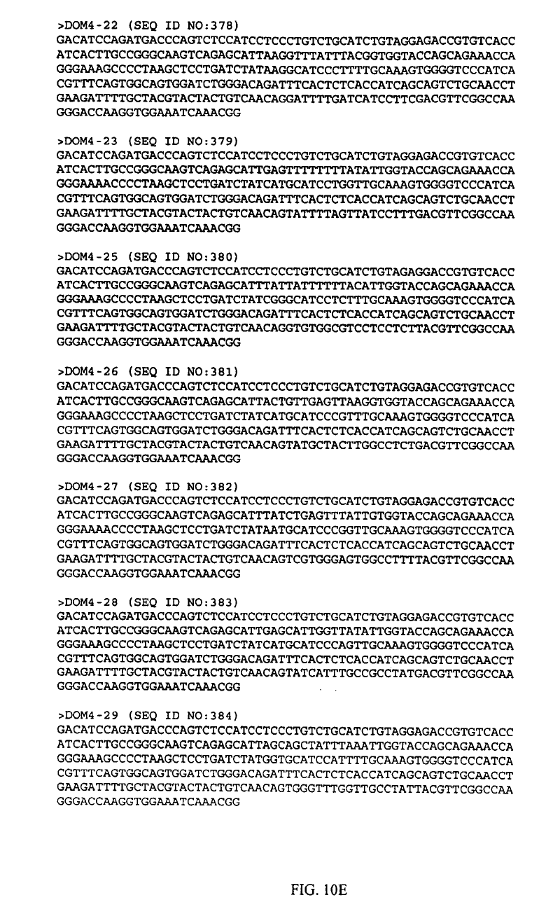

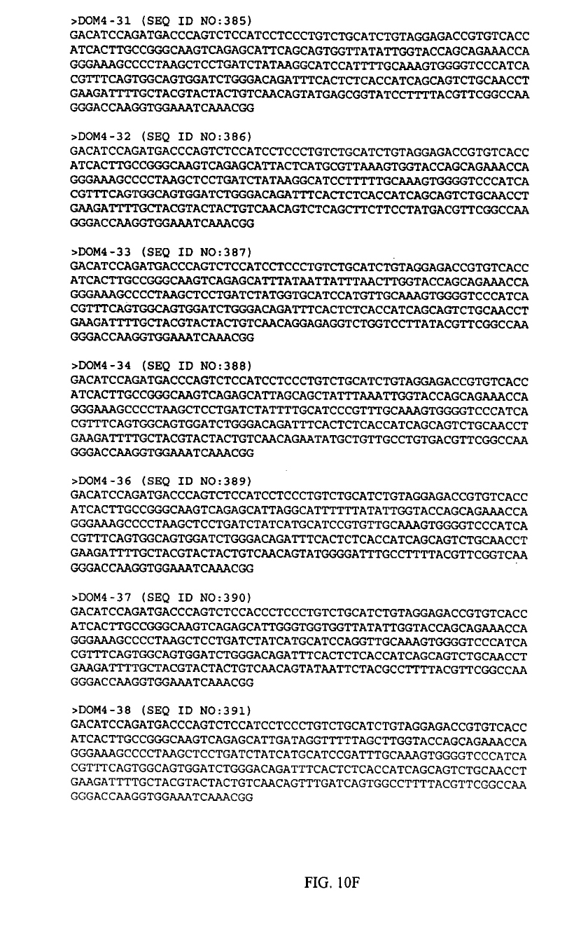

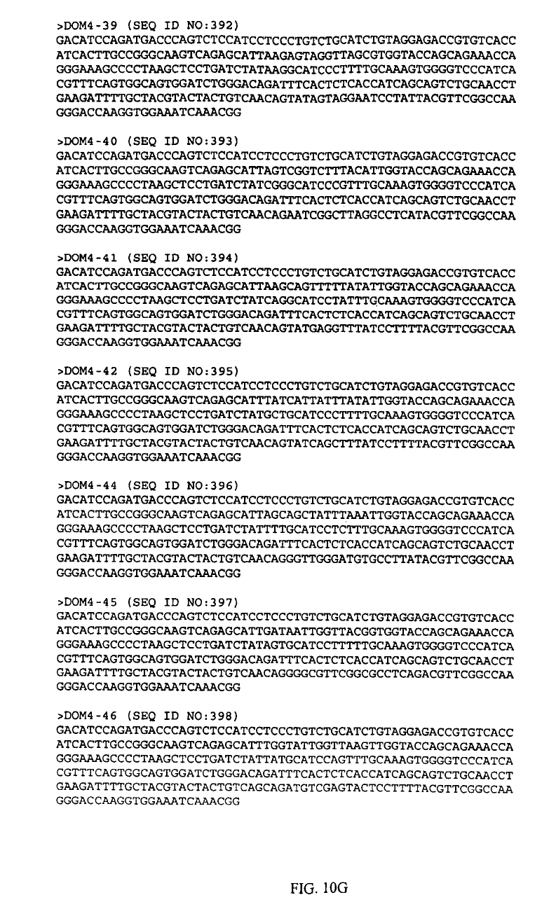

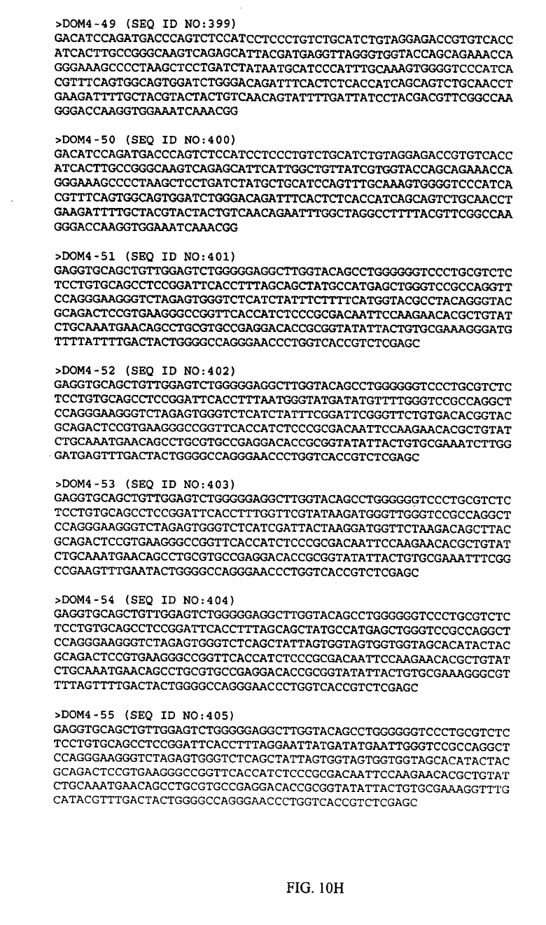

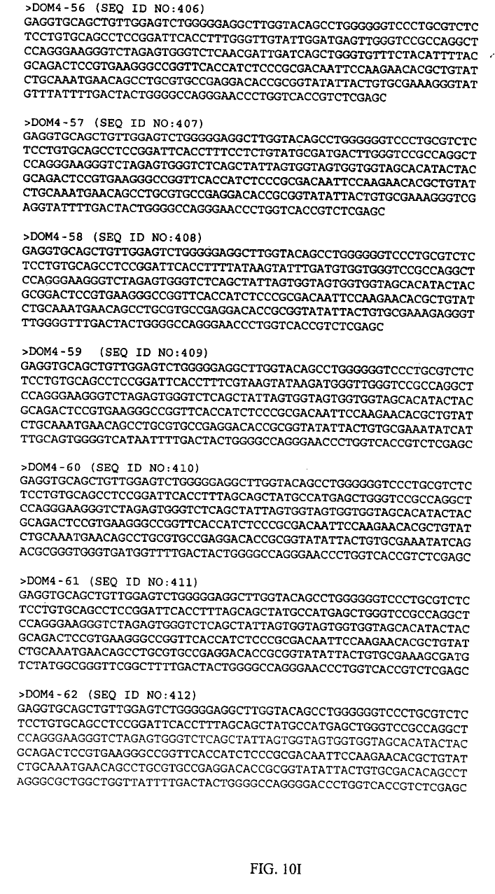

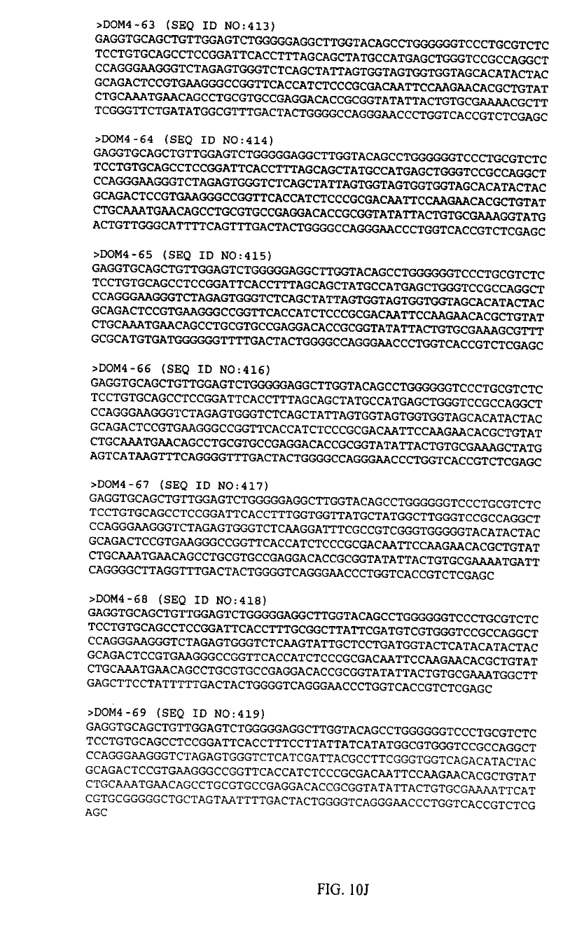

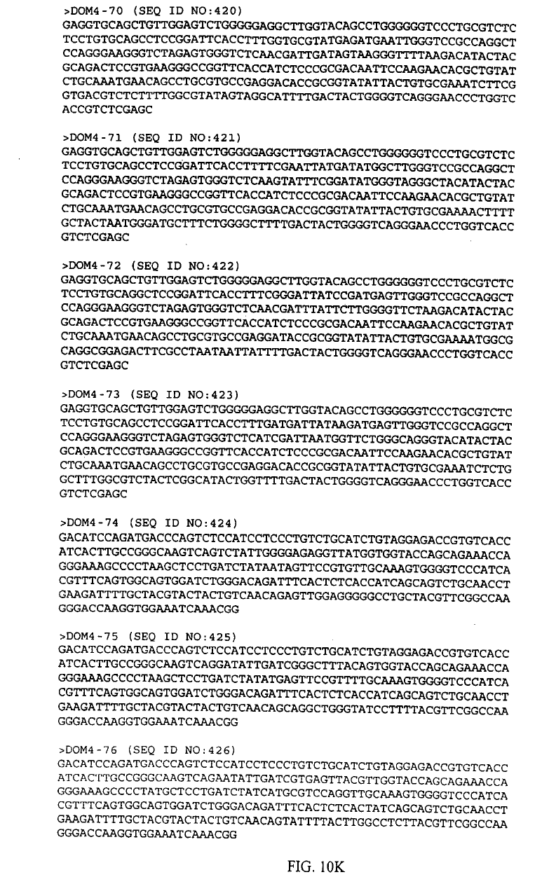

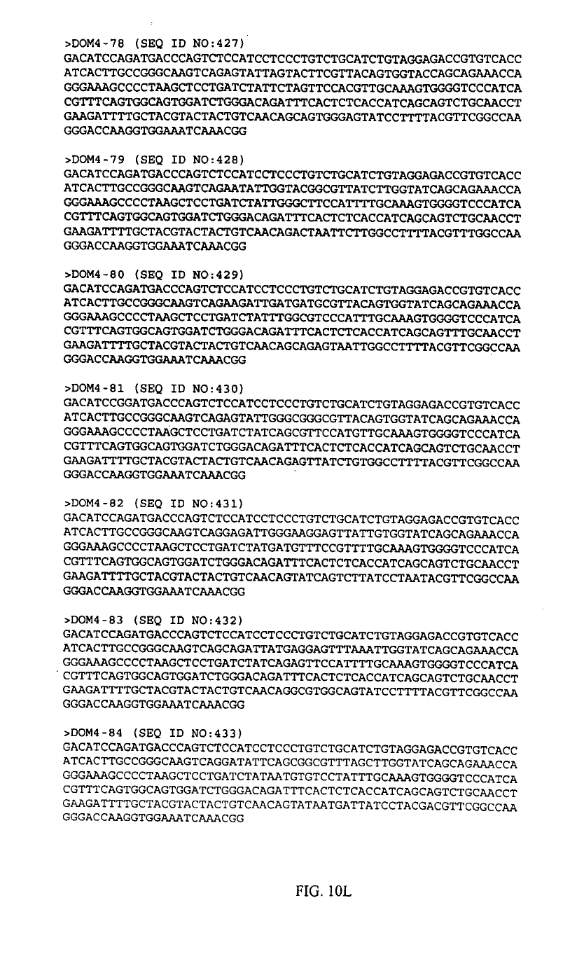

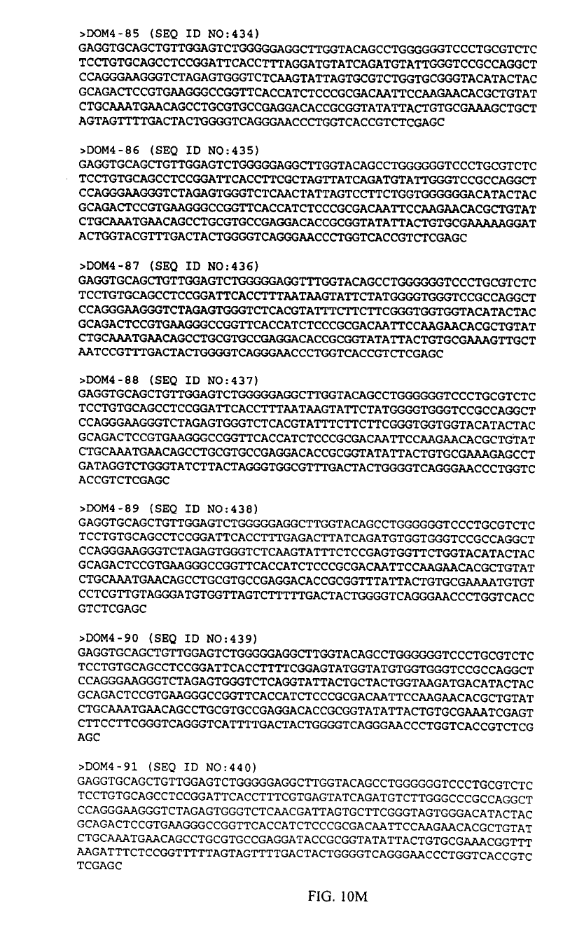

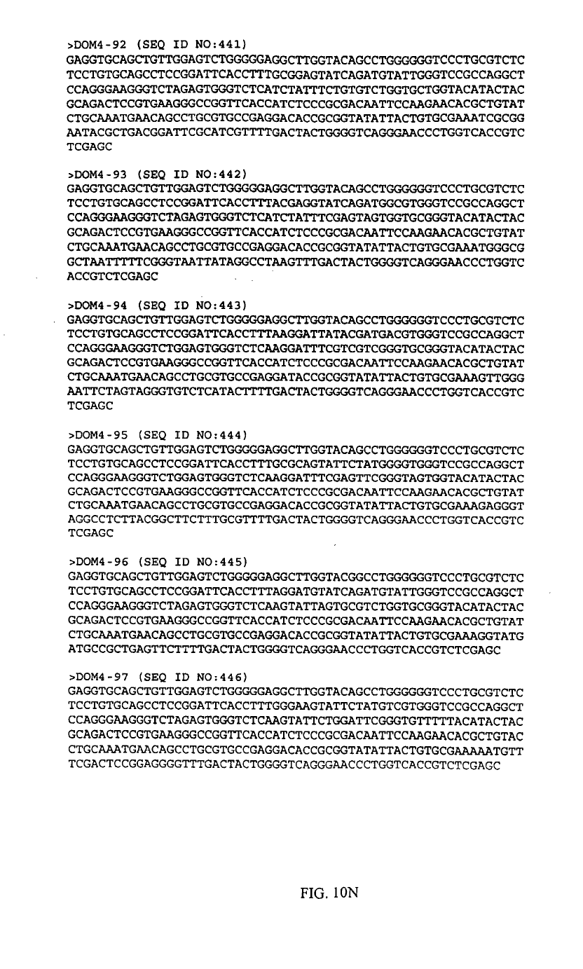

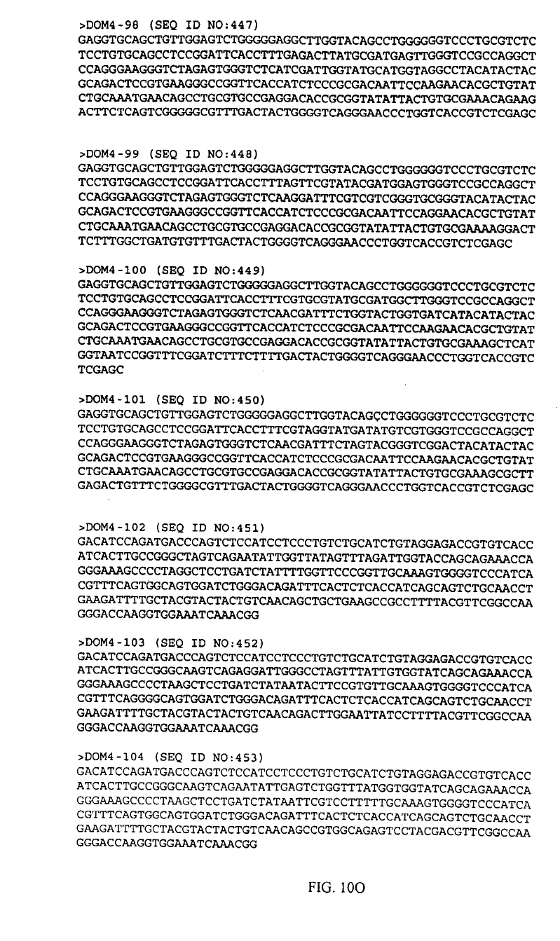

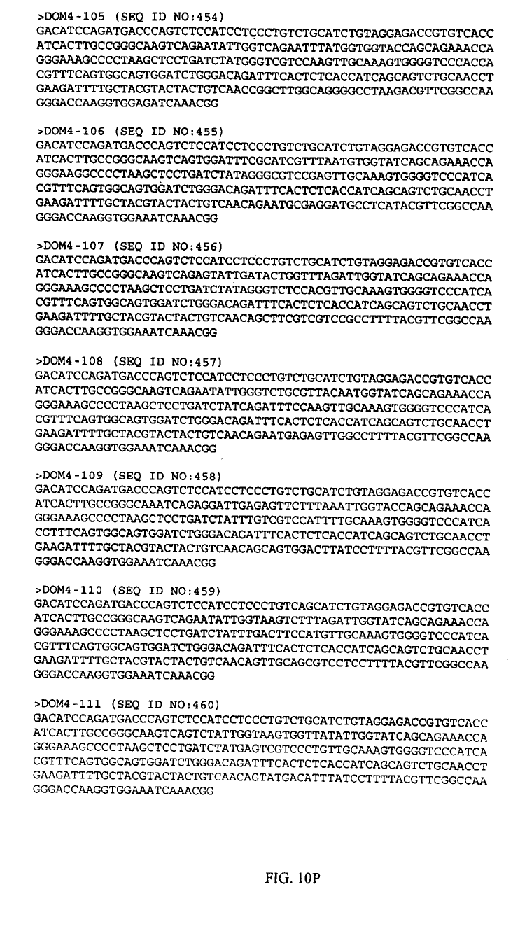

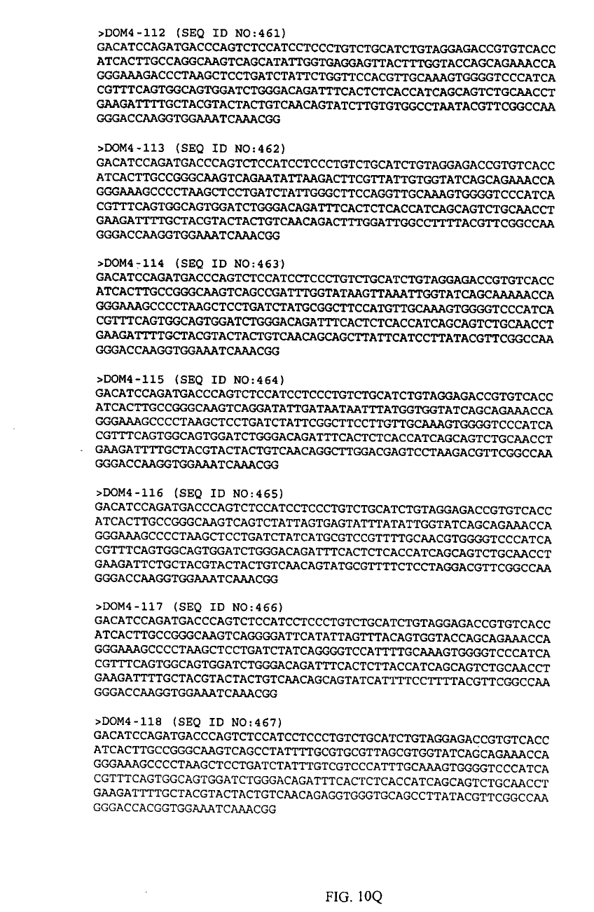

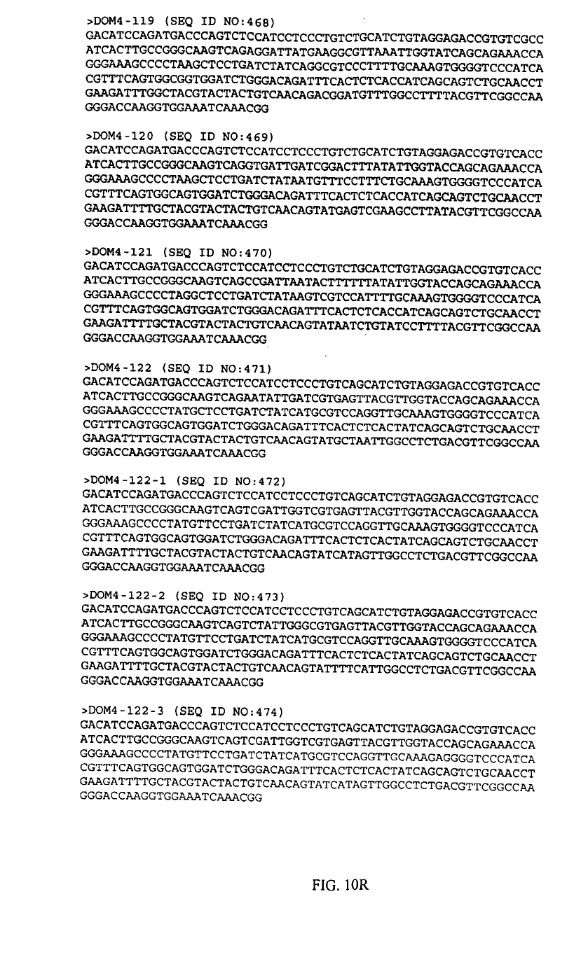

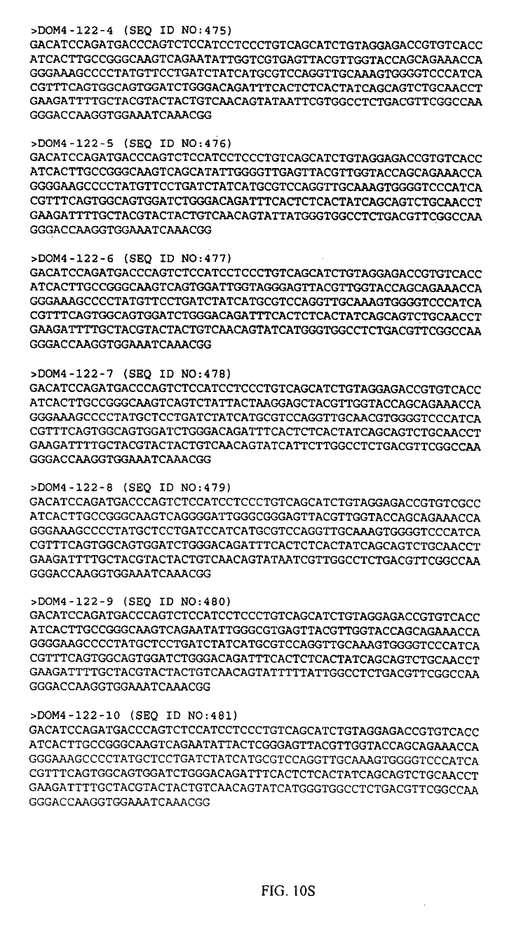

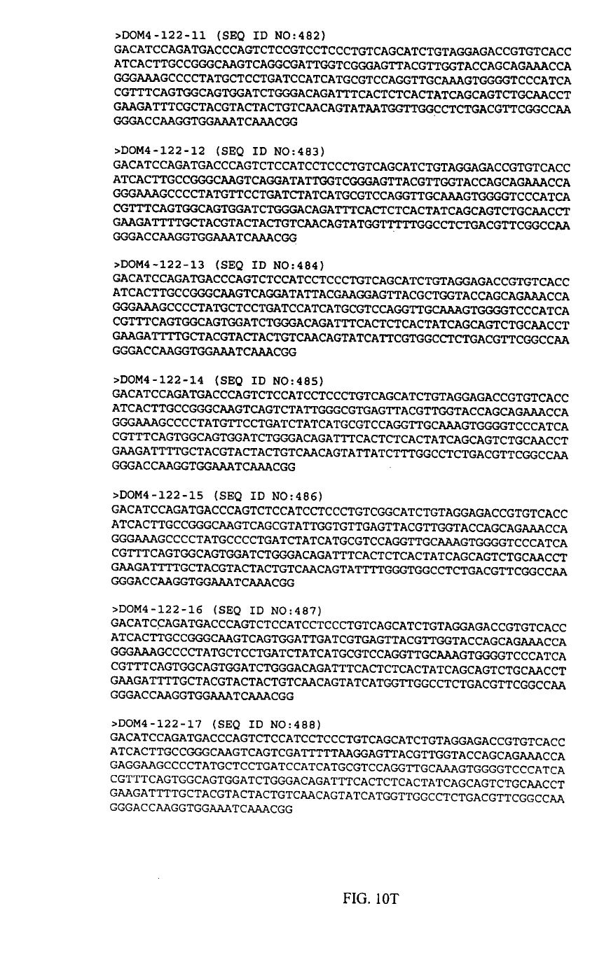

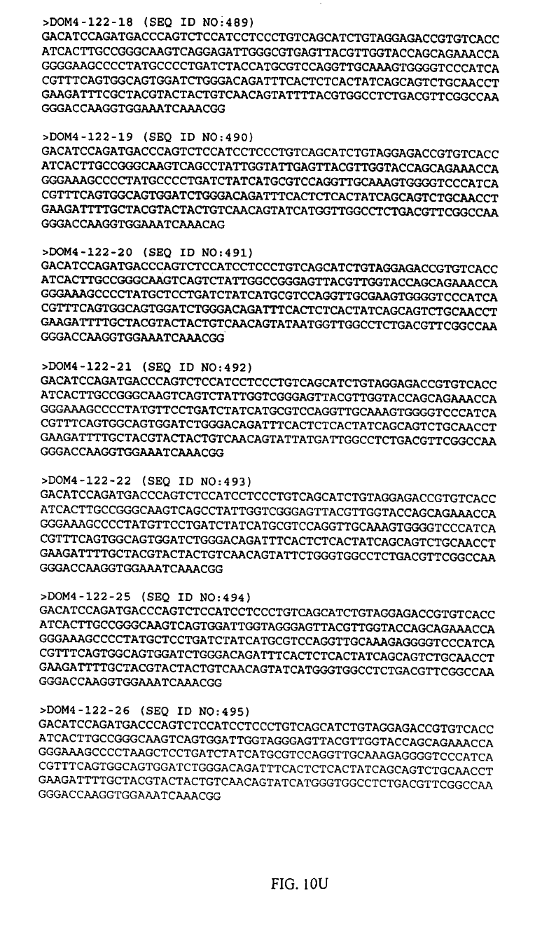

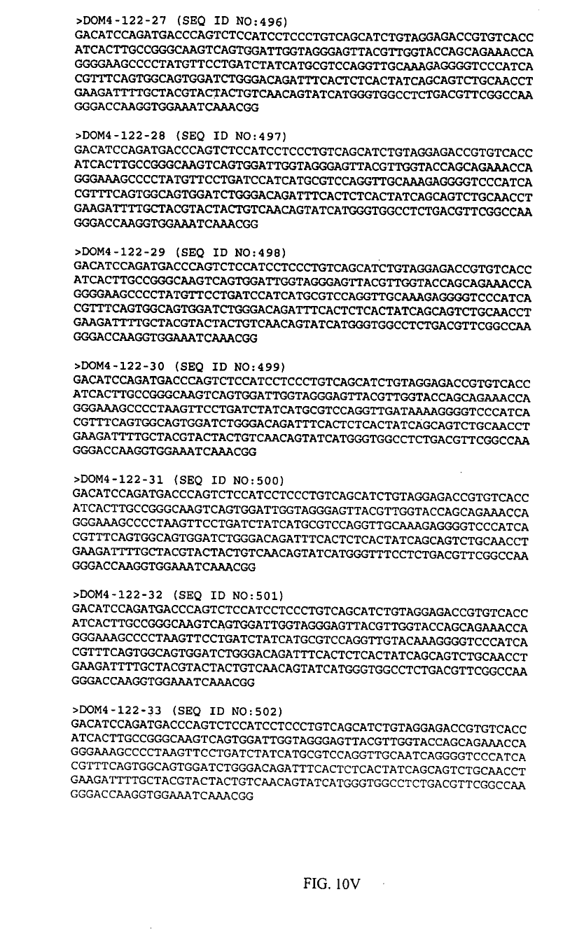

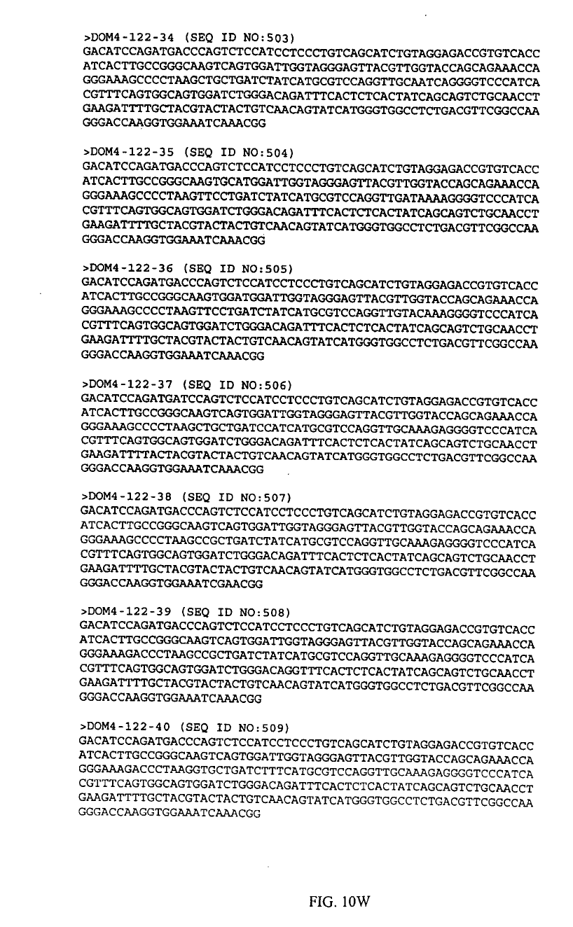

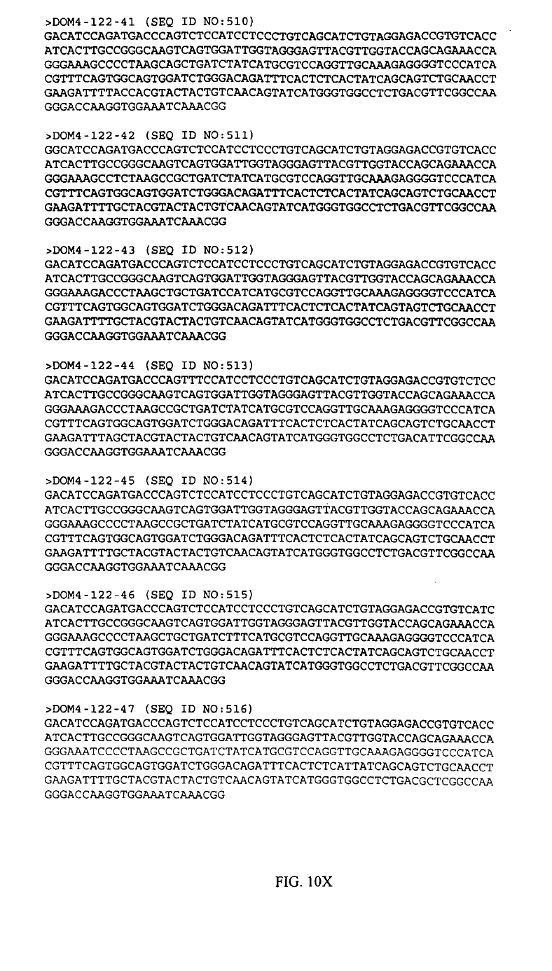

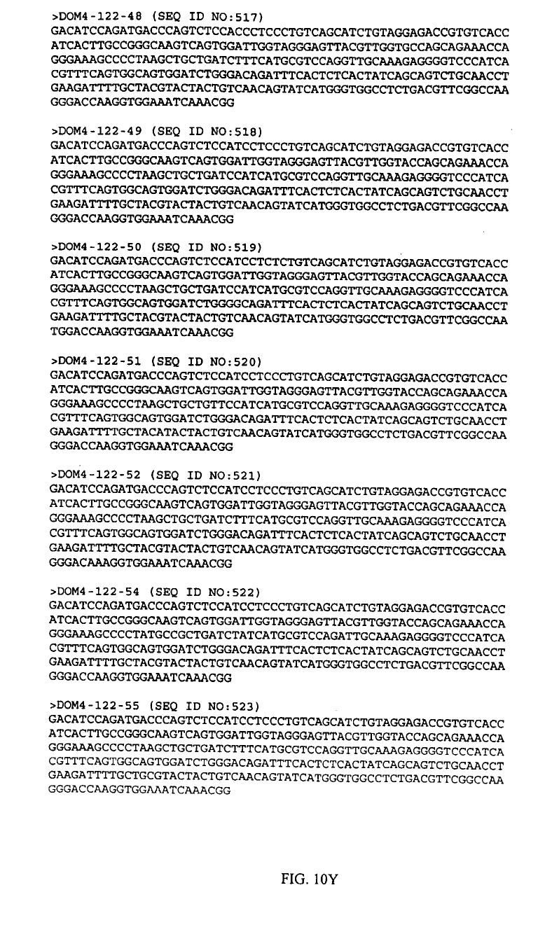

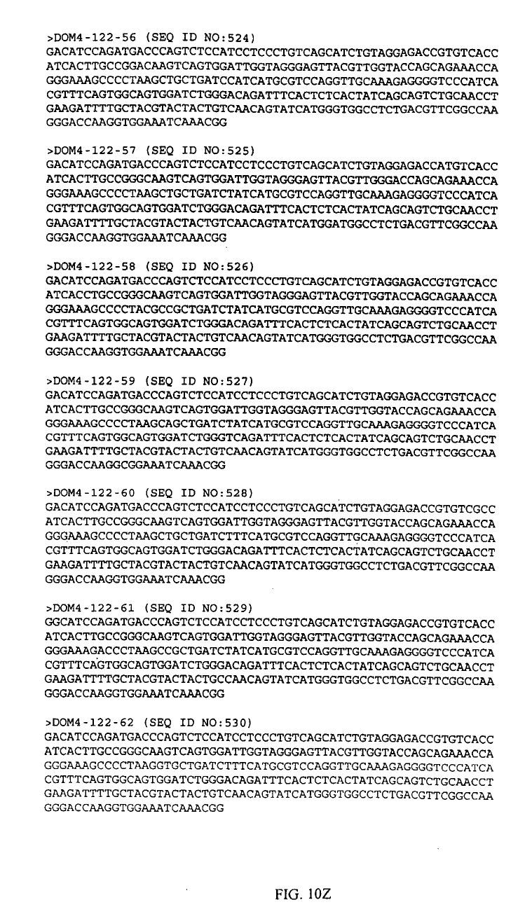

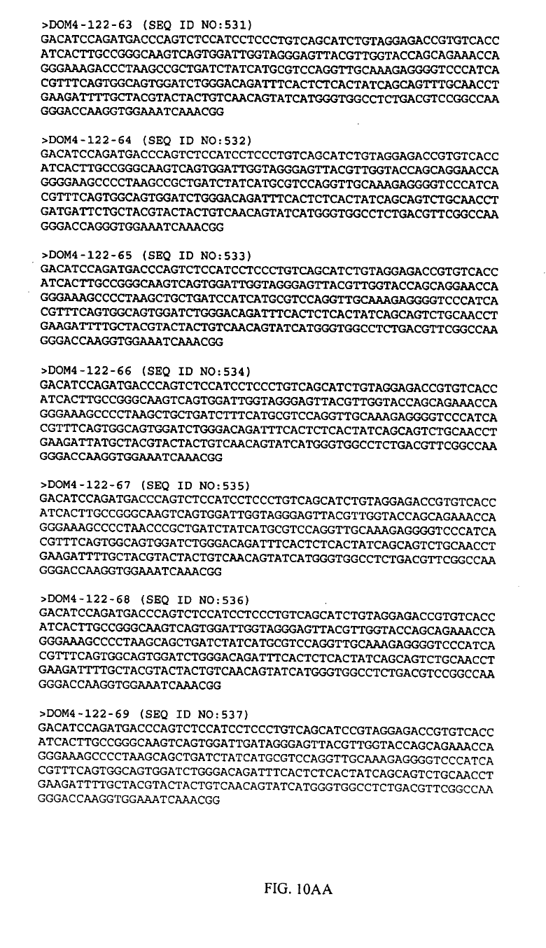

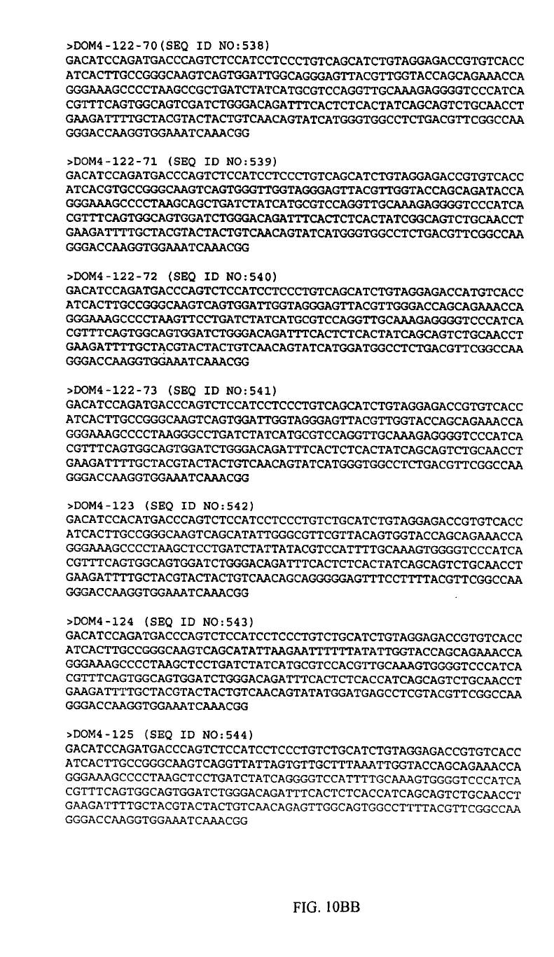

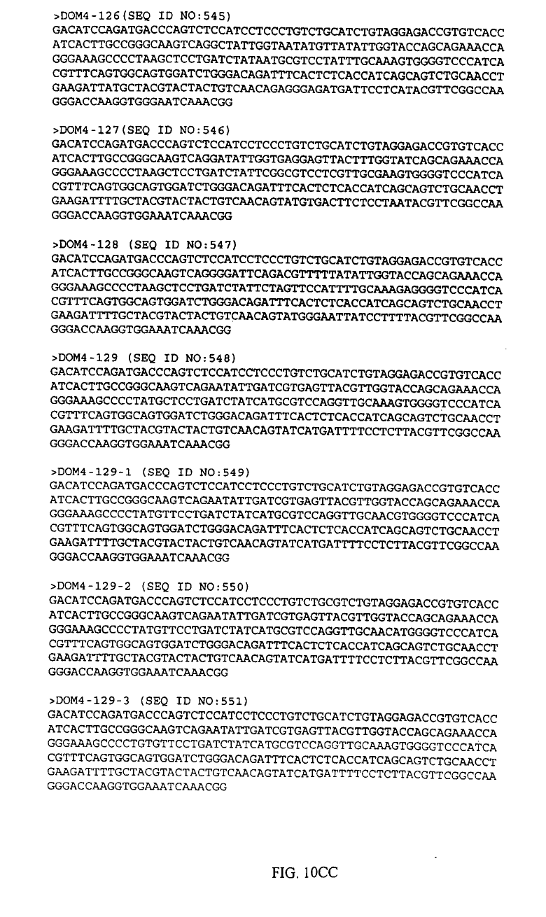

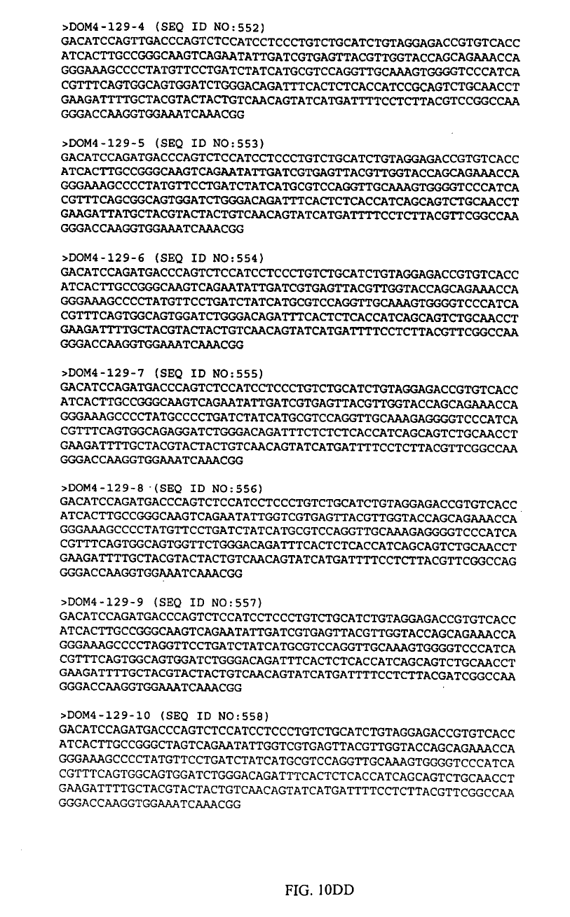

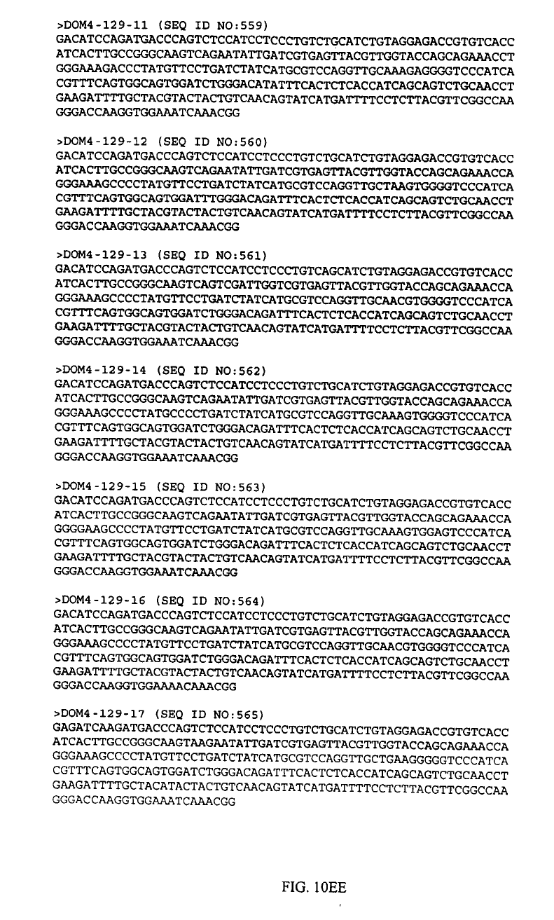

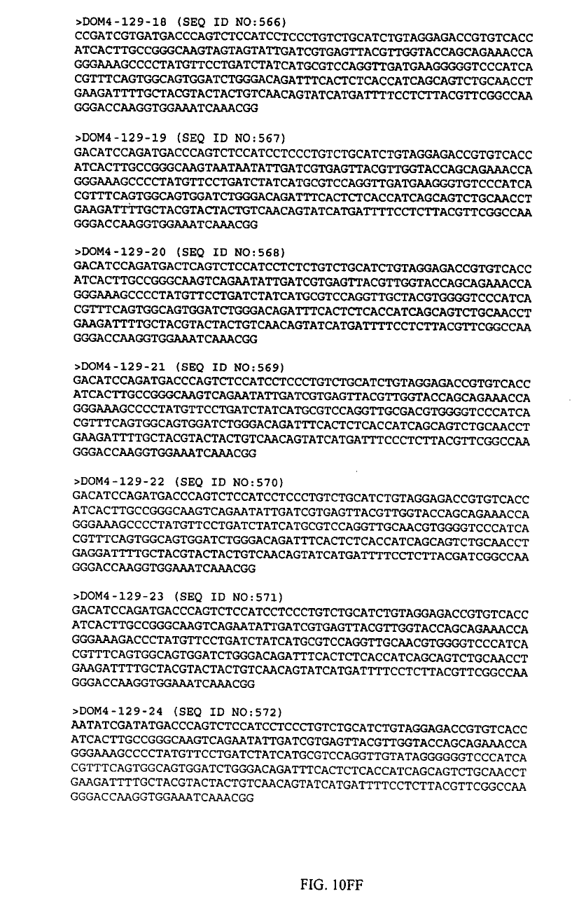

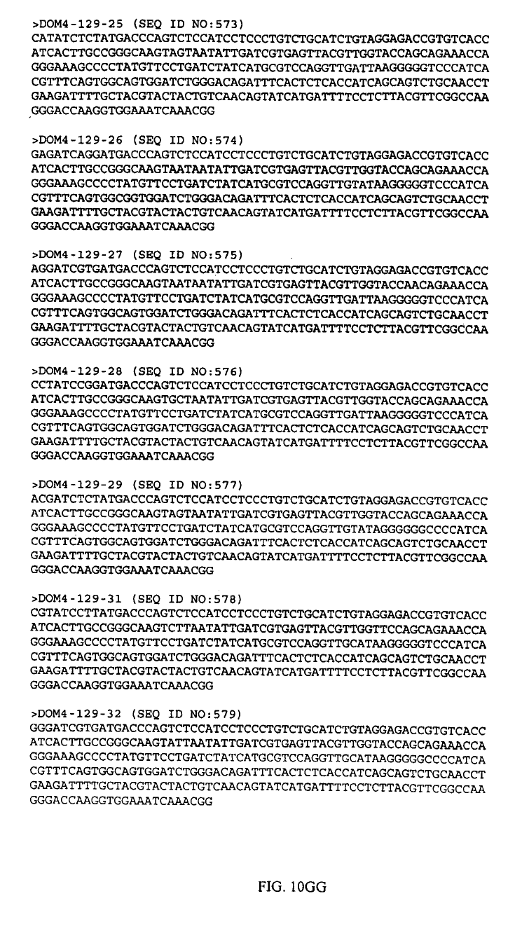

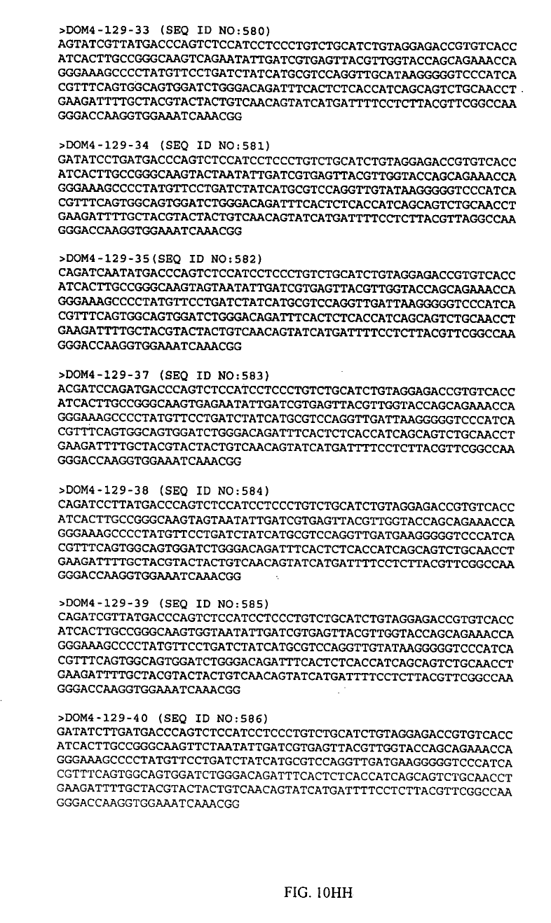

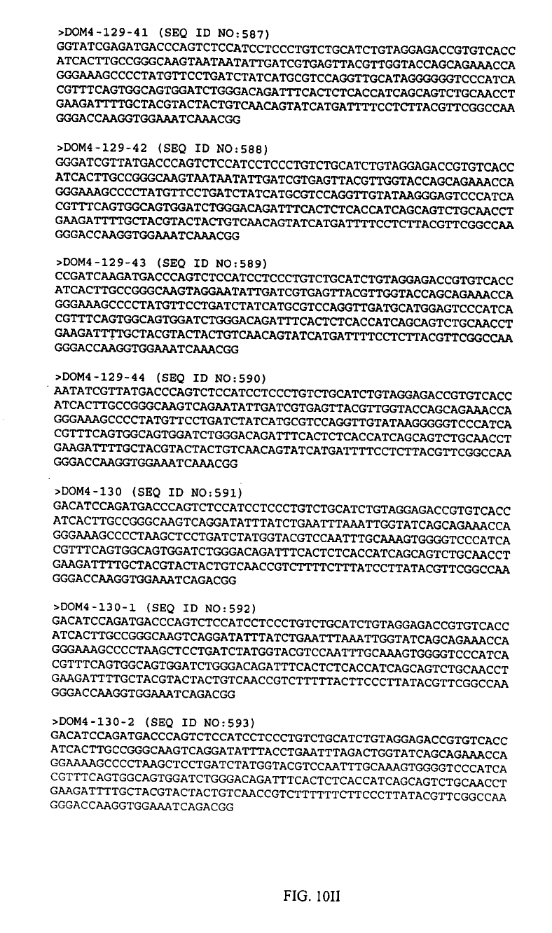

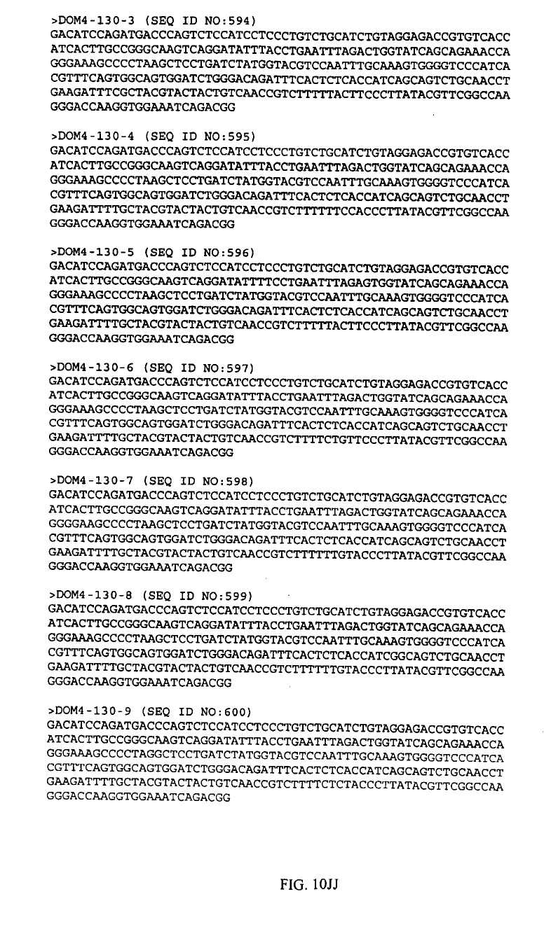

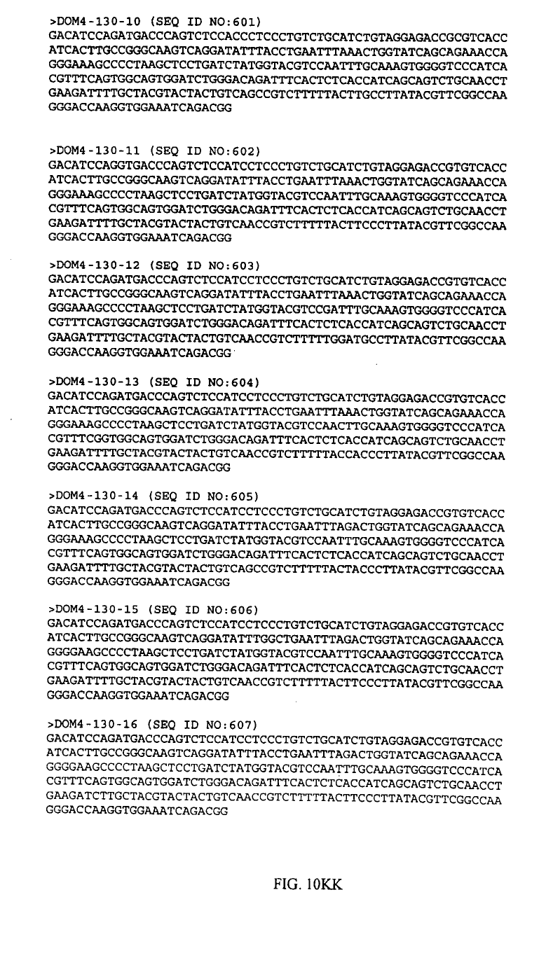

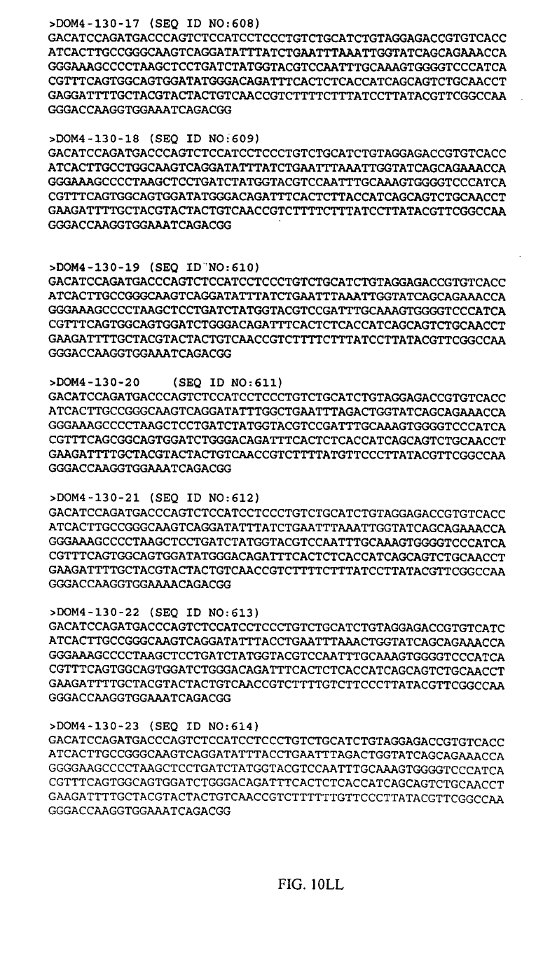

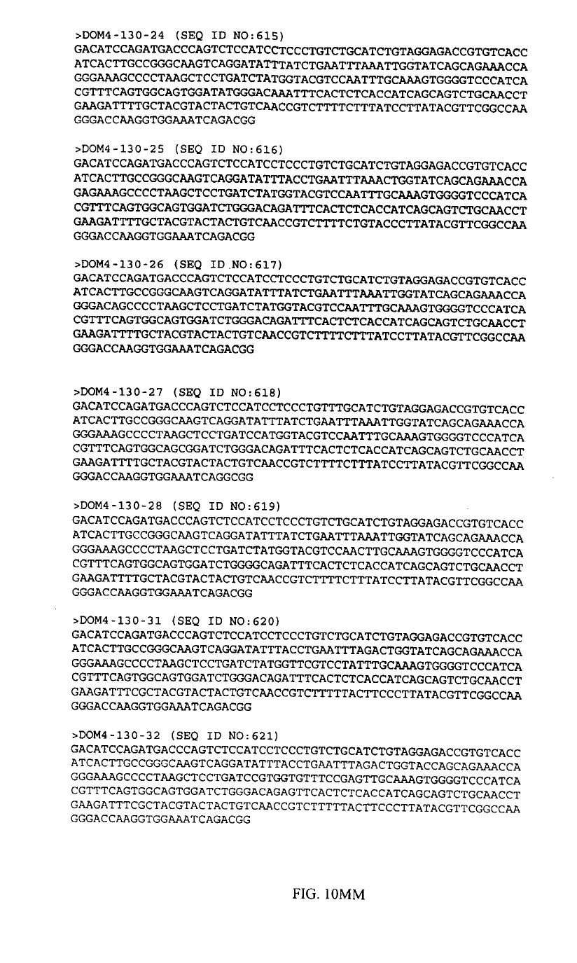

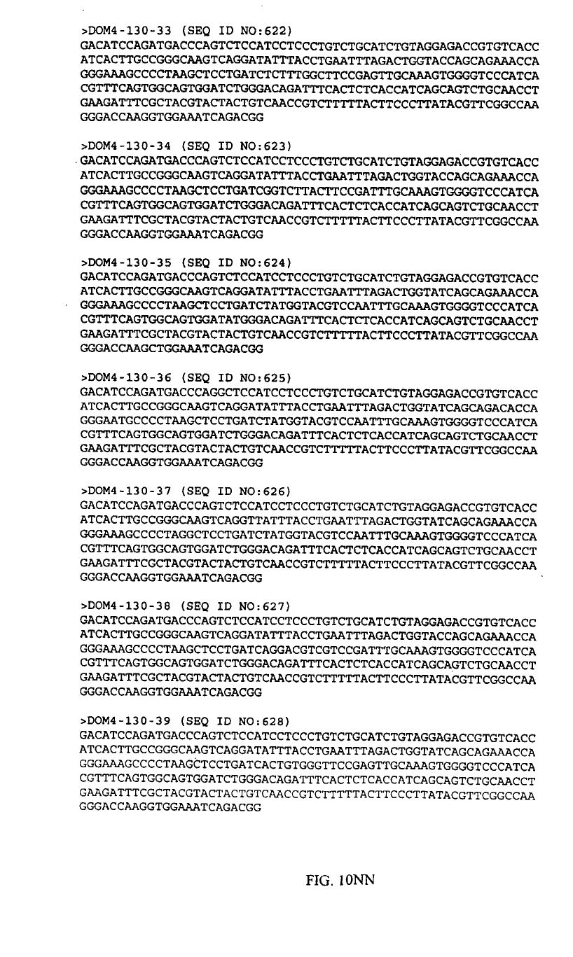

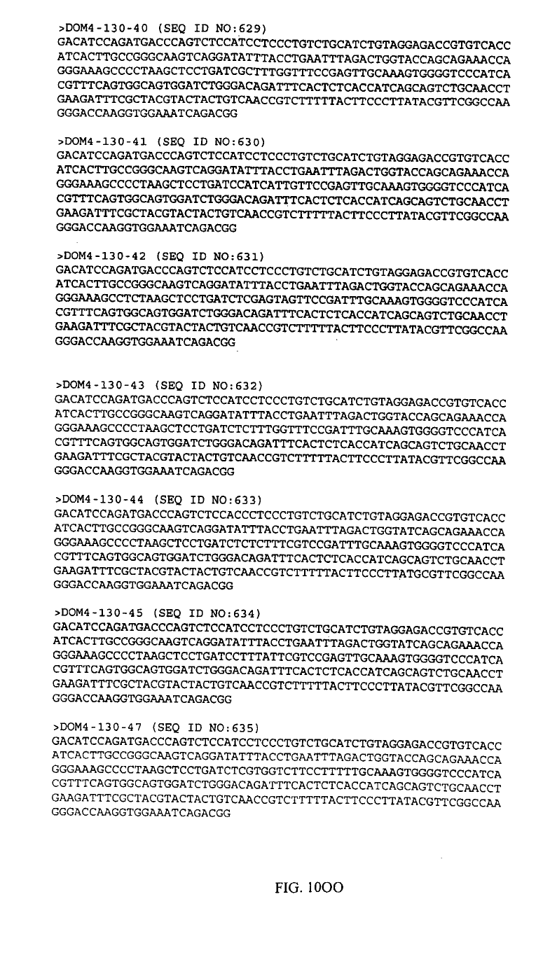

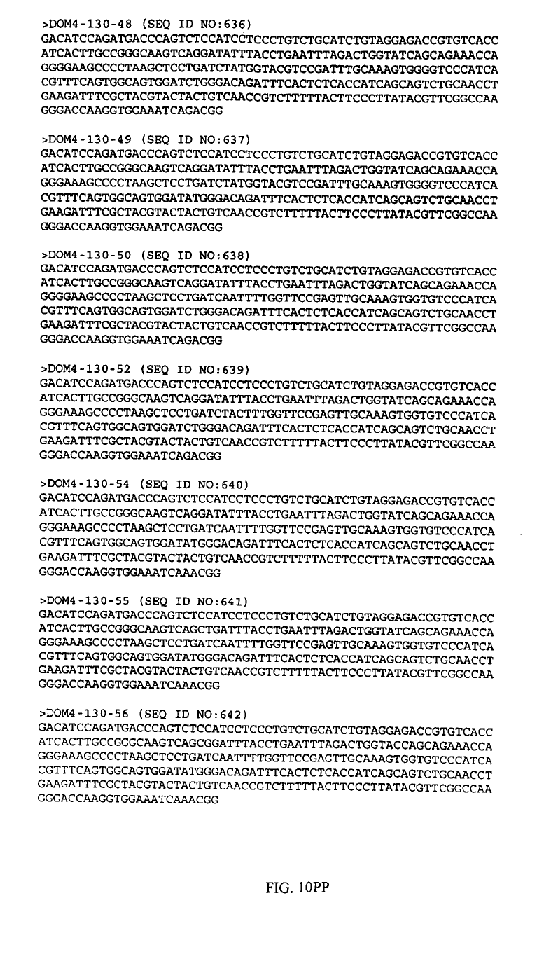

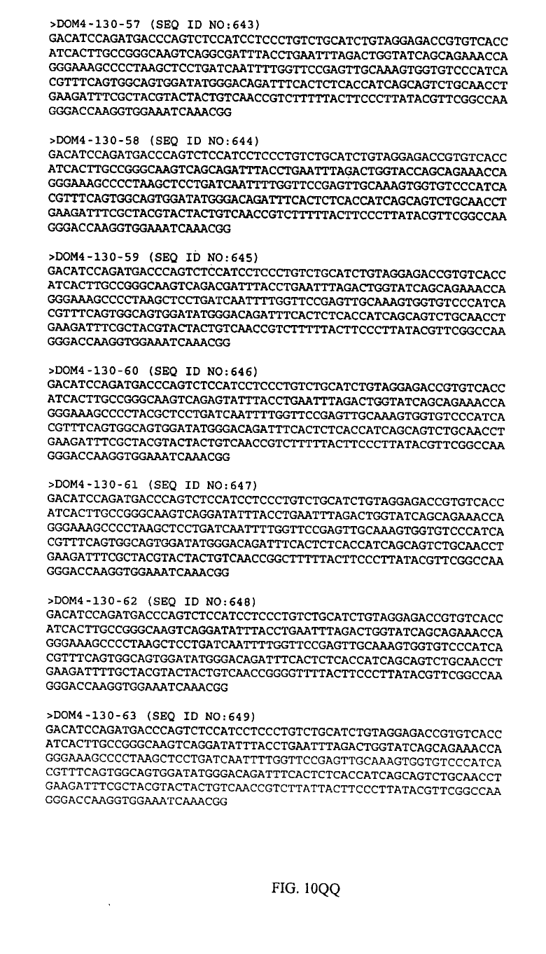

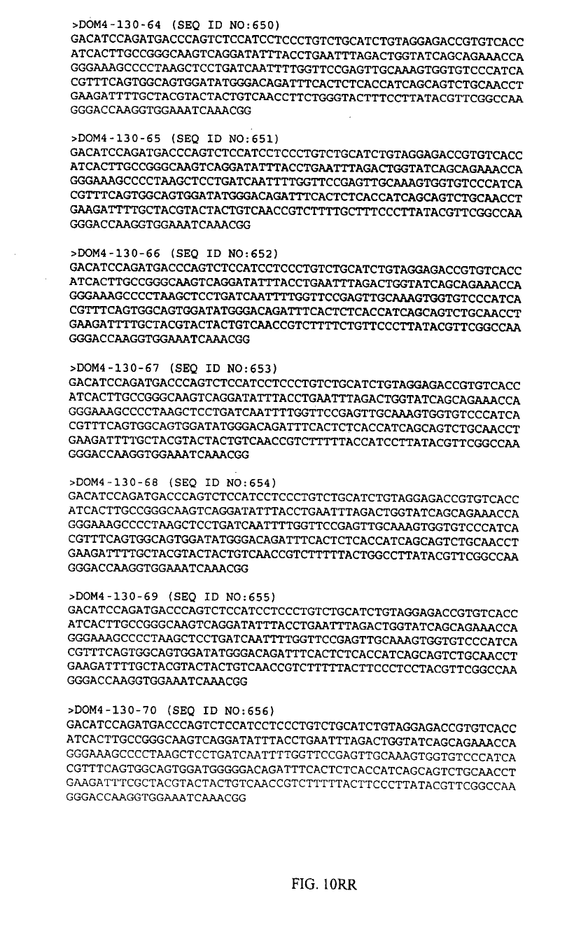

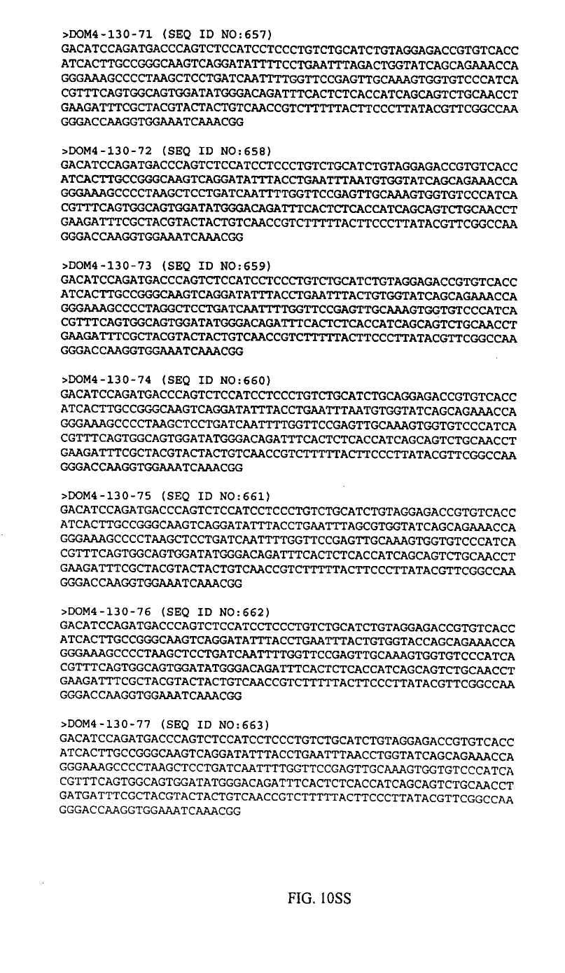

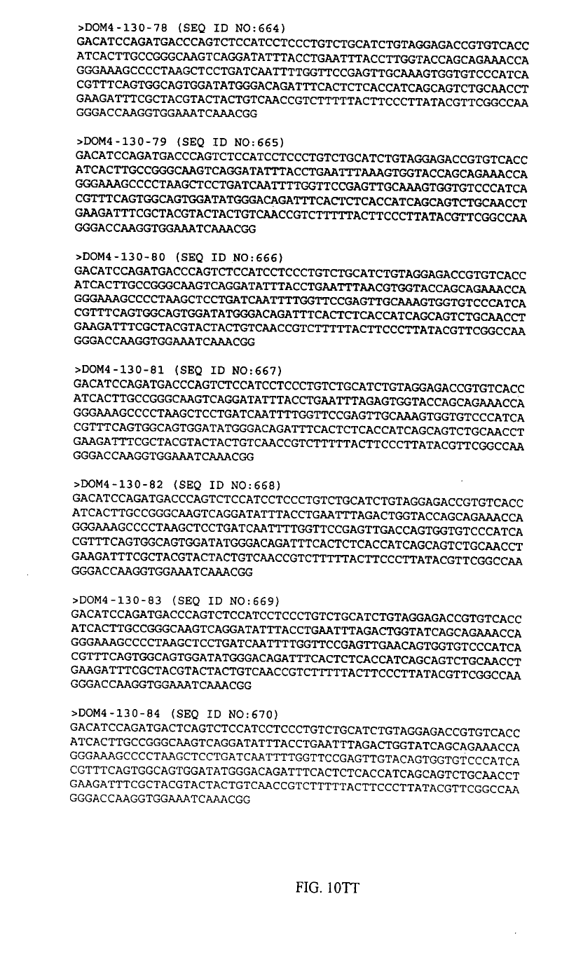

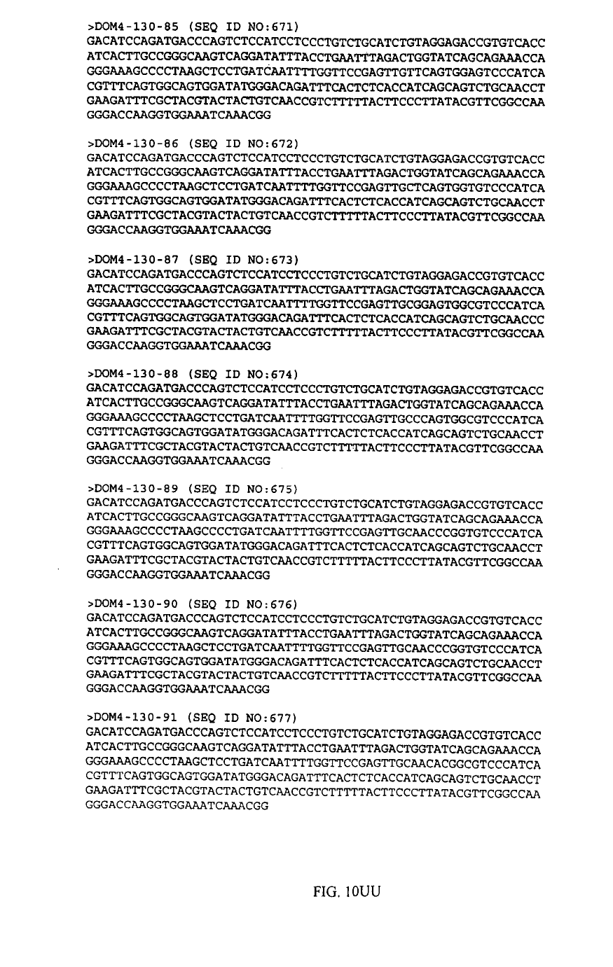

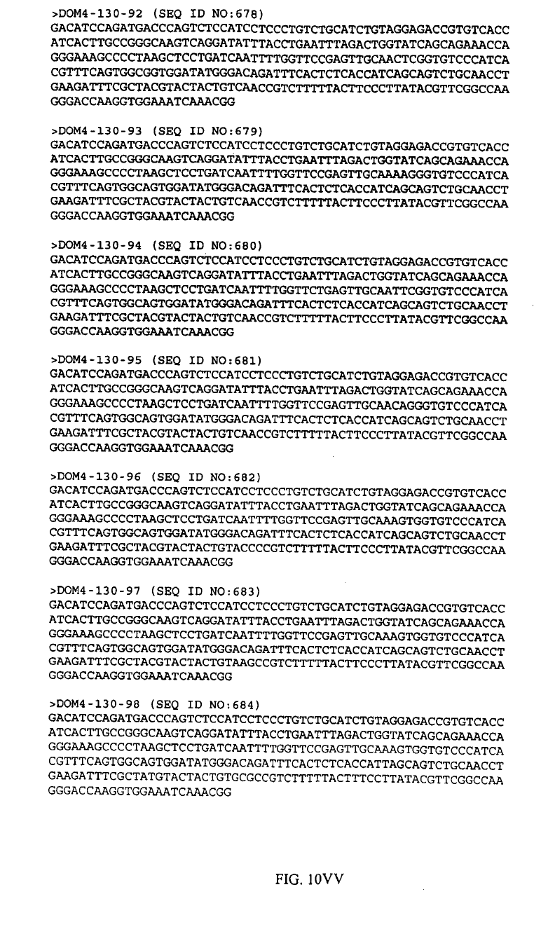

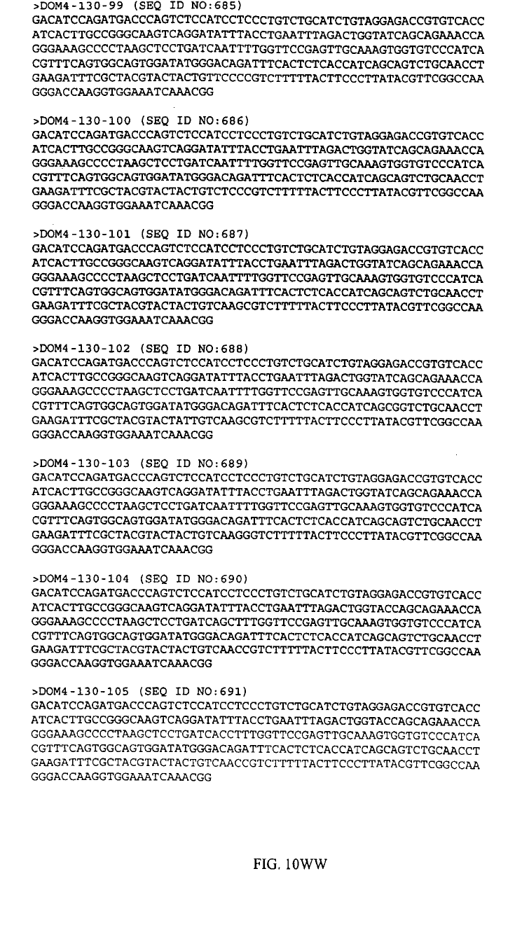

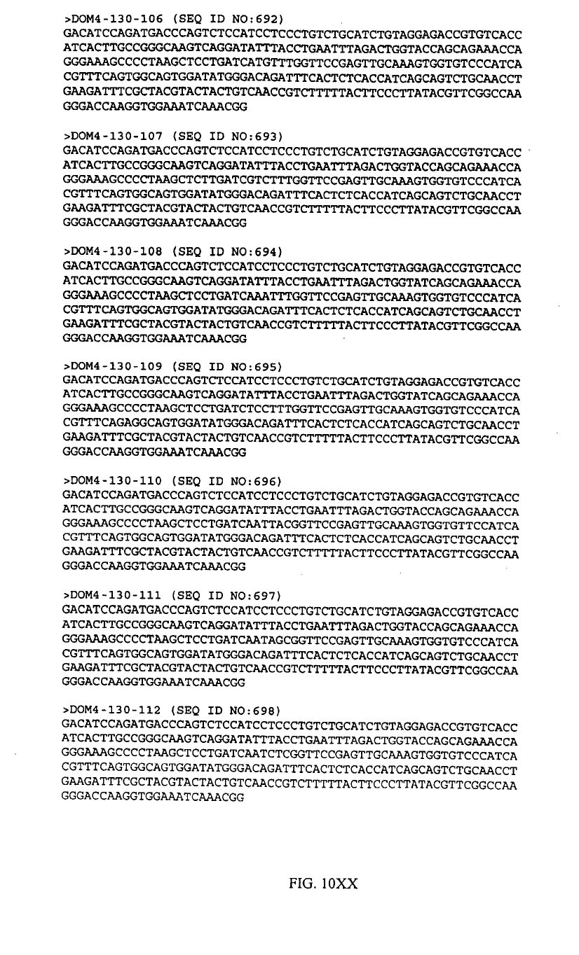

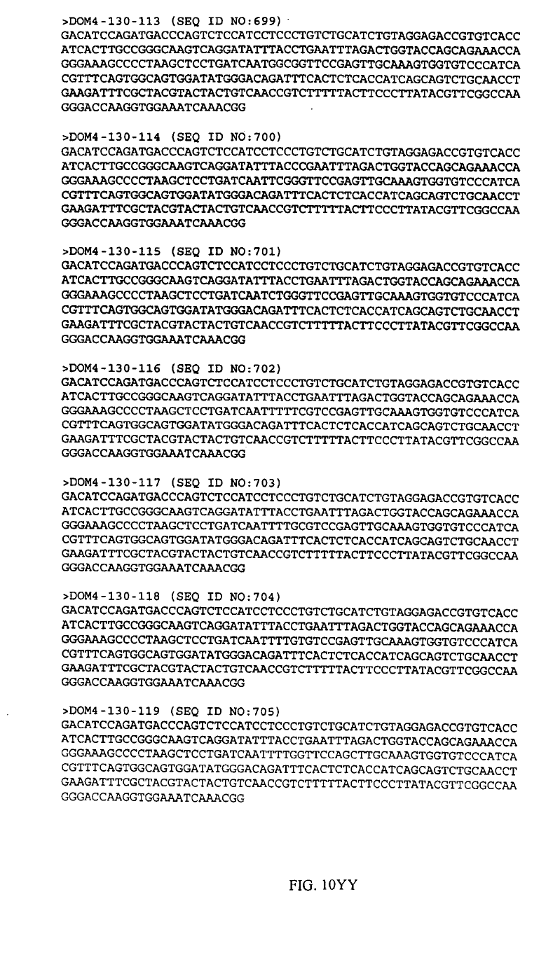

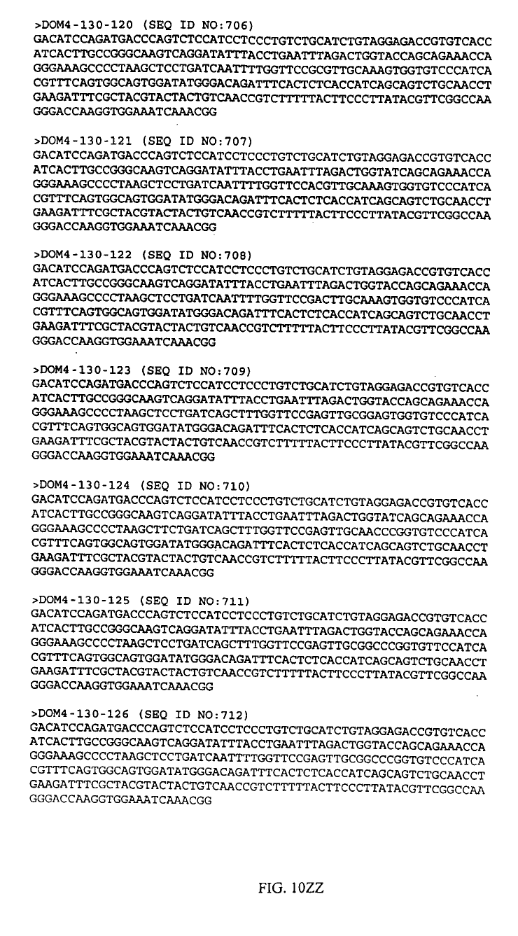

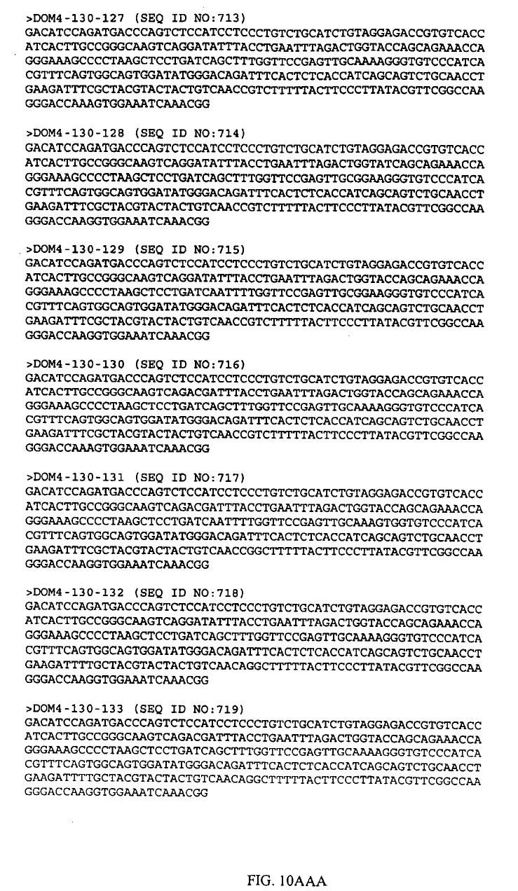

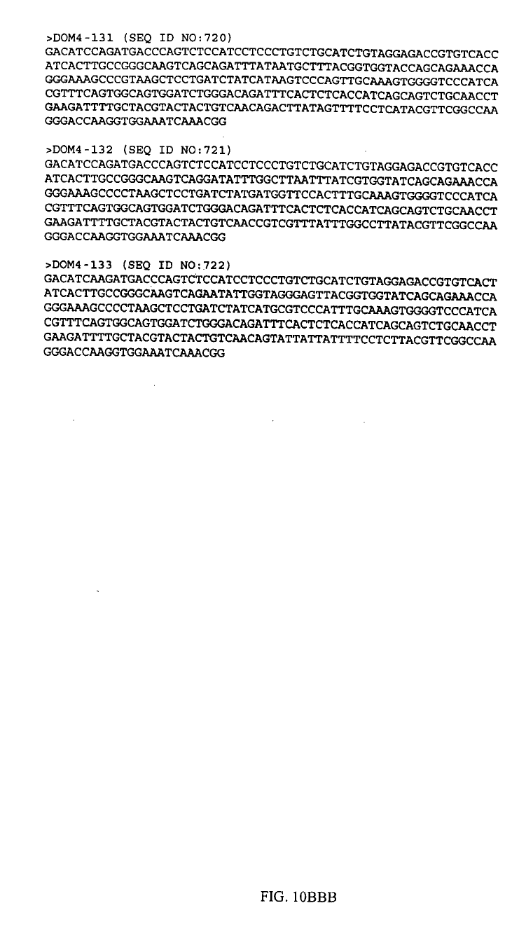

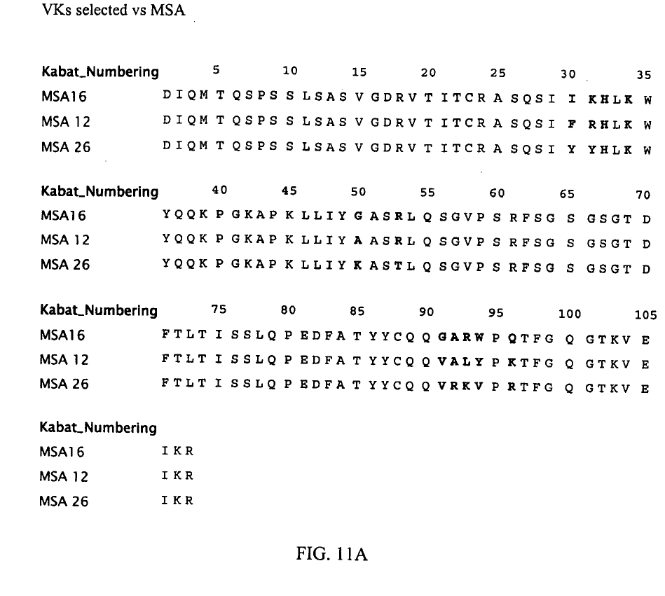

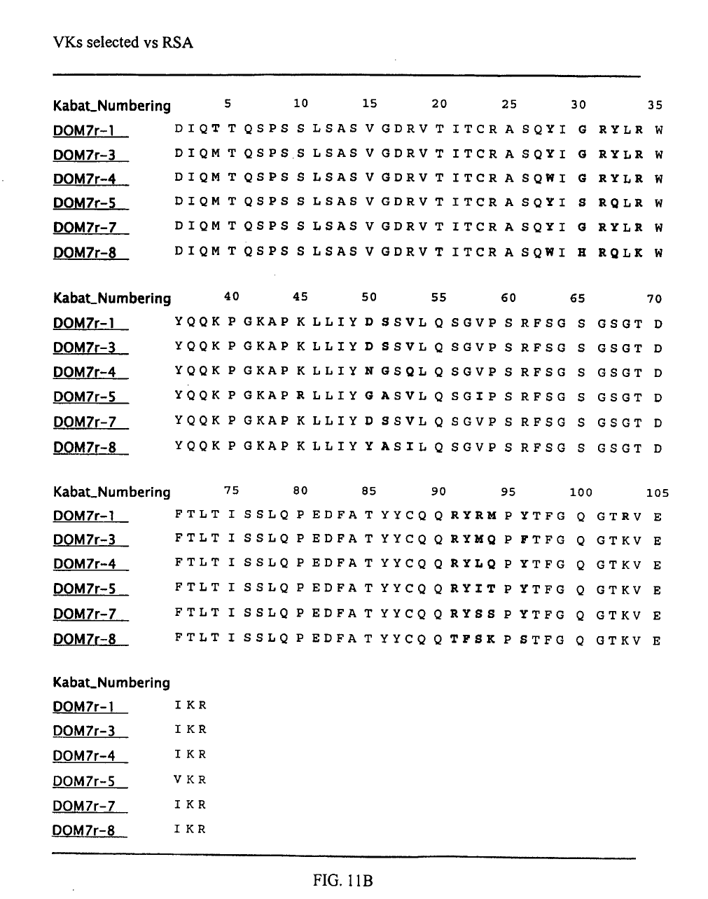

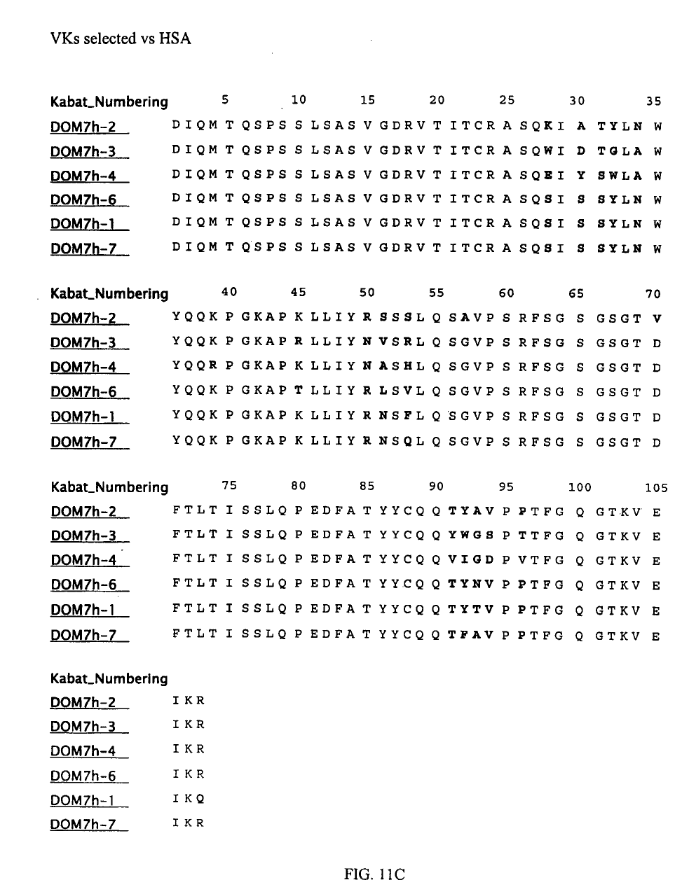

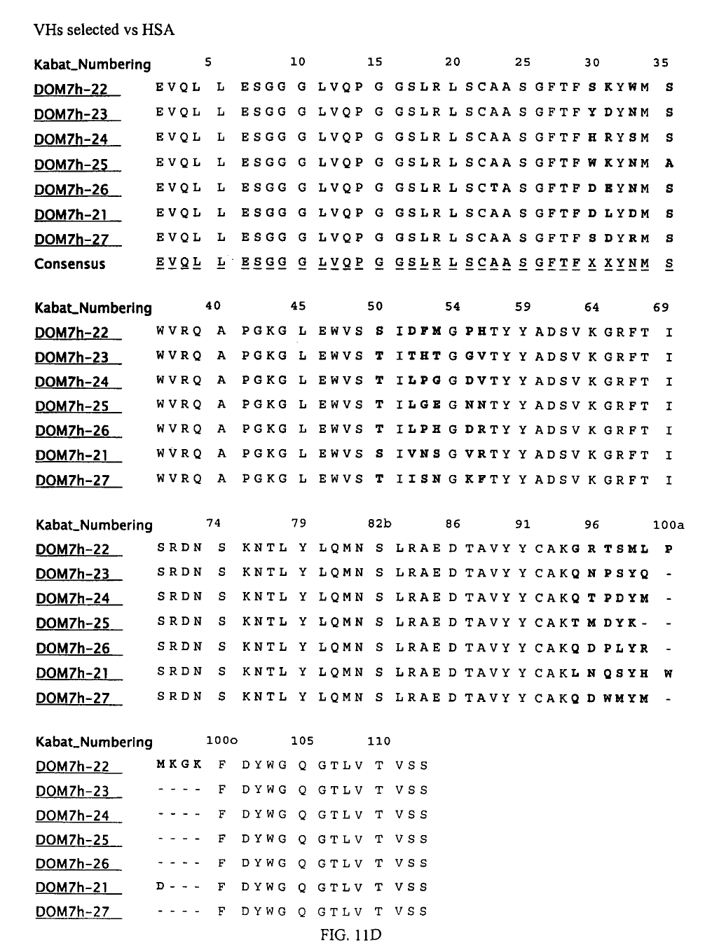

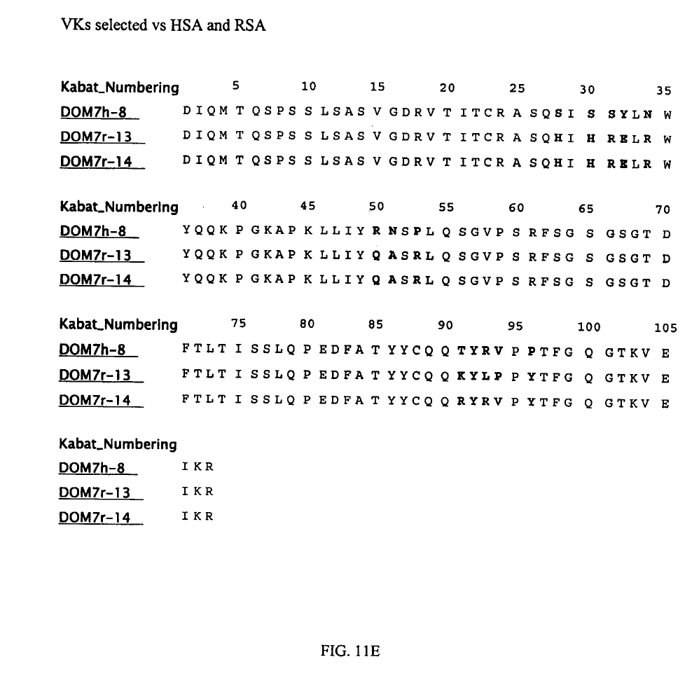

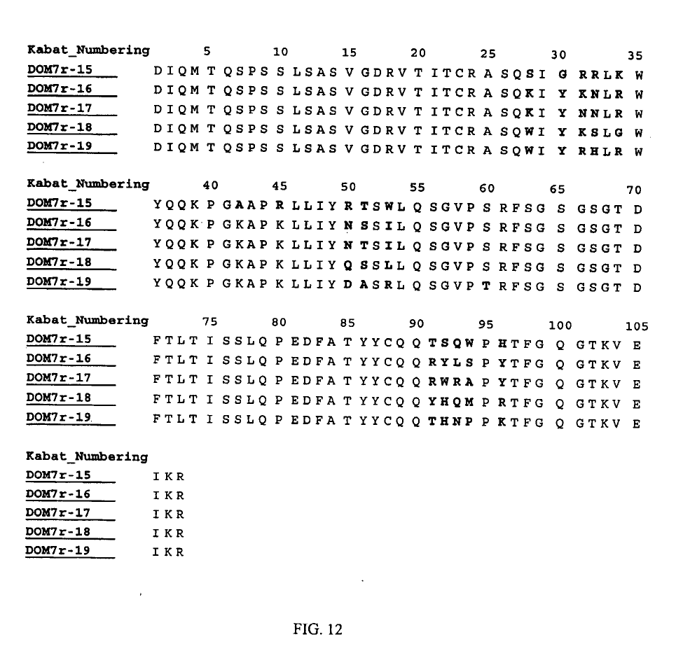

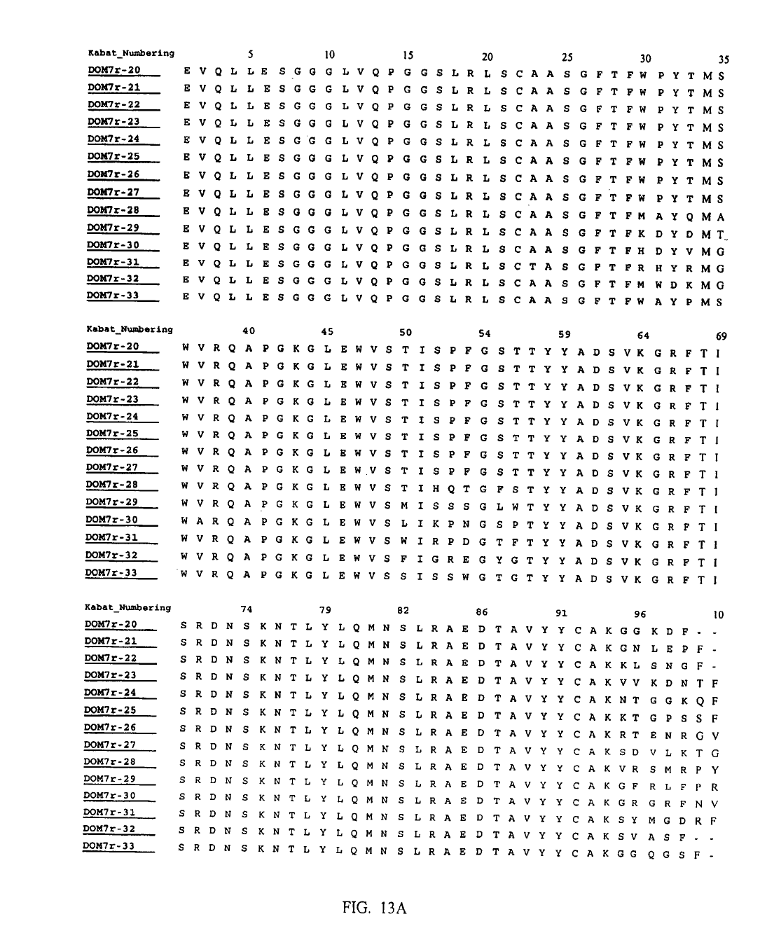

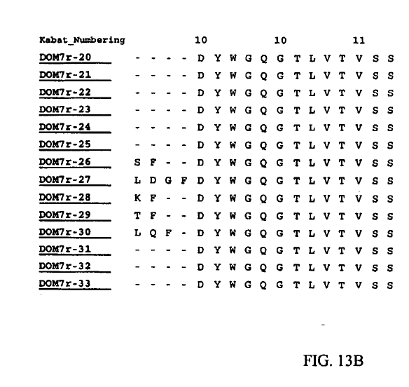

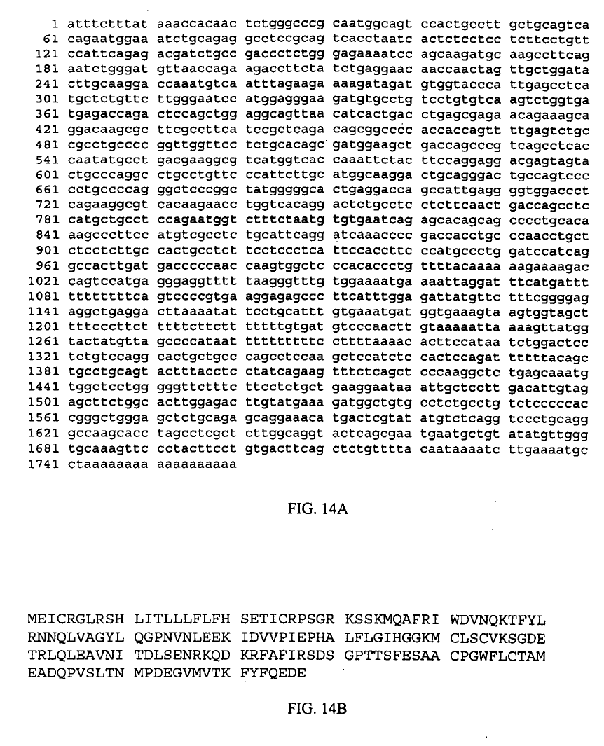

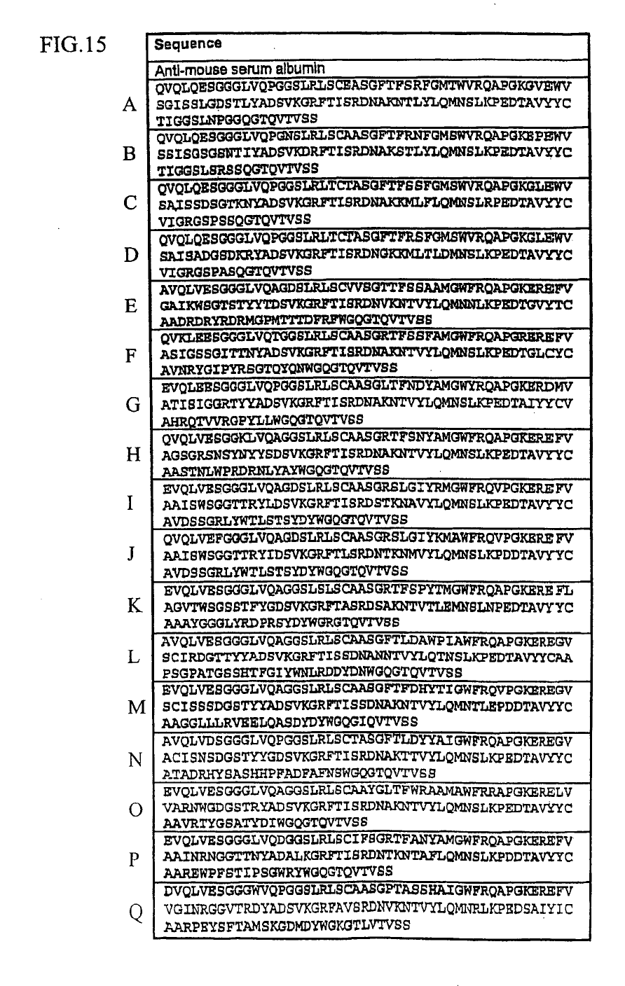

HEL-4 serum levels were detected in the 3 mg/kg and the 30 mg/kg dose groups (FIG. 7). The serum levels showed a similar pattern as the BAL levels. There appears to be a maximum level 2 hours after administration, followed by a slow decline. At 2 hours after administration, maximum levels of 3.5 μg/ml were detected in the serum with the 30 mg/kg dosing. This means that about 1% (5 μg of 600 μg administered) of the administered material was detected in the serum.

### Example 4

### Local Administration of an Antagonist of IL-1R1 to Pulmonary Tissue

In this study the antagonist of IL-1R1, KINARET® (anakinra; Amgen) a recombinant, nonglycosylated form of the human interleukin-1 receptor antagonist (IL-1Ra) that differs from native human IL-1Ra in that it has the addition of a single methionine residue at its amino terminus, was administered by intra-nasal administration, and pharmacokinetics were evaluated.

### Methods

KINARET® (anakinra; Amgen) was diluted in 20 mM sodium citrate pH6.0, 100 mM NaCl. All animals were treated on the same day within 1 to 2 hours of warming the solution.

Female mice (C57BL/6) full barrier bred and certified free of specific micro organisms on receipt (16-20 g) (Charles River) were housed in groups of up to 5 in individually ventilated, solid bottomed cages (IVC) with aspen chip bedding. Environments (airflow, temperature and humidity) within the cages were controlled by the IVC system (Techniplast).

There were 5 treatment groups, and each group contained 3 animals. The treatment groups are summarized in Table 3. All treatments were administered intranasally, and the dose volume was 50 microliters (25 microliteres per nare). Mice were sacrificed 1, 2, 5, 8, or 24 hours after administration.

TABLE 3

| Group | | Concentration | Sacrifice time after |
|---|---|---|---|
| No. | Dose | mg/ml | administration |
| 1 | 1 mg/kg | 0.4 mg/ml | 1 |
| 2 | 1 mg/kg | 0.4 mg/ml | 2 |
| 3 | 1 mg/kg | 0.4 mg/ml | 5 |
| 4 | 1 mg/kg | 0.4 mg/ml | 8 |
| 5 | 1 mg/kg | 0.4 mg/ml | 24 |

### ELISA to Detect IL1ra.

A 96 well Maxisorp (Nunc) assay plate was coated overnight at 4° C. with 50 μl per well with mouse anti-human IL1R1 antibody (R&D systems) at 4 μg/ml in carbonate coating buffer pH 9.4. Wells were washed 3 times with 0.05% tween/PBS and 3 times with PBS. 200 μl per well of 1% BSA in PBS was added to block the plate for 1 hour. Wells were washed and then 100 μl of IL1R1 at 500 ng/ml (R&D systems) was added in 0.1% BSA/0.05% tween/PBS for 1 hour. Wells were washed and then 100 μl of IL1ra standard or sample was added in 0.1% BSA/0.05% tween/PBS. IL1ra standard and samples were incubated with the receptor for 30 minutes. IL-11 was then added (R&D Systems) at a final concentration of 4 ng/mL and plates were incubated for another hour. Wells were washed and bound IL-1 was detected with biotinylated anti IL-1β antibody (R&D systems) at 0.5 μg/ml in 0.1% BSA/0.05% tween/PBS for 1 hour. Wells were washed and then 100 μl of anti-biotin-HRP antibody was added (Stratech)(1/5000 in 0.1% BSA/0.05% tween/PBS) for 1 hour. Plates were developed with 100 μl of SureBlue 1-Component TMB MicroWell Peroxidase (KPL, Gaithersburg, USA) solution was added to each well, and the plate was left at room temperature until a suitable signal has developed (˜15 minutes). The reaction was stopped by the addition of HCl and the absorbance was read at 450 nm.

### Results

The level in the BAL (FIG. 8) was maximum at 1 hour after administration and was ˜11 μg/ml (˜2.75 μg in 0.25 ml of BAL fluid). This means that at least 14% (2.75 μg of 20 μg total administered) of the adminstered material is delivered in the lung. More material will be present in the surrounding tissues but this cannot be recovered. The levels in the BAL are high for a prolonged period of time and show a gradual decline over 24 hrs. (>10-fold decline after 24 hrs).

The levels in the lung (FIG. 8) is maximum at 1 hr and was ˜3.3 μg/ml. This means that at least 16% (3.3 μg of 20 μg total administered) of the administered material is present in the lung. The levels in the lung are high for a prolonged period of time and show a gradual decline over 24 hrs. (>10-fold decline after 24 hrs).

The level in the serum (FIG. 8) at 1 hr was ˜260 ng/ml. At 5 hrs the levels in the serum was maximum (350 ng/ml). This means that the percentage of the total delivered dose present in the serum at 5 hrs is ˜2.6% (Total dose administered was 20 μg; 1.5 ml of blood volume). The levels in the serum show a slow decline and after 24 hrs there is only a 5-fold decline in the levels.

### Example 5

### Local Administration of Antagonists of IL-1R1 to Pulmonary Tissue in a Subchronic Model of COPD in C57BL/6 Mice

In view of the demonstrated ability to locally administer an antagonist to the lung by intranasal administration, a pilot study to assess this delivery route in a disease model was conducted. In this study, Il1ra was administered by the intra-nasal route 1 hour prior to each air or TS exposure. The effects on tobacco smoke (TS) induced changes in pulmonary inflammatory indices induced by 11 consecutive daily TS exposures was examined 24 h following the final exposure. The anti-TNF compound ENBREL® (etanercept; Immunex Corporation) was used as a positive control.

Test Substance 1: ENBREL® (etanercept; Immunex Corporation)
Test Substance 2: KINARET® (anakinra; Amgen)
The vehicle was sterile sodium citrate pH6.0, 100 mM NaCl.

### Methods

Female mice (C57BL/6) full barrier bred and certified free of specific micro organisms on receipt (16-20 g) (Charles River) were housed in groups of up to 5 in individually ventilated, solid bottomed cages (IVC) with aspen chip bedding. Environments (airflow, temperature and humidity) within the cages were controlled by the IVC system (Techniplast).

There were 4 treatment groups, and each group contained 10 animals. The treatment groups are summarized in Table 4. All treatments were administered intranasally, and the dose volume was 50 microliters (25 microliteres per nare). Mice were sacrificed 1, 2, 5, 8, or 24 hours after administration. Treatments were administered every 1 hour prior to each TS or air exposure.

TABLE 4

| Group | TS/Air | Compound | Dose |
|---|---|---|---|
| No. | Exposure | No. | mg/kg |
| 1 | Air | Vehicle | 0 |
| 2 | TS | Vehicle | 0 |
| 3 | TS | 1 | 1.0 |
| 4 | TS | 2 | 1.0 |

### TS Exposure

Mice (maximum 5 per exposure chamber) were exposed to TS generated from cigarettes (Type 1R1, supplied by University of Kentucky). Initial exposure was to 4 cigarettes on day 1, increasing to a maximum of 6 cigarettes per day by day 6/7. Exposure thereafter to Day 11 was to 6 cigarettes per day. The rate of increase was regulated with regard to the daily observed tolerance of the mice. The control group of mice was exposed to air for an equivalent length of time on each exposure day (Air exposure).

### Health Monitoring:

Animals were weighed prior to the start of the study, on day 6 of the exposure protocol, and at the time of termination. All animals were monitored during and after each test substance administration and TS exposure.

### Terminal Procedures:

Animals were sacrificed by anaesthetic overdose (pentobarbitone Na, 100 mg/kg i.p.) as follows: All groups were sacrificed 24 hours after the 11^{th} and final TS exposure. Mice from all treatment groups were treated as follows: Blood samples were taken from the sub-clavian artery, placed in a microcentrifuge tube and allowed to clot overnight at 4° C. The clot was removed and the remaining fluid was centrifuged at 2900 rpm in a microcentrifuge for 6 minutes. The resulting supernatant serum was decanted and stored at −40° C. for possible PK analysis. A bronchoalveolar lavage (BAL) was performed using 0.4 ml of phosphate buffered saline (PBS). Cells recovered from the BAL were quantified by total and differential cell counts. Lungs were removed, snap frozen in liquid nitrogen and stored at −80° C. for possible PK analysis

### Data Analysis

A test for normality was carried out on the data. If the test was positive, then a preliminary analysis was carried out using a one way analysis of variance test (one way ANOVA) followed by a Bonferroni's multiple comparison post test to compare control and treatment groups. If the data was not normally distributed, then a Kruskal-Wallis test followed by Dunn's multiple comparisons test was employed. Data were considered significant when p<0.05.

### Results

This pilot study was conducted to evaluate local delivery of antagonists of IL-1R1 to pulmonary tissue in a model of respiratory disease. The results showed that the level of total cells in group treated with KINARET® (anakinra; Amgen) were 29% lower (and 11% lower in the group treated with ENBREL® (etanercept; Immunex Corporation)) than in the control group that was exposed to TS by administered vehicle. Although the changes observed in this pilot study did not achieve statistical significance, the results indicate that polypeptide antagonists can be locally administered to pulmonary tissue.

While this invention has been particularly shown and described with references to preferred embodiments thereof, it will be understood by those skilled in the art that various changes in form and details may be made therein without departing from the scope of the invention encompassed by the appended claims.

What is claimed is:

1. Use of an antagonist of Interleukin-1 Receptor Type 1 (IL-1R1) for the manufacture of a medicament for treating a respiratory disease, wherein said antagonist of IL-1R1 comprises a polypeptide domain that has binding specificity for Interleukin-1 Receptor Type 1 (IL-1R1) and inhibits binding of Interleukin-1 (IL-1) to IL-1R1, and wherein said polypeptide domain that has binding specificity for IL-1R1 is provided by an antibody or antigen-binding fragment thereof, Interleukin-1 receptor antagonist (IL-1ra) or a functional variant of IL-1ra.

2. The use of claim 1, wherein said polypeptide domain that has binding specificity for IL-1R1 inhibits binding of IL-1 to IL-1R1 with an IC50 that is ≦1 μM.

3. The use of claim 1, wherein said polypeptide domain that has binding specificity for IL-1R1 inhibits IL-1-induced release of Interleukin-8 by MRC-5 cells (ATCC Accession No. CCL-171) in an in vitro assay with a ND50 that is ≦1 μM.

4. The use of claim 4, wherein said polypeptide domain that has binding specificity for IL-1R1 inhibits IL-1-induced release of Interleukin-8 by MRC-5 cells (ATCC Accession No. CCL-171) in an in vitro assay with a ND50 that is ≦1 nM.

5. The use of claim 1, wherein said polypeptide domain that has binding specificity for IL-1R1 inhibits IL-1-induced release of Interleukin-6 in a whole blood assay with a ND50 that is ≦1 μM.

6. The use of any one of claims 1-5, wherein said polypeptide domain that has binding specificity for IL-1R1 is an antigen-binding fragment of an antibody, and said antigen-binding fragment is an immunoglobulin single variable domain.

7. The use of claim 6, wherein one or more of the framework regions (FR) in said immunoglobulin single variable domain comprise (a) the amino acid sequence of a human framework region, (b) at least 8 contiguous amino acids of the amino acid sequence of a human framework region, or (c) an amino acid sequence encoded by a human germline antibody gene segment, wherein said framework regions are as defined by Kabat.

8. The use of claim 6, wherein the amino acid sequences of one or more framework regions in said immunoglobulin single variable domain are the same as the amino acid sequence of a corresponding framework region encoded by a human germline antibody gene segment, or the amino acid sequences of one or more of said framework regions collectively comprise up to 5 amino acid differences relative to the corresponding framework regions encoded by a human germline antibody gene segment.

9. The use of claim 6, wherein the amino acid sequences of FR1, FR2, FR3 and FR4 in said immunoglobulin single variable domain are the same as the amino acid sequences of corresponding framework regions encoded by a human germline antibody gene segment, or the amino acid sequences of FR1, FR2, FR3 and FR4 collectively contain up to 10 amino acid differences relative to the corresponding framework regions encoded by a human germline antibody gene segment.

10. The use of claim 6, wherein the immunoglobulin single variable domain comprises FR1, FR2 and FR3 regions, and the amino acid sequence of said FR1, FR2 and FR3 are the same as the amino acid sequences of corresponding framework regions encoded by a human germline antibody gene segment.

11. The use of any one of claims 7-10, wherein said human germline antibody gene segment comprises is DPK9 and JK1.

12. The use of any one of claims 6-11, wherein said immunoglobulin single variable domain competes for binding to IL-1R1 with an immunoglobulin single variable domain selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO: 103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173) DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO: 182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

13. The use of claim 12, wherein said immunoglobulin single variable domain comprises an amino acid sequence that has at least about 90% amino acid sequence identity with an amino acid sequence selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:13), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-1129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

14. The use of any one of claims 1-14, wherein said polypeptide domain that has binding specificity for IL-1R1 binds human IL-1R1 with an affinity (KD) of about 300 nM to about 5 pM, as determined by surface plasmon resonance.

15. The use of any one of claims 1-14, wherein said antagonist of IL-1R1 further comprises a half-life extending moiety.

16. The use of claim 15, wherein said half-life extending moiety is a polyalkylene glycol moiety, serum albumin or a fragment thereof, transferrin receptor or a transferrin-binding portion thereof, or an antibody or antibody fragment comprising a binding site for a polypeptide that enhances half-life in vivo.

17. The use of claim 16, wherein said half-life extending moiety is a polyethylene glycol moiety.

18. The use of claim 16, wherein said half-life extending moiety is an antibody or antibody fragment comprising a binding site for serum albumin or neonatal Fe receptor.

19. The use of claim 18, wherein said antibody or antibody fragment is an antibody fragment, and said antibody fragment is an immunoglobulin single variable domain.

20. The use of claim 19, wherein said immunoglobulin single variable domain competes with an immunoglobulin single variable domain selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7h-8 (SEQ ID NO:746), DOM7r-13 (SEQ ID NO:747), DOM7r-14 (SEQ ID NO:748), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), DOM7r-33 (SEQ ID NO:767), Sequence A (SEQ ID NO:768), Sequence B (SEQ ID NO:769), Sequence C (SEQ ID NO:770), Sequence D (SEQ ID NO:771), Sequence E (SEQ ID NO:772), Sequence F (SEQ ID NO:773), Sequence G (SEQ ID NO:774), Sequence H (SEQ ID NO:775), Sequence I (SEQ ID NO:776), Sequence J (SEQ ID NO:777), Sequence K (SEQ ID NO:778), Sequence L (SEQ ID NO:779), Sequence M (SEQ ID NO:780), Sequence N (SEQ ID NO:781), Sequence O (SEQ ID NO:782), Sequence P (SEQ ID NO:783), and Sequence Q (SEQ ID NO:784), for binding to human serum albumin.

21. The use of claim 20, wherein said immunoglobulin single variable domain binds human serum albumin comprises an amino acid sequence selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7h-8 (SEQ ID NO:746), DOM7r-13 (SEQ ID NO:747), DOM7r-14 (SEQ ID NO:748), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), DOM7r-33 (SEQ ID NO:767), Sequence A (SEQ ID NO:768), Sequence B (SEQ ID NO:769), Sequence C (SEQ ID NO:770), Sequence D (SEQ ID NO:771), Sequence E (SEQ ID NO:772), Sequence F (SEQ ID NO:773), Sequence G (SEQ ID NO:774), Sequence H (SEQ ID NO:775), Sequence I (SEQ ID NO:776), Sequence J (SEQ ID NO:777), Sequence K (SEQ ID NO:778), Sequence L (SEQ ID NO:779), Sequence M (SEQ ID NO:780), Sequence N (SEQ ID NO:781), Sequence O (SEQ ID NO:782), Sequence P (SEQ ID NO:783), and Sequence Q (SEQ ID NO:784).

22. The use of any one of claims 1-21, wherein said antagonist of IL-1R1 further comprises a polypeptide binding domain that has binding specificity for Tumor Necrosis Factor Receptor 1 (TNFR1, p55) and inhibits binding of Tumor Necrosis Factor Alpha (TNFα) to TNFR1.

23. The use of any one of claims 1-22, wherein said antagonist of IL-1R1 binds human IL-1R1 with an affinity (KD) of about 300 nM to about 5 pM, as determined by surface plasmon resonance.

24. The use of any one of claims 1-23, wherein said respiratory disease is selected from the group consisting of lung inflammation, chronic obstructive pulmonary disease, asthma, pneumonia, hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease, influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X, pulmonary hypertension, pulmonary nocardiosis, pulmonary tuberculosis, pulmonary veno-occlusive disease, rheumatoid lung disease, sarcoidosis, and Wegener's granulomatosis.

25. The use of any one of claims 1-24, wherein the medicament is for administration together with an antagonist of Tumor Necrosis Factor Receptor 1 (TNFR1, p55), or further comprises an antagonist of TNFR1.

26. The use of any one of claims 1-24, wherein said medicament is for treating a respiratory disease by systemic administration of the medicament.

27. The use of claim 26, wherein said medicament is for treating a respiratory disease by systemic administration of the medicament, wherein systemic administration is intraperoneal or subcutaneous administration.

28. The use of any one of claims 1-24, wherein said medicament is for treating a respiratory disease by local administration of said medicament to pulmonary tissue.

29. The use of claim 28, wherein s said medicament is for treating a respiratory disease by local administration of said medicament to pulmonary tissue by inhalation or intranasal administration.

30. The use of any one of claims 1-29, wherein the level of inflammatory cells in the lung is assessed by total cell counts in bronchoalveolar lavage, sputum or bronchial biopsy is reduced relative to pretreatment levels.

31. The use of claim 30, wherein the level of inflammatory cells in the lung is assessed by macrophage, polymorphonuclear, lymphocyte and/or eosinophil cell counts in bronchoalveolar lavage, sputum or bronchial biopsy.

32. Use of an antagonist of Interleukin-1 Receptor Type 1 (IL-1R1) for the manufacture of a medicament for treating a respiratory disease, wherein said antagonist of IL-1R1 is a fusion protein or a conjugate comprising an antagonist of IL-1R1 moiety and a half-life extending moiety, wherein said antagonist of IL-1R1 moiety binds human IL-1R1 and inhibits binding of Interleukin-1 to human IL-1R1, and said half-life extending moiety is a polypeptide binding moiety that contains a binding site with binding specificity for a polypeptide that enhances serum half-life in vivo.

33. The use of claim 32, wherein said antagonist of IL-1R1 moiety is human Interleukin 1 receptor antagonist (IL-1ra) or a functional variant of human IL-1ra.

34. The use of claim 33, wherein said antagonist of IL-1R1 moiety is an immunoglobulin single variable domain that competes for binding to IL-1R1 with an immunoglobulin single variable domain selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO: 11), DOM4-5 (SEQ ID NO: 12), DOM4-6 (SEQ ID NO: 13), DOM4-7 (SEQ ID NO:14), DOM4-8 (SEQ ID NO:15), DOM4-9 (SEQ ID NO:16), DOM4-10 (SEQ ID NO: 17), DOM4-11 (SEQ ID NO: 18), DOM4-12 (SEQ ID NO: 19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

35. The use of claim 33, wherein said antagonist of IL-1R1 moiety is an immunoglobulin single variable domain that comprises an amino acid sequence that has at least about 90% amino acid sequence identity with an amino acid sequence selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-1129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

36. The use of any one of claims 32-35, said half-life extending moiety is serum albumin or a fragment thereof, transferrin receptor or a transferrin-binding portion thereof, or an antibody or antibody fragment comprising a binding site for a polypeptide that enhances half-life in vivo.

37. The use of claim 36, wherein said half-life extending moiety is an antibody or antibody fragment comprising a binding site for serum albumin or neonatal Fc receptor.

38. The use of claim 37, wherein said antibody or antibody fragment is an antibody fragment, and said antibody fragment is an immunoglobulin single variable domain.

39. The use of claim 38, wherein said immunoglobulin single variable domain competes with an immunoglobulin single variable domain selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7h-8 (SEQ ID NO:746), DOM7r-13 (SEQ ID NO:747), DOM7r-14 (SEQ ID NO:748), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), DOM7r-33 (SEQ ID NO:767), Sequence A (SEQ ID NO:768), Sequence B (SEQ ID NO:769), Sequence C (SEQ ID NO:770), Sequence D (SEQ ID NO:771), Sequence E (SEQ ID NO:772), Sequence F (SEQ ID NO:773), Sequence G (SEQ ID NO:774), Sequence H (SEQ ID NO:775), Sequence I (SEQ ID NO:776), Sequence J (SEQ ID NO:777), Sequence K (SEQ ID NO:778), Sequence L (SEQ ID NO:779), Sequence M (SEQ ID NO:780), Sequence N (SEQ ID NO:781), Sequence O (SEQ ID NO:782), Sequence P (SEQ ID NO:783), and Sequence Q (SEQ ID NO:784) for binding to human serum albumin.

40. The use of claim 38, wherein said immunoglobulin single variable domain binds human serum albumin comprises an amino acid sequence selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7h-8 (SEQ ID NO:746), DOM7r-13 (SEQ ID NO:747), DOM7r-14 (SEQ ID NO:748), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), DOM7r-33 (SEQ ID NO:767), Sequence A (SEQ ID NO:768), Sequence B (SEQ ID NO:769), Sequence C (SEQ ID NO:770), Sequence D (SEQ ID NO:771), Sequence E (SEQ ID NO:772), Sequence F (SEQ ID NO:773), Sequence G (SEQ ID NO:774), Sequence H (SEQ ID NO:775), Sequence I (SEQ ID NO:776), Sequence J (SEQ ID NO:777), Sequence K (SEQ ID NO:778), Sequence L (SEQ ID NO:779), Sequence M (SEQ ID NO:780), Sequence N (SEQ ID NO:781), Sequence O (SEQ ID NO:782), Sequence P (SEQ ID NO:783), and Sequence Q (SEQ ID NO:784).

41. The use of any one of claims 32-40, wherein said respiratory disease is selected from the group consisting of lung inflammation, chronic obstructive pulmonary disease, asthma, pneumonia, hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease, influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X, pulmonary hypertension, pulmonary nocardiosis, pulmonary tuberculosis, pulmonary veno-occlusive disease, rheumatoid lung disease, sarcoidosis, and Wegener's granulomatosis.

42. Use of an antagonist of Interleukin-1 Receptor Type 1 (IL-1R1) for the manufacture of a medicament for treating a respiratory disease, wherein said antagonist of IL-1R1 comprises an immunoglobulin single variable domain that has binding specificity for human IL-1R1 and inhibits binding of Interleukin-1 (IL-1) to human IL-1R1, and a polyethylene glycol moiety.

43. The use of claim 42, wherein said immunoglobulin single variable domain competes for binding to human IL-1R1 with an immunoglobulin single variable domain selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

44. The use of claim 42, wherein said immunoglobulin single variable domain binds human serum albumin comprises an amino acid sequence selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

45. The use of any one of claims 42-44, wherein said respiratory disease is selected from the group consisting of lung inflammation, chronic obstructive pulmonary disease, asthma, pneumonia, hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease, influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X, pulmonary hypertension, pulmonary nocardiosis, pulmonary tuberculosis, pulmonary veno-occlusive disease, rheumatoid lung disease, sarcoidosis, and Wegener's granulomatosis.

46. The use of any one of claims 1-45 wherein said IL-1 is selected from the group consisting of IL-1α and IL-1β.

47. A pharmaceutical composition comprising an antagonist of IL-1R1 and a physiologically acceptable carrier, wherein said antagonist of IL-1R1 is as described in any one of the previous claims.

48. A drug delivery device comprising the pharmaceutical composition of claims 47.

49. The drug deliver device of claim 48 wherein said drug delivery device is selected from the group consisting of a parenteral delivery device, intravenous delivery device, intramuscular delivery device, intraperitoneal delivery device, transdermal delivery device, pulmonary delivery device, intraarterial delivery device, intrathecal delivery device, intraarticular delivery device, subcutaneous delivery device, intranasal delivery device, vaginal delivery device, and rectal delivery device.

50. The drug delivery device of claim 49 wherein said device is selected from the group consisting of a syringe, a transdermal delivery device, a capsule, a tablet, a nebulizer, an inhaler, an atomizer, an aerosolizer, a mister, a dry powder inhaler, a metered dose inhaler, a metered dose sprayer, a metered dose mister, a metered dose atomizer, a catheter.

51. A method for treating a respiratory disease, comprising administering to a subject in need thereof a therapeutically effective amount of an antagonist of Interleukin-1 Receptor Type 1 (IL-1R1), wherein said antagonist of IL-1R1 comprises a polypeptide domain that has binding specificity for Interleukin-1 Receptor Type 1 (IL-1R1) and inhibits binding of Interleukin-1 (IL-1) to IL-1R1, and wherein said polypeptide domain that has binding specificity for IL-1R1 is selected from the group consisting of an antibody or antigen-binding fragment thereof, Interleukin-1 receptor antagonist (IL-1ra) or a functional variant of IL-1ra.

52. The method of claim 51, wherein said polypeptide domain that has binding specificity for IL-1R1 inhibits binding of said ligand to IL-1R1 with an IC50 that is ≦1 μM.

53. The method of claim 51, wherein said polypeptide domain that has binding specificity for IL-1R1 inhibits IL-1-induced release of Interleukin-8 by MRC-5 cells (ATCC Accession No. CCL-171) in an in vitro assay with a ND50 that is ≦1 μM.

54. The method of claim 53, wherein said polypeptide domain that has binding specificity for IL-1R1 inhibits IL-1-induced release of Interleukin-8 by MRC-5 cells (ATCC Accession No. CCL-171) in an in vitro assay with a ND50 that is ≦1 nM.

55. The method of claim 51, wherein said polypeptide domain that has binding specificity for IL-1R1 inhibits IL-1-induced release of Interleukin-6 in a whole blood assay with a ND50 that is ≦1 μM.

56. The method of claim 51, wherein said polypeptide domain that has binding specificity for IL-1R1 is an antigen-binding fragment of an antibody, and said antigen-binding fragment is an immunoglobulin single variable domain.

57. The method of claim 56, wherein one or more of the framework regions (FR) in said immunoglobulin single variable domain comprise (a) the amino acid sequence of a human framework region, (b) at least 8 contiguous amino acids of the amino acid sequence of a human framework region, or (c) an amino acid sequence encoded by a human germline antibody gene segment, wherein said framework regions are as defined by Kabat.

58. The method of claim 56, wherein the amino acid sequences of one or more framework regions in said immunoglobulin single variable domain are the same as the amino acid sequence of a corresponding framework region encoded by a human germline antibody gene segment, or the amino acid sequences of one or more of said framework regions collectively comprise up to 5 amino acid differences relative to the corresponding framework regions encoded by a human germline antibody gene segment.

59. The method of claim 56, wherein the amino acid sequences of FR1, FR2, FR3 and FR4 in said immunoglobulin single variable domain are the same as the amino acid sequences of corresponding framework regions encoded by a human germline antibody gene segment, or the amino acid sequences of FR1, FR2, FR3 and FR4 collectively contain up to 10 amino acid differences relative to the corresponding framework regions encoded by a human germline antibody gene segment.

60. The method of claim 56, wherein the immunoglobulin single variable domain comprises FR1, FR2 and FR3 regions, and the amino acid sequence of said FR1, FR2 and FR3 are the same as the amino acid sequences of corresponding framework regions encoded by a human germline antibody gene segment.

61. The method of any one of claims 57-60, wherein said human germline antibody gene segment comprises is DPK9 and JK1.

62. The method of claim 56, wherein said immunoglobulin single variable domain competes for binding to IL-1R1 with an immunoglobulin single variable domain selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

63. The method of claim 56, wherein said immunoglobulin single variable domain comprises an amino acid sequence that has at least about 90% amino acid sequence identity with an amino acid sequence selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

64. The method of claim 51, wherein said polypeptide domain that has binding specificity for IL-1R1 binds human IL-1R1 with an affinity (KD) of about 300 nM to about 5 pM, as determined by surface plasmon resonance.

65. The method of claim 51, wherein said antagonist of IL-1R1 further comprises a half-life extending moiety.

66. The method of claim 65, wherein said half-life extending moiety is a polyalkylene glycol moiety, serum albumin or a fragment thereof, transferrin receptor or a transferrin-binding portion thereof, or an antibody or antibody fragment comprising a binding site for a polypeptide that enhances half-life in vivo.

67. The method of claim 66, wherein said half-life extending moiety is a polyethylene glycol moiety.

68. The method of claim 66, wherein said half-life extending moiety is an antibody or antibody fragment comprising a binding site for serum albumin or neonatal Fc receptor.

69. The method of claim 68, wherein said antibody or antibody fragment is an antibody fragment, and said antibody fragment is an immunoglobulin single variable domain.

70. The method of claim 69, wherein said immunoglobulin single variable domain competes with an immunoglobulin single variable domain selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7h-8 (SEQ ID NO:746), DOM7r-13 (SEQ ID NO:747), DOM7r-14 (SEQ ID NO:748), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), DOM7r-33 (SEQ ID NO:767), Sequence A (SEQ ID NO:768), Sequence B (SEQ ID NO:769), Sequence C (SEQ ID NO:770), Sequence D (SEQ ID NO:771), Sequence E (SEQ ID NO:772), Sequence F (SEQ ID NO:773), Sequence G (SEQ ID NO:774), Sequence H (SEQ ID NO:775), Sequence I (SEQ ID NO:776), Sequence J (SEQ ID NO:777), Sequence K (SEQ ID NO:778), Sequence L (SEQ ID NO:779), Sequence M (SEQ ID NO:780), Sequence N (SEQ ID NO:781), Sequence O (SEQ ID NO:782), Sequence P (SEQ ID NO:783), and Sequence Q (SEQ ID NO:784), for binding to human serum albumin.

71. The method of claim 69, wherein said immunoglobulin single variable domain binds human serum albumin comprises an amino acid sequence selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7h-8 (SEQ ID NO:746), DOM7r-13 (SEQ ID NO:747), DOM7r-14 (SEQ ID NO:748), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), DOM7r-33 (SEQ ID NO:767), Sequence A (SEQ ID NO:768), Sequence B (SEQ ID NO:769), Sequence C (SEQ ID NO:770), Sequence D (SEQ ID NO:771), Sequence E (SEQ ID NO:772), Sequence F (SEQ ID NO:773), Sequence G (SEQ ID NO:774), Sequence H (SEQ ID NO:775), Sequence I (SEQ ID NO:776), Sequence J (SEQ ID NO:777), Sequence K (SEQ ID NO:778), Sequence L (SEQ ID NO:779), Sequence M (SEQ ID NO:780), Sequence N (SEQ ID NO:781), Sequence O (SEQ ID NO:782), Sequence P (SEQ ID NO:783), and Sequence Q (SEQ ID NO:784).

72. The method of claim 51, wherein said antagonist of IL-1R1 further comprises a polypeptide binding domain that has binding specificity for Tumor Necrosis Factor Receptor 1 (TNFR1, p55) and inhibits binding of Tumor Necrosis Factor Alpha (TNFα) to TNFR1.

73. The method of claim 51, wherein said antagonist of IL-1R1 binds human IL-1R1 with an affinity (KD) of about 300 nM to about 5 pM, as determined by surface plasmon resonance.

74. The method of claim 51, wherein said respiratory disease is selected from the group consisting of lung inflammation, chronic obstructive pulmonary disease, asthma, pneumonia, hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease, influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X, pulmonary hypertension, pulmonary nocardiosis, pulmonary tuberculosis, pulmonary veno-occlusive disease, rheumatoid lung disease, sarcoidosis, and Wegener's granulomatosis.

75. The method of claim 51, further comprising administering to said subject a therapeutically effective amount of an antagonist of Tumor Necrosis Factor Receptor 1 (TNFR1, p55).

76. The method of claim 51, wherein said antagonist of IL-1R1 is administered systemically.

77. The method of claim 76, wherein said antagonist of IL-1R1 is administered intraperoneally or subcutaneously.

78. The method of claim 51, wherein said antagonist of IL-1R1 is locally administered to pulmonary tissue.

79. The method of claim 78, wherein said antagonist of IL-1R1 is administered by inhalation or intranasal administration.

80. The method of claim 51, wherein the level of inflammatory cells in the lung is assessed by total cell counts in bronchoalveolar lavage, sputum or bronchial biopsy is reduced relative to pretreatment levels.

81. The method of claim 80, wherein the level of inflammatory cells in the lung is assessed by macrophage, polymorphonuclear, lymphocyte and/or eosinophil cell counts in bronchoalveolar lavage, sputum or bronchial biopsy.

82. A method for treating a respiratory disease, comprising administering to a subject in need thereof a therapeutically effective amount of an antagonist of Interleukin-1 Receptor Type 1 (IL-1R1), wherein said antagonist of IL-1R1 is a fusion protein or a conjugate comprising an antagonist of IL-1R1 moiety and a half-life extending moiety, wherein said antagonist of IL-1R1 moiety binds human IL-1R1 and inhibits binding of Interleukin-1 (IL-1) to human IL-1R1, and said half-life extending moiety is a polypeptide binding moiety that contains a binding site with binding specificity for a polypeptide that enhances serum half-life in vivo.

83. The method of claim 82, wherein said antagonist of IL-1R1 moiety is human Interleukin 1 receptor antagonist (IL-1ra) or a functional variant of human IL-1 ra.

84. The method of claim 82, wherein said antagonist of IL-1R1 moiety is an immunoglobulin single variable domain that competes for binding to IL-1R1 with a dAb selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

85. The method of claim 82, wherein said antagonist of IL-1R1 moiety is an immunoglobulin single variable domain that comprises an amino acid sequence that has at least about 90% amino acid sequence identity with an amino acid sequence selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

86. The method of claim 82, said half-life extending moiety is serum albumin or a fragment thereof, transferrin receptor or a transferrin-binding portion thereof, or an antibody or antibody fragment comprising a binding site for a polypeptide that enhances half-life in vivo.

87. The method of claim 82, wherein said half-life extending moiety is an antibody or antibody fragment comprising a binding site for serum albumin or neonatal Fc receptor.

88. The method of claim 87, wherein said antibody or antibody fragment is an antibody fragment, and said antibody fragment is an immunoglobulin single variable domain.

89. The method of claim 88, wherein said immunoglobulin single variable domain competes with an immunoglobulin single variable domain selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7h-8 (SEQ ID NO:746), DOM7r-13 (SEQ ID NO:747), DOM7r-14 (SEQ ID NO:748), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), DOM7r-33 (SEQ ID NO:767), Sequence A (SEQ ID NO:768), Sequence B. (SEQ ID NO:769), Sequence C (SEQ ID NO:770), Sequence D (SEQ ID NO:771), Sequence E (SEQ ID NO:772), Sequence F (SEQ ID NO:773), Sequence G (SEQ ID NO:774), Sequence H (SEQ ID NO:775), Sequence I (SEQ ID NO:776), Sequence J (SEQ ID NO:777), Sequence K (SEQ ID NO:778), Sequence L (SEQ ID NO:779), Sequence M (SEQ ID NO:780), Sequence N (SEQ ID NO:781), Sequence O (SEQ ID NO:782), Sequence P (SEQ ID NO:783), and Sequence Q (SEQ ID NO:784) for binding to human serum albumin.

90. The method of claim 88, wherein said immunoglobulin single variable domain binds human serum albumin comprises an amino acid sequence selected from the group consisting of DOM7m-16 (SEQ ID NO:723), DOM7m-12 (SEQ ID NO:724), DOM7m-26 (SEQ ID NO:725), DOM7r-1 (SEQ ID NO:726), DOM7r-3 (SEQ ID NO:727), DOM7r-4 (SEQ ID NO:728), DOM7r-5 (SEQ ID NO:729), DOM7r-7 (SEQ ID NO:730), DOM7r-8 (SEQ ID NO:731), DOM7h-2 (SEQ ID NO:732), DOM7h-3 (SEQ ID NO:733), DOM7h-4 (SEQ ID NO:734), DOM7h-6 (SEQ ID NO:735), DOM7h-1 (SEQ ID NO:736), DOM7h-7 (SEQ ID NO:737), DOM7h-22 (SEQ ID NO:739), DOM7h-23 (SEQ ID NO:740), DOM7h-24 (SEQ ID NO:741), DOM7h-25 (SEQ ID NO:742), DOM7h-26 (SEQ ID NO:743), DOM7h-21 (SEQ ID NO:744), DOM7h-27 (SEQ ID NO:745), DOM7h-8 (SEQ ID NO:746), DOM7r-13 (SEQ ID NO:747), DOM7r-14 (SEQ ID NO:748), DOM7r-15 (SEQ ID NO:749), DOM7r-16 (SEQ ID NO:750), DOM7r-17 (SEQ ID NO:751), DOM7r-18 (SEQ ID NO:752), DOM7r-19 (SEQ ID NO:753), DOM7r-20 (SEQ ID NO:754), DOM7r-21 (SEQ ID NO:755), DOM7r-22 (SEQ ID NO:756), DOM7r-23 (SEQ ID NO:757), DOM7r-24 (SEQ ID NO:758), DOM7r-25 (SEQ ID NO:759), DOM7r-26 (SEQ ID NO:760), DOM7r-27 (SEQ ID NO:761), DOM7r-28 (SEQ ID NO:762), DOM7r-29 (SEQ ID NO:763), DOM7r-30 (SEQ ID NO:764), DOM7r-31 (SEQ ID NO:765), DOM7r-32 (SEQ ID NO:766), DOM7r-33 (SEQ ID NO:767), Sequence A (SEQ ID NO:768), Sequence B (SEQ ID NO:769), Sequence C (SEQ ID NO:770), Sequence D (SEQ ID NO:771), Sequence E (SEQ ID NO:772), Sequence F (SEQ ID NO:773), Sequence G (SEQ ID NO:774), Sequence H (SEQ ID NO:775), Sequence I (SEQ ID NO:776), Sequence J (SEQ ID NO:777), Sequence K (SEQ ID NO:778), Sequence L (SEQ ID NO:779), Sequence M (SEQ ID NO:780), Sequence N (SEQ ID NO:781), Sequence O (SEQ ID NO:782), Sequence P (SEQ ID NO:783), and Sequence Q (SEQ ID NO:784).

91. The method of claim 82, wherein said respiratory disease is selected from the group consisting of lung inflammation, chronic obstructive pulmonary disease, asthma, pneumonia, hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease, influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X, pulmonary hypertension, pulmonary nocardiosis, pulmonary tuberculosis, pulmonary veno-occlusive disease, rheumatoid lung disease, sarcoidosis, and Wegener's granulomatosis.

92. A method for treating a respiratory disease, comprising administering to a subject in need thereof a therapeutically effective amount of an antagonist of Interleukin-1 Receptor Type 1 (IL-1R1), wherein said antagonist of IL-1R1 comprises an immunoglobulin single variable domain that has binding specificity for human IL-1R1 and inhibits binding of Interleukin-1 (IL-1) to human IL-1R1, and a polyethylene glycol moiety.

93. The method of claim 92, wherein said immunoglobulin single variable domain competes for binding to human IL-1R1 with a dAb selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:1), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ TD NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO:131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

94. The method of claim 92, wherein said immunoglobulin single variable domain binds human serum albumin comprises an amino acid sequence selected from the group consisting of DOM4-122-23 (SEQ ID NO:1), DOM4-122-24 (SEQ ID NO:2), DOM4-130-30 (SEQ ID NO:3), DOM4-130-46 (SEQ ID NO:4), DOM4-130-51 (SEQ ID NO:5), DOM4-130-53 (SEQ ID NO:6), DOM4-130-54 (SEQ ID NO:7), DOM4-1 (SEQ ID NO:8), DOM4-2 (SEQ ID NO:9), DOM4-3 (SEQ ID NO:10), DOM4-4 (SEQ ID NO:11), DOM4-5 (SEQ ID NO:12), DOM4-6 (SEQ ID NO:13), DOM4-7 (SEQ ID NO:14), DOM4-8 (SEQ ID NO:15), DOM4-9 (SEQ ID NO:16), DOM4-10 (SEQ ID NO:17), DOM4-11 (SEQ ID NO:18), DOM4-12 (SEQ ID NO:19), DOM4-13 (SEQ ID NO:20), DOM4-14 (SEQ ID NO:21), DOM4-15 (SEQ ID NO:22), DOM4-20 (SEQ ID NO:23), DOM4-21 (SEQ ID NO:24), DOM4-22 (SEQ ID NO:25), DOM4-23 (SEQ ID NO:26), DOM4-25 (SEQ ID NO:27), DOM4-26 (SEQ ID NO:28), DOM4-27 (SEQ ID NO:29), DOM4-28 (SEQ ID NO:30), DOM4-29 (SEQ ID NO:31), DOM4-31 (SEQ ID NO:32), DOM4-32 (SEQ ID NO:33), DOM4-33 (SEQ ID NO:34), DOM4-34 (SEQ ID NO:35), DOM4-36 (SEQ ID NO:36), DOM4-37 (SEQ ID NO:37), DOM4-38 (SEQ ID NO:38), DOM4-39 (SEQ ID NO:39), DOM4-40 (SEQ ID NO:40), DOM4-41 (SEQ ID NO:41), DOM4-42 (SEQ ID NO:42), DOM4-44 (SEQ ID NO:43), DOM4-45 (SEQ ID NO:44), DOM4-46 (SEQ ID NO:45), DOM4-49 (SEQ ID NO:46), DOM4-50 (SEQ ID NO:47), DOM4-74 (SEQ ID NO:48), DOM4-75 (SEQ ID NO:49), DOM4-76 (SEQ ID NO:50), DOM4-78 (SEQ ID NO:51), DOM4-79 (SEQ ID NO:52), DOM4-80 (SEQ ID NO:53), DOM4-81 (SEQ ID NO:54), DOM4-82 (SEQ ID NO:55), DOM4-83 (SEQ ID NO:56), DOM4-84 (SEQ ID NO:57), DOM4-85 (SEQ ID NO:58), DOM4-86 (SEQ ID NO:59), DOM4-87 (SEQ ID NO:60), DOM4-88 (SEQ ID NO:61), DOM4-89 (SEQ ID NO:62), DOM4-90 (SEQ ID NO:63), DOM4-91 (SEQ ID NO:64), DOM4-92 (SEQ ID NO:65), DOM4-93 (SEQ ID NO:66), DOM4-94 (SEQ ID NO:67), DOM4-95 (SEQ ID NO:68), DOM4-96 (SEQ ID NO:69), DOM4-97 (SEQ ID NO:70), DOM4-98 (SEQ ID NO:71), DOM4-99 (SEQ ID NO:72), DOM4-100 (SEQ ID NO:73), DOM4-101 (SEQ ID NO:74), DOM4-102 (SEQ ID NO:75), DOM4-103 (SEQ ID NO:76), DOM4-104 (SEQ ID NO:77), DOM4-105 (SEQ ID NO:78), DOM4-106 (SEQ ID NO:79), DOM4-107 (SEQ ID NO:80), DOM4-108 (SEQ ID NO:81), DOM4-109 (SEQ ID NO:82), DOM4-110 (SEQ ID NO:83), DOM4-111 (SEQ ID NO:84), DOM4-112 (SEQ ID NO:85), DOM4-113 (SEQ ID NO:86), DOM4-114 (SEQ ID NO:87), DOM4-115 (SEQ ID NO:88), DOM4-116 (SEQ ID NO:89), DOM4-117 (SEQ ID NO:90), DOM4-118 (SEQ ID NO:91), DOM4-119 (SEQ ID NO:92), DOM4-120 (SEQ ID NO:93), DOM4-121 (SEQ ID NO:94), DOM4-122 (SEQ ID NO:95), DOM4-122-1 (SEQ ID NO:96), DOM4-122-2 (SEQ ID NO:97), DOM4-122-3 (SEQ ID NO:98), DOM4-122-4 (SEQ ID NO:99), DOM4-122-5 (SEQ ID NO:100), DOM4-122-6 (SEQ ID NO:101), DOM4-122-7 (SEQ ID NO:102), DOM4-122-8 (SEQ ID NO:103), DOM4-122-9 (SEQ ID NO:104), DOM4-122-10 (SEQ ID NO:105), DOM4-122-11 (SEQ ID NO:106), DOM4-122-12 (SEQ ID NO:107), DOM4-122-13 (SEQ ID NO:108), DOM4-122-14 (SEQ ID NO:109), DOM4-122-15 (SEQ ID NO:110), DOM4-122-16 (SEQ ID NO:111), DOM4-122-17 (SEQ ID NO:112), DOM4-122-18 (SEQ ID NO:113), DOM4-122-19 (SEQ ID NO:114), DOM4-122-20 (SEQ ID NO:115), DOM4-122-21 (SEQ ID NO:116), DOM4-122-22 (SEQ ID NO:117), DOM4-122-25 (SEQ ID NO:118), DOM4-122-26 (SEQ ID NO:119), DOM4-122-27 (SEQ ID NO:120), DOM4-122-28 (SEQ ID NO:121), DOM4-122-29 (SEQ ID NO:122), DOM4-122-30 (SEQ ID NO:123), DOM4-122-31 (SEQ ID NO:124), DOM4-122-32 (SEQ ID NO:125), DOM4-122-33 (SEQ ID NO:126), DOM4-122-34 (SEQ ID NO:127), DOM4-122-35 (SEQ ID NO:128), DOM4-122-36 (SEQ ID NO:129), DOM4-122-37 (SEQ ID NO:130), DOM4-122-38 (SEQ ID NO: 131), DOM4-122-39 (SEQ ID NO:132), DOM4-122-40 (SEQ ID NO:133), DOM4-122-41 (SEQ ID NO:134), DOM4-122-42 (SEQ ID NO:135), DOM4-122-43 (SEQ ID NO:136), DOM4-122-44 (SEQ ID NO:137), DOM4-122-45 (SEQ ID NO:138), DOM4-122-46 (SEQ ID NO:139), DOM4-122-47 (SEQ ID NO:140), DOM4-122-48 (SEQ ID NO:141), DOM4-122-49 (SEQ ID NO:142), DOM4-122-50 (SEQ ID NO:143), DOM4-122-51 (SEQ ID NO:144), DOM4-122-52 (SEQ ID NO:145), DOM4-122-54 (SEQ ID NO:146), DOM4-122-55 (SEQ ID NO:147), DOM4-122-56 (SEQ ID NO:148), DOM4-122-57 (SEQ ID NO:149), DOM4-122-58 (SEQ ID NO:150), DOM4-122-59 (SEQ ID NO:151), DOM4-122-60 (SEQ ID NO:152), DOM4-122-61 (SEQ ID NO:153), DOM4-122-62 (SEQ ID NO:154), DOM4-122-63 (SEQ ID NO:155), DOM4-122-64 (SEQ ID NO:156), DOM4-122-65 (SEQ ID NO:157), DOM4-122-66 (SEQ ID NO:158), DOM4-122-67 (SEQ ID NO:159), DOM4-122-68 (SEQ ID NO:160), DOM4-122-69 (SEQ ID NO:161), DOM4-122-70 (SEQ ID NO:162), DOM4-122-71 (SEQ ID NO:163), DOM4-122-72 (SEQ ID NO:164), DOM4-122-73 (SEQ ID NO:165), DOM4-123 (SEQ ID NO:166), DOM4-124 (SEQ ID NO:167) DOM4-125 (SEQ ID NO:168), DOM4-126 (SEQ ID NO:169), DOM4-127 (SEQ ID NO:170), DOM4-128 (SEQ ID NO:171), DOM4-129 (SEQ ID NO:172), DOM4-129-1 (SEQ ID NO:173), DOM4-129-2 (SEQ ID NO:174), DOM4-129-3 (SEQ ID NO:175), DOM4-129-4 (SEQ ID NO:176), DOM4-129-5 (SEQ ID NO:177), DOM4-129-6 (SEQ ID NO:178), DOM4-129-7 (SEQ ID NO:179), DOM4-129-8 (SEQ ID NO:180), DOM4-129-9 (SEQ ID NO:181), DOM4-129-10 (SEQ ID NO:182), DOM4-129-11 (SEQ ID NO:183), DOM4-129-12 (SEQ ID NO:184), DOM4-129-13 (SEQ ID NO:185), DOM4-129-14 (SEQ ID NO:186), DOM4-129-15 (SEQ ID NO:187), DOM4-129-16 (SEQ ID NO:188), DOM4-129-17 (SEQ ID NO:189), DOM4-129-18 (SEQ ID NO:190), DOM4-129-19 (SEQ ID NO:191), DOM4-129-20 (SEQ ID NO:192), DOM4-129-21 (SEQ ID NO:193), DOM4-129-22 (SEQ ID NO:194), DOM4-129-23 (SEQ ID NO:195), DOM4-129-24 (SEQ ID NO:196), DOM4-129-25 (SEQ ID NO:197), DOM4-129-26 (SEQ ID NO:198), DOM4-129-27 (SEQ ID NO:199), DOM4-129-28 (SEQ ID NO:200), DOM4-129-29 (SEQ ID NO:201), DOM4-129-31 (SEQ ID NO:202), DOM4-129-32 (SEQ ID NO:203), DOM4-129-33 (SEQ ID NO:204), DOM4-129-34 (SEQ ID NO:205), DOM4-129-35 (SEQ ID NO:206), DOM4-129-37 (SEQ ID NO:207), DOM4-129-38 (SEQ ID NO:208), DOM4-129-39 (SEQ ID NO:209), DOM4-129-40 (SEQ ID NO:210), DOM4-129-41 (SEQ ID NO:211), DOM4-129-42 (SEQ ID NO:212), DOM4-129-43 (SEQ ID NO:213), DOM4-129-44 (SEQ ID NO:214), DOM4-130 (SEQ ID NO:215), DOM4-130-1 (SEQ ID NO:216), DOM4-130-2 (SEQ ID NO:217), DOM4-130-3 (SEQ ID NO:218), DOM4-130-4 (SEQ ID NO:219), DOM4-130-5 (SEQ ID NO:220), DOM4-130-6 (SEQ ID NO:221), DOM4-130-7 (SEQ ID NO:222), DOM4-130-8 (SEQ ID NO:223), DOM4-130-9 (SEQ ID NO:224), DOM4-130-10 (SEQ ID NO:225), DOM4-130-11 (SEQ ID NO:226), DOM4-130-12 (SEQ ID NO:227), DOM4-130-13 (SEQ ID NO:228), DOM4-130-14 (SEQ ID NO:229), DOM4-130-15 (SEQ ID NO:230), DOM4-130-16 (SEQ ID NO:231), DOM4-130-17 (SEQ ID NO:232), DOM4-130-18 (SEQ ID NO:233), DOM4-130-19 (SEQ ID NO:234), DOM4-130-20 (SEQ ID NO:235), DOM4-130-21 (SEQ ID NO:236), DOM4-130-22 (SEQ ID NO:237), DOM4-130-23 (SEQ ID NO:238), DOM4-130-24 (SEQ ID NO:239), DOM4-130-25 (SEQ ID NO:240), DOM4-130-26 (SEQ ID NO:241), DOM4-130-27 (SEQ ID NO:242), DOM4-130-28 (SEQ ID NO:243), DOM4-130-31 (SEQ ID NO:244), DOM4-130-32 (SEQ ID NO:245), DOM4-130-33 (SEQ ID NO:246), DOM4-130-34 (SEQ ID NO:247), DOM4-130-35 (SEQ ID NO:248), DOM4-130-36 (SEQ ID NO:249), DOM4-130-37 (SEQ ID NO:250), DOM4-130-38 (SEQ ID NO:251), DOM4-130-39 (SEQ ID NO:252), DOM4-130-40 (SEQ ID NO:253), DOM4-130-41 (SEQ ID NO:254), DOM4-130-42 (SEQ ID NO:255), DOM4-130-43 (SEQ ID NO:256), DOM4-130-44 (SEQ ID NO:257), DOM4-130-45 (SEQ ID NO:258), DOM4-130-46 (SEQ ID NO:259), DOM4-130-47 (SEQ ID NO:260), DOM4-130-48 (SEQ ID NO:261), DOM4-130-49 (SEQ ID NO:262), DOM4-130-50 (SEQ ID NO:263), DOM4-130-51 (SEQ ID NO:264), DOM4-130-52 (SEQ ID NO:265), DOM4-130-53 (SEQ ID NO:266), DOM4-130-54 (SEQ ID NO:267), DOM4-130-55 (SEQ ID NO:268), DOM4-130-56 (SEQ ID NO:269), DOM4-130-57 (SEQ ID NO:270), DOM4-130-58 (SEQ ID NO:271), DOM4-130-59 (SEQ ID NO:272), DOM4-130-60 (SEQ ID NO:273), DOM4-130-61 (SEQ ID NO:274), DOM4-130-62 (SEQ ID NO:275), DOM4-130-63 (SEQ ID NO:276), DOM4-130-64 (SEQ ID NO:277), DOM4-130-65 (SEQ ID NO:278), DOM4-130-66 (SEQ ID NO:279), DOM4-130-67 (SEQ ID NO:280), DOM4-130-68 (SEQ ID NO:281), DOM4-130-69 (SEQ ID NO:282), DOM4-130-70 (SEQ ID NO:283), DOM4-130-71 (SEQ ID NO:284), DOM4-130-72 (SEQ ID NO:285), DOM4-130-73 (SEQ ID NO:286), DOM4-130-74 (SEQ ID NO:287), DOM4-130-75 (SEQ ID NO:288), DOM4-130-76 (SEQ ID NO:289), DOM4-130-77 (SEQ ID NO:290), DOM4-130-78 (SEQ ID NO:291), DOM4-130-79 (SEQ ID NO:292), DOM4-130-80 (SEQ ID NO:293), DOM4-130-81 (SEQ ID NO:294), DOM4-130-82 (SEQ ID NO:295), DOM4-130-83 (SEQ ID NO:296), DOM4-130-84 (SEQ ID NO:297), DOM4-130-85 (SEQ ID NO:298), DOM4-130-86 (SEQ ID NO:299), DOM4-130-87 (SEQ ID NO:300), DOM4-130-88 (SEQ ID NO:301), DOM4-130-89 (SEQ ID NO:302), DOM4-130-90 (SEQ ID NO:303), DOM4-130-91 (SEQ ID NO:304), DOM4-130-92 (SEQ ID NO:305), DOM4-130-93 (SEQ ID NO:306), DOM4-130-94 (SEQ ID NO:307), DOM4-130-95 (SEQ ID NO:308), DOM4-130-96 (SEQ ID NO:309), DOM4-130-97 (SEQ ID NO:310), DOM4-130-98 (SEQ ID NO:311), DOM4-130-99 (SEQ ID NO:312), DOM4-130-100 (SEQ ID NO:313), DOM4-130-101 (SEQ ID NO:314), DOM4-130-102 (SEQ ID NO:315), DOM4-130-103 (SEQ ID NO:316), DOM4-130-104 (SEQ ID NO:317), DOM4-130-105 (SEQ ID NO:318), DOM4-130-106 (SEQ ID NO:319), DOM4-130-107 (SEQ ID NO:320), DOM4-130-108 (SEQ ID NO:321), DOM4-130-109 (SEQ ID NO:322), DOM4-130-110 (SEQ ID NO:323), DOM4-130-111 (SEQ ID NO:324), DOM4-130-112 (SEQ ID NO:325), DOM4-130-113 (SEQ ID NO:326), DOM4-130-114 (SEQ ID NO:327), DOM4-130-115 (SEQ ID NO:328), DOM4-130-116 (SEQ ID NO:329), DOM4-130-117 (SEQ ID NO:330), DOM4-130-118 (SEQ ID NO:331), DOM4-130-119 (SEQ ID NO:332), DOM4-130-120 (SEQ ID NO:333), DOM4-130-121 (SEQ ID NO:334), DOM4-130-122 (SEQ ID NO:335), DOM4-130-123 (SEQ ID NO:336), DOM4-130-124 (SEQ ID NO:337), DOM4-130-125 (SEQ ID NO:338), DOM4-130-126 (SEQ ID NO:339), DOM4-130-127 (SEQ ID NO:340), DOM4-130-128 (SEQ ID NO:341), DOM4-130-129 (SEQ ID NO:342), DOM4-130-130 (SEQ ID NO:343), DOM4-130-131 (SEQ ID NO:344), DOM4-130-132 (SEQ ID NO:345), DOM4-130-133 (SEQ ID NO:346), DOM4-131 (SEQ ID NO:347), DOM4-132 (SEQ ID NO:348), and DOM4-133 (SEQ ID NO:349).

95. The method of claim 92, wherein said respiratory disease is selected from the group consisting of lung inflammation, chronic obstructive pulmonary disease, asthma, pneumonia, hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease, influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X, pulmonary hypertension, pulmonary nocardiosis, pulmonary tuberculosis, pulmonary veno-occlusive disease, rheumatoid lung disease, sarcoidosis, and Wegener's granulomatosis.
